SEQUENCE LISTING (57 sequences)

SEQ ID NO: 1 - protein, 113 aa, Synthetic construct
DIVMTQSPSSLAMSVGQKVTMSCKSSQSLLNSSNQKNYLAWYQQKPRQSPKLLVYFASTRDSGVPDRFLGSGSGTDFTLTINRVQAEDLADYFCQQHYILPPTFGAGTKLELK

SEQ ID NO: 2 - protein, 117 aa, Synthetic construct
EVQLQQSGPELVKPGASVKISCKTSGYTFTEYTMHWVKQSHGKSLEWIGSINPNNGGTNYNQKFKGKATLTVDKSSNTAYMELRSLTSGDSAVYYCARTLYYAMDYWGQGTSVTVSS

SEQ ID NO: 3 - protein, 109 aa, Synthetic construct
QAVVTQESALTTSPGETVTLTCRSSTGAVTTSNYANWVQEKPDHLFTGLIGGTNNRAPGVPARFSGSLIGDKAALTITGAQTEDEAIYHCALWYSNLWVFGGRTKLTVL

SEQ ID NO: 4 - protein, 119 aa, Synthetic construct
QVQLKESGPGLVAPSQSLSITCTVSGFSLTSYGVHWVRQPPGKGLEWLGVIWAGGVTHYNSALMSRLNISKDNSQSQVFLKMNSLRTDDTAMYYCARGGALLRSHFDYWGQGTTLTVSS

SEQ ID NO: 5 - protein, 107 aa, Synthetic construct
DIQMTQSPASLSASVGETVTITCRASENIFIYLAWYQQKQGKSPQLLVYNAKTLAEGVPSRFSGSGSGTQFSLKINSLLPEDFGSYYCQHHYGIPFTFGSGTKLEIE

SEQ ID NO: 6 - protein, 120 aa, Synthetic construct
EVQLQQSGAELVKPGASVKLSCTVSGFNITDTYIHWVKQRPEKGLEWIGRIDPANGNTKYDPKLQGKATITADTSSNTAYLQLSSLTSEDTAVYYCARDGTYVYYAMDYWGQGTSVTVSS

SEQ ID NO: 7 - protein, 107 aa, Synthetic construct
DILLTQSPAILSVSPGDRVSFSCRASQTIGTNIHWYHQTTNGSPRLLIKYASESISGIPSRFSGSGSGTDFTLTIISVESEDIADYYCQQSNSWPLTFGAGTKLELK

SEQ ID NO: 8 - protein, 121 aa, Synthetic construct
QVQLQQPGAELVRPGASVNLSCKASGYTFTNYWINWVKQRPGQGLEWIGNIHPYDSYTHYNQKFKGKATLTVDKSSSTAYMQLSSPTSEDSAVYFCTRRGVRRDDYYFDYWGQGTTLTVSS

SEQ ID NO: 9 - RNA, 20 nt, Synthetic construct
  source 1..20: mol_type = other RNA
aagtcctccgtctcctgcaa

SEQ ID NO: 10 - RNA, 20 nt, Synthetic construct
  source 1..20: mol_type = other RNA
cttccattgtctcattagct

SEQ ID NO: 11 - RNA, 20 nt, Synthetic construct
  source 1..20: mol_type = other RNA
acttccattgtctcattagc

SEQ ID NO: 12 - RNA, 20 nt, Synthetic construct
  source 1..20: mol_type = other RNA
ctctttgtgttgatgaatcc

SEQ ID NO: 13 - RNA, 20 nt, Synthetic construct
  source 1..20: mol_type = other RNA
tttgtgttgatgaatcctgg

SEQ ID NO: 14 - RNA, 20 nt, Synthetic construct
  source 1..20: mol_type = other RNA
gctcacaatcacaggccttg

SEQ ID NO: 15 - RNA, 20 nt, Synthetic construct
  source 1..20: mol_type = other RNA
ttcaggacattactattgcg

SEQ ID NO: 16 - RNA, 20 nt, Synthetic construct
  source 1..20: mol_type = other RNA
gcaagcaatatcctattacc

SEQ ID NO: 17 - RNA, 20 nt, Synthetic construct
  source 1..20: mol_type = other RNA
attgcgtggtaaggtaagag

SEQ ID NO: 18 - RNA, 20 nt, Synthetic construct
  source 1..20: mol_type = other RNA
ttggtttgttcctgctaagg

SEQ ID NO: 19 - RNA, 20 nt, Synthetic construct
  source 1..20: mol_type = other RNA
gcactttgtttggcctactg

SEQ ID NO: 20 - protein, 17 aa, Synthetic construct
KSSQSLLNSSNQKNYLA

SEQ ID NO: 21 - protein, 7 aa, Synthetic construct
FASTRDS

SEQ ID NO: 22 - protein, 9 aa, Synthetic construct
QQHYILPPT

SEQ ID NO: 23 - protein, 5 aa, Synthetic construct
EYTMH

SEQ ID NO: 24 - protein, 17 aa, Synthetic construct
SINPNNGGTNYNQKFKG

SEQ ID NO: 25 - protein, 8 aa, Synthetic construct
TLYYAMDY

SEQ ID NO: 26 - DNA, 339 bp, Synthetic construct
  source 1..339: mol_type = other DNA
gacattgtgatgacacagtctccatcctccctggctatgtcagtaggacagaaggtcactatgagctgcaagtccagtcagagccttttaaatagtagtaatcaaaagaactatttggcctggtaccagcagaaaccaagacagtctcctaaacttctggtatactttgcatccactagggattctggggtccctgatcgcttcttaggcagtggatctgggacagatttcactcttaccatcaaccgtgtgcaggctgaagacctggcagattacttctgtcagcaacattatattcttcctcccacgttcggtgctgggaccaagctggagctgaaa

SEQ ID NO: 27 - DNA, 351 bp, Synthetic construct
  source 1..351: mol_type = other DNA
gaggtccagctgcaacagtctggacctgagctggtgaagcctggggcttcagtgaagatatcctgcaagacttctggatacacattcactgaatacaccatgcactgggtgaagcagagccatggaaagagccttgagtggattggaagtattaatcctaacaatggtggtactaactacaaccagaagttcaagggcaaggccacattgactgtagacaagtcctccaacacagcctacatggagctccgcagcctgacatctggtgattctgcagtctattactgtgcaagaactctctactatgctatggactactggggtcaaggaacctcagtcaccgtctcctca

SEQ ID NO: 28 - protein, 14 aa, Synthetic construct
RSSTGAVTTSNYAN

SEQ ID NO: 29 - protein, 7 aa, Synthetic construct
GTNNRAP

SEQ ID NO: 30 - protein, 9 aa, Synthetic construct
ALWYSNLWV

SEQ ID NO: 31 - protein, 5 aa, Synthetic construct
SYGVH

SEQ ID NO: 32 - protein, 16 aa, Synthetic construct
VIWAGGVTHYNSALMS

SEQ ID NO: 33 - protein, 11 aa, Synthetic construct
GGALLRSHFDY

SEQ ID NO: 34 - DNA, 327 bp, Synthetic construct
  source 1..327: mol_type = other DNA
caggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatcactgtgctctatggtacagcaacctttgggtgttcggtggaagaaccaaactgactgtccta

SEQ ID NO: 35 - DNA, 357 bp, Synthetic construct
  source 1..357: mol_type = other DNA
caggtgcagctgaaggagtcaggacctggcctggtggcgccctcacagagcctgtccatcacttgcactgtctctgggttttcattaaccagctatggtgtacactgggttcgccagcctccaggaaagggtctggagtggctgggagtaatatgggctggtggagtcacacattataattcggctctcatgtccagactgaacatcagcaaagacaactcccagagccaagttttcttaaaaatgaacagtctacgaactgatgacacagccatgtactactgtgccagaggtggggcattacttcggtctcactttgactactggggccaaggcaccactctcacagtctcctca

SEQ ID NO: 36 - protein, 11 aa, Synthetic construct
RASENIFIYLA

SEQ ID NO: 37 - protein, 7 aa, Synthetic construct
NAKTLAE

SEQ ID NO: 38 - protein, 9 aa, Synthetic construct
QHHYGIPFT

SEQ ID NO: 39 - protein, 5 aa, Synthetic construct
DTYIH

SEQ ID NO: 40 - protein, 17 aa, Synthetic construct
RIDPANGNTKYDPKLQG

SEQ ID NO: 41 - protein, 11 aa, Synthetic construct
DGTYVYYAMDY

SEQ ID NO: 42 - DNA, 321 bp, Synthetic construct
  source 1..321: mol_type = other DNA
gacatccagatgactcagtctccagcctccctatctgcatctgtgggagaaactgtcaccatcacatgtcgagcaagtgagaatattttcatttatttagcatggtatcagcaaaaacagggaaaatctcctcagctcctggtctataatgcaaagaccttagcagaaggtgtgccatcaaggttcagtggcagtggatcaggcacacagttttctctgaagatcaacagccttctgcctgaagattttgggagttattactgtcaacatcattatggtattccattcacgttcggctcggggacaaagttggaaatagaa

SEQ ID NO: 43 - DNA, 360 bp, Synthetic construct
  source 1..360: mol_type = other DNA
gaggttcagctgcagcagtctggggcagagcttgtgaagccaggggcctcagtcaagttgtcctgcacagtttctggtttcaacattacagacacctatatacactgggtgaagcagaggcctgaaaagggcttggagtggattggaaggattgatcctgcgaatggtaatactaaatatgacccgaagttacagggcaaggccactataacagcagacacatcctccaacacagcctacctgcagctcagcagcctgacatctgaggacactgccgtctattactgtgctagagatggtacctacgtttactatgctatggactactggggtcaaggaacctcagtcaccgtctcctca

SEQ ID NO: 44 - protein, 11 aa, Synthetic construct
RASQTIGTNIH

SEQ ID NO: 45 - protein, 7 aa, Synthetic construct
YASESIS

SEQ ID NO: 46 - protein, 9 aa, Synthetic construct
QQSNSWPLT

SEQ ID NO: 47 - protein, 5 aa, Synthetic construct
NYWIN

SEQ ID NO: 48 - protein, 17 aa, Synthetic construct
NIHPYDSYTHYNQKFKG

SEQ ID NO: 49 - protein, 12 aa, Synthetic construct
RGVRRDDYYFDY

SEQ ID NO: 50 - DNA, 321 bp, Synthetic construct
  source 1..321: mol_type = other DNA
gacatcttgctgactcagtctccagccatcctgtctgtgagtccaggagacagagtcagtttctcctgcagggccagtcagaccattggcacaaacatacactggtatcaccaaacaacaaatggttctccaaggcttctcattaagtatgcttctgagtctatctctgggatcccttccaggtttagtggcagtggatcagggacagattttactcttaccatcatcagtgtggagtctgaagatattgcagattattactgtcaacaaagtaatagctggccgctcacgttcggtgctgggaccaagctggagctgaaa

SEQ ID NO: 51 - DNA, 363 bp, Synthetic construct
  source 1..363: mol_type = other DNA
caggtccaactgcagcagcctggggctgagctggtgaggcctggggcttcagtgaacctgtcctgcaaggcttctggctacaccttcaccaactactggataaactgggtgaagcagaggcctggacaaggccttgagtggatcggaaatattcatccttatgatagttatactcactacaatcaaaagttcaagggcaaggccacattgactgtagacaaatcctccagcacggcctacatgcagctcagcagcccgacatctgaggactctgcagtctatttctgtacaaggaggggggtacgacgggacgactactactttgactactggggccaaggcaccactctcacagtctcctca

SEQ ID NO: 52 - DNA, 1710 bp, Homo sapiens
  source 1..1710: mol_type = other DNA
atgaaagtgttactcagacttatttgtttcatagctctactgatttcttctctggaggctgataaatgcaaggaacgtgaagaaaaaataattttagtgtcatctgcaaatgaaattgatgttcgtccctgtcctcttaacccaaatgaacacaaaggcactataacttggtataaagatgacagcaagacacctgtatctacagaacaagcctccaggattcatcaacacaaagagaaactttggtttgttcctgctaaggtggaggattcaggacattactattgcgtggtaagaaattcatcttactgcctcagaattaaaataagtgcaaaatttgtggagaatgagcctaacttatgttataatgcacaagccatatttaagcagaaactacccgttgcaggagacggaggacttgtgtgcccttatatggagttttttaaaaatgaaaataatgagttacctaaattacagtggtataaggattgcaaacctctacttcttgacaatatacactttagtggagtcaaagataggctcatcgtgatgaatgtggctgaaaagcatagagggaactatacttgtcatgcatcctacacatacttgggcaagcaatatcctattacccgggtaatagaatttattactctagaggaaaacaaacccacaaggcctgtgattgtgagcccagctaatgagacaatggaagtagacttgggatcccagatacaattgatctgtaatgtcaccggccagttgagtgacattgcttactggaagtggaatgggtcagtaattgatgaagatgacccagtgctaggggaagactattacagtgtggaaaatcctgcaaacaaaagaaggagtaccctcatcacagtgcttaatatatcggaaattgaaagtagattttataaacatccatttacctgttttgccaagaatacacatggtatagatgcagcatatatccagttaatatatccagtcactaatttccagaagcacatgattggtatatgtgtcacgttgacagtcataattgtgtgttctgttttcatctataaaatcttcaagattgacattgtgctttggtacagggattcctgctatgattttctcccaataaaagcttcagatggaaagacctatgacgcatatatactgtatccaaagactgttggggaagggtctacctctgactgtgatatttttgtgtttaaagtcttgcctgaggtcttggaaaaacagtgtggatataagctgttcatttatggaagggatgactacgttggggaagacattgttgaggtcattaatgaaaacgtaaagaaaagcagaagactgattatcattttagtcagagaaacatcaggcttcagctggctgggtggttcatctgaagagcaaatagccatgtataatgctcttgttcaggatggaattaaagttgtcctgcttgagctggagaaaatccaagactatgagaaaatgccagaatcgattaaattcattaagcagaaacatggggctatccgctggtcaggggactttacacagggaccacagtctgcaaagacaaggttctggaagaatgtcaggtaccacatgccagtccagcgacggtcaccttcatctaaacaccagttactgtcaccagccactaaggagaaactgcaaagagaggctcacgtgcctctcgggtag

SEQ ID NO: 53 - DNA, 1731 bp, Mus musculus
  source 1..1731: mol_type = other DNA
atggagaatatgaaagtgctactggggctcatttgtctcatggtgcctctgctgtcgctggagattgacgtatgtacagaatatccaaatcagatcgttttgtttttatctgtaaatgaaattgatattcgcaagtgtcctcttactccaaataaaatgcacggcgacaccataatttggtacaagaatgacagcaagacccccatatcagcggaccgggactccaggattcatcagcagaatgaacatctttggtttgtacctgccaaggtggaggactcaggatattactattgtatagtaagaaactcaacttactgcctcaaaactaaagtaaccgtaactgtgttagagaatgaccctggcttgtgttacagcacacaggccaccttcccacagcggctccacattgccggggatggaagtcttgtgtgcccttatgtgagttattttaaagatgaaaataatgagttacccgaggtccagtggtataagaactgtaaacctctgcttcttgacaacgtgagcttcttcggagtaaaagataaactgttggtgaggaatgtggctgaagagcacagaggggactatatatgccgtatgtcctatacgttccgggggaagcaatatccggtcacacgagtaatacaatttatcacaatagatgaaaacaagagggacagacctgttatcctgagccctcggaatgagacgatcgaagctgacccaggatcaatgatacaactgatctgcaacgtcacgggccagttctcagaccttgtctactggaagtggaatggatcagaaattgaatggaatgatccatttctagctgaagactatcaatttgtggaacatccttcaaccaaaagaaaatacacactcattacaacacttaacatttcagaagttaaaagccagttttatcgctatccgtttatctgtgttgttaagaacacaaatatttttgagtcggcgcatgtgcagttaatatacccagtccctgacttcaagaattacctcatcgggggctttatcatcctcacggctacaattgtatgctgtgtgtgcatctataaagtcttcaaggttgacatagtgctttggtacagggactcctgctctggttttcttccttcaaaagcttcagatggaaagacatacgatgcctatattctttatcccaagaccctgggagaggggtccttctcagacttagatacttttgtttttaaactgttgcctgaggtcttggagggacagtttggatacaagctgttcatttatggaagggatgactatgttggagaagataccatcgaggttactaatgaaaatgtaaagaaaagcaggaggctgattatcattctagtgagagatatgggaggcttcagctggctgggccagtcatctgaagagcaaatagccatatacaatgctctcatccaggaaggaattaaaatcgtcctgcttgagttggagaaaatccaagactatgagaaaatgccagattctattcagttcattaagcagaaacacggagtcatttgctggtcaggagactttcaagaaagaccacagtctgcaaagaccaggttctggaaaaacttaagataccagatgccagcccaacggagatcaccattgtctaaacaccgcttactaaccctggatcctgtgcgggacactaaggagaaactgccggcagcaacacacttaccactcggctag

SEQ ID NO: 54 - protein, 573 aa, Synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIE

SEQ ID NO: 55 - DNA, 1719 bp, Synthetic construct
  source 1..1719: mol_type = other DNA
atggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgag

SEQ ID NO: 56 - protein, 833 aa, Synthetic construct
KPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 57 - DNA, 2499 bp, Synthetic construct
  source 1..2499: mol_type = other DNA
aagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

(19) United States
(12) Patent Application Publication
(10) Pub. No.: US 2025/0313608 A1
(43) Pub. Date: Oct. 9, 2025

(54) MATERIALS AND METHODS FOR TREATING CANCER

(71) Applicant: Mayo Foundation for Medical Education and Research, Rochester, MN (US)

(72) Inventors: Saad J. Kenderian, Rochester, MN (US); Kun Yun, Rochester, MN (US)

(21) Appl. No.: 19/173,238

(22) Filed: Apr. 8, 2025

Related U.S. Application Data
(60) Provisional application No. 63/631,710, filed on Apr. 9, 2024.

(51) Int. Cl. C07K 14/715; A61K 40/11; A61K 40/31; A61K 40/42; C07K 16/28

(52) U.S. Cl. CPC C07K 14/7155; A61K 40/11; A61K 40/31; A61K 40/4217; C07K 16/2866; A61K 2239/10; C07K 2317/76; C07K 2319/30

(57) ABSTRACT

This document provides methods and materials involved in treating cancer. For example, methods and materials for modulating (e.g., increasing or decreasing) an interleukin-1 (IL-1) signaling pathway (e.g., an IL-1βsignaling pathway) during an adoptive cell therapy (e.g., a chimeric antigen receptor (CAR) T cell therapy) are provided. In some cases, one or more inhibitors of an interleukin-1 receptor antagonist (IL-1RA) polypeptide can be used to increasing IL-1 signaling (e.g., to reduce immunosuppression of the administered cells). In some cases, CAR T cells having a reduced level of an interleukin 1 receptor, type I (IL-1R1) polypeptide can have decreased IL-1 signaling (e.g., to reduce T cell toxicity associated with the administered cells).

### CROSS-REFERENCE TO RELATED APPLICATIONS

This application claims the benefit of U.S. Patent Application Ser. No. 63/631,710, filed on Apr. 9, 2024. The disclosure of the prior application is considered part of, and is incorporated by reference in, the disclosure of this application.

### SEQUENCE LISTING

This application contains a Sequence Listing that has been submitted electronically as an XML file named “07039-2322001.xml.” The XML file, created on Mar. 30, 2025, is 63,096 bytes in size. The material in the XML file is hereby incorporated by reference in its entirety.

### TECHNICAL FIELD

This document relates to methods and materials involved in treating cancer. For example, this document provides methods and materials for modulating (e.g., increasing or decreasing) an interleukin-1 (IL-1) signaling pathway (e.g., an IL-1β signaling pathway) during an adoptive cell therapy (e.g., a chimeric antigen receptor (CAR) T cell therapy). In some cases, one or more inhibitors of an interleukin-1 receptor antagonist (IL-1RA) polypeptide can be used to increasing IL-1 signaling (e.g., to reduce immunosuppression of the administered cells). In some cases, CAR T cells having a reduced level of an interleukin 1 receptor, type I (IL-1R1) polypeptide can have decreased IL-1 signaling (e.g., to reduce T cell toxicity associated with the administered cells).

### BACKGROUND

Autologous CAR T cell therapy has shown remarkable clinical success in treating relapsed/refractory B-cell malignancies, but most patients relapse within 1-3 years following treatment (Yun et al., Leukemia., 37:1953-1962 (2023); Neelapu et al., New. Eng. Jour. Med., 377:2531-2544 (2017)). Numerous mechanisms of CAR T resistance have been studied including the immunosuppressive tumor microenvironment, yet these mechanisms are not fully understood (Shah et al., Nat. Rev. Clin. Onc., 16:372-385 (2019)). Thus, there is a need to overcome the resistance and improve the efficacy of CAR T cell therapy.

### SUMMARY

This document relates to methods and materials for treating cancer. As demonstrated herein, adoptive cell therapies (e.g., CAR T cell therapies) that are performed in the presence of IL-1 signaling are less susceptible to T cell immunosuppression (e.g., T cell inhibition mediated by the immunosuppressive tumor microenvironment (TME)). For example, this document provides methods and materials for increasing an IL-1 signaling pathway (e.g., an IL-1β signaling pathway) during an adoptive cell therapy (e.g., a CAR T cell therapy) to reduce immunosuppression of the administered cells (e.g., thereby improving T cell function and antitumor activity). In some cases, one or more inhibitors of an IL-1RA polypeptide can be used to reduce immunosuppression of the administered cells (e.g., thereby improving T cell function and antitumor activity) and/or to reduce T cell toxicity.

This document also provides methods and materials for making and/or using T cells (e.g., CAR T cells) having a reduced level of an IL-1R1 polypeptide. Also as demonstrated herein, adoptive cell therapies (e.g., CAR T cell therapies) associated with cytotoxicity had elevated IL-1 signaling. In some cases, T cells (e.g., CAR T cells) having a reduced level of an IL-1R1 polypeptide can be less likely to induce T cell toxicity. For example, a T cell (e.g., a CAR T cell) can be engineered to knock out (KO) a nucleic acid encoding an IL-1R1 polypeptide to reduce IL-1R1 polypeptide expression in that T cell. In some cases, T cells (e.g., CAR T cells) having a reduced level of an IL-1R1 polypeptide can have reduced cytotoxicity and can be administered (e.g., in an adoptive cell therapy) to a mammal (e.g., a human) having cancer to treat the mammal's cancer.

In general, one aspect of this document features methods for treating a mammal having cancer where the methods can include, or consist essentially of, administering, to the mammal, (a) an inhibitor of an IL-1RA polypeptide and (b) an adoptive cell therapy, where a number of cancer cells within the mammal is reduced. The mammal can be a human. The cancer can be a mantle cell lymphoma (MCL), a diffuse large B cell lymphoma (DLBCL), a Hodgkin's lymphoma, a non-Hodgkin lymphoma, an acute lymphoblastic leukemia (ALL), a chronic lymphocytic leukemia (CLL), an acute myeloid leukemia (AML), a germ cell tumor, a hepatocellular carcinoma, a bowel cancer, a lung cancer, a breast cancer, an ovarian cancer, a melanoma, a brain cancer, or a multiple myeloma. The inhibitor of the IL-1RA polypeptide can be an inhibitor of IL-1RA polypeptide expression. The inhibitor of the IL-1RA polypeptide can be an inhibitor of IL-1RA polypeptide activity. The inhibitor of the IL-1RA polypeptide can be an anti-IL-1RA antibody. The adoptive cell therapy is a CAR T cell therapy. The CAR can target a tumor-associated antigen. The inhibitor of the IL-1RA polypeptide and the adoptive cell therapy can be administered to the mammal simultaneously. The inhibitor of the IL-1RA polypeptide and the adoptive cell therapy can be administered to the mammal as a single composition. The inhibitor of the IL-1RA polypeptide and the adoptive cell therapy can be administered to the mammal separately. The inhibitor of the IL-1RA polypeptide can reduce immunosuppression of the adoptive cell therapy.

In another aspect, this document features T cells having a reduced likelihood of causing a CAR T cell-associated toxicity, where the T cell comprises (a) a reduced level of an IL-1R1 polypeptide, and (b) nucleic acid encoding a CAR, and where the T cell expresses the CAR. The T cell can have a disruption in at least one endogenous allele encoding the IL-1R1 polypeptide. The T cell can have a disruption in both endogenous alleles encoding the IL-1R1 polypeptide. The T cell can express a reduced level of the IL-1R1 polypeptide as compared to a comparable T cell lacking the disruption. The CAR can target a tumor-associated antigen. The T cell can be obtained from a human. The CAR T cell toxicity can be a cytokine release syndrome (CRS) or an immune effector cell-associated neurotoxicity syndrome (ICANS).

In another aspect, this document features methods for treating a mammal having cancer where methods can include, or consist essentially of, administering, to the mammal, a composition comprising a T cell having a reduced likelihood of causing a CAR T cell-associated toxicity, where the T cell comprises (a) a reduced level of an IL-1R1 polypeptide, and (b) nucleic acid encoding a CAR, and where the T cell expresses the CAR. The mammal can be a human. The cancer can be a MCL, a DLBCL, a Hodgkin's lymphoma, a non-Hodgkin lymphoma, an ALL, a CLL, an AML, a germ cell tumor, a hepatocellular carcinoma, a bowel cancer, a lung cancer, a breast cancer, an ovarian cancer, a melanoma, a brain cancer, or a multiple myeloma. The T cell can have a disruption in at least one endogenous allele encoding the IL-1R1 polypeptide. The T cell can have a disruption in both endogenous alleles encoding the IL-1R1 polypeptide. The T cell can express a reduced level of the IL-1R1 polypeptide as compared to a comparable T cell lacking the disruption. The CAR can target a tumor-associated antigen. The T cell can be obtained from a human.

In another aspect, this document features methods for providing a mammal with CAR T cells having a reduced likelihood of inducing a CAR T cell-associated toxicity. The methods can include, or consist essentially of, administering, to a mammal, a composition comprising a T cell having a reduced likelihood of causing a CAR T cell-associated toxicity, where the T cell comprises (a) a reduced level of an IL-1R1 polypeptide, and (b) nucleic acid encoding a CAR, and where the T cell expresses the CAR, where the CAR T cells do not induce the CAR T cell-associated toxicity as rapidly as comparable CAR T cells not having the reduced level of the IL-1R1 polypeptide administered to a comparable mammal. The mammal can be a human. The cancer can be a MCL, a DLBCL, a Hodgkin's lymphoma, a non-Hodgkin lymphoma, an ALL, a CLL, an AML, a germ cell tumor, a hepatocellular carcinoma, a bowel cancer, a lung cancer, a breast cancer, an ovarian cancer, a melanoma, a brain cancer, or a multiple myeloma. The T cell can have a disruption in at least one endogenous allele encoding the IL-1R1 polypeptide. The T cell can have a disruption in both endogenous alleles encoding the IL-1R1 polypeptide. The T cell can express a reduced level of the IL-1R1 polypeptide as compared to a comparable T cell lacking the disruption. The CAR can target a tumor-associated antigen. The CAR T cell toxicity can be a CRS or an ICANS.

In another aspect, this document features methods for providing a mammal with CAR T cells having a reduced susceptibility to T cell immunosuppression. The methods can include, or consist essentially of, (a) administering a composition comprising CAR T cells to a mammal, and (b) administering an inhibitor of an IL-1RA polypeptide to the mammal, where the CAR T cells do not exhibit T cell immunosuppression within the mammal as rapidly as comparable CAR T cells administered to a comparable mammal not administered the inhibitor of the IL-1RA polypeptide. The mammal can be a human. The CAR T cells can target a tumor-associated antigen. The inhibitor of the IL-1RA polypeptide can be an inhibitor of IL-1RA polypeptide expression. The inhibitor of the IL-1RA polypeptide can be an inhibitor of IL-1RA polypeptide activity. The inhibitor of the IL-1RA polypeptide can be an anti-IL-1RA antibody. The inhibitor of the IL-1RA polypeptide and the composition can be administered to the mammal simultaneously. The inhibitor of the IL-1RA polypeptide and the composition can be administered to the mammal as a single composition. The inhibitor of the IL-1RA polypeptide and the composition can be administered to the mammal separately.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIGS. 1A-1I. Ex vivo polarized M2-like macrophages inhibited antigen-specific proliferation of T cells containing a CAR targeting CD19 (CART19 cells) in a contact-independent manner. FIG. 1A) Schema of in vitro cocultures with freshly isolated monocytes or ex vivo polarized M2-like macrophages. CART19-282 cells were generated from healthy T cell donors. CD14+CD16-classical monocytes were isolated by negative selection magnetic beads. CART19 cells, isolated monocytes or differentiated M2-like macrophages, and JeKo-1 cells were cocultured at a ratio of 2:1:2 for 3 days. CART19 cell proliferation was measured by flow cytometry on day 3. Supernatants from cocultures were harvested and prepared for cytokine profiling by 38-multiplex assay. FIG. 1B) Schema of polarization of M2-like macrophages from classical monocytes. Isolated monocytes were cultured with recombinant human GM-CSF at 10 ng/mL for 7 days to generate M0 macrophage. M0 macrophages were further polarized into IL-4 differentiated M2-like macrophages by culturing with recombinant human IL-4 at 20 ng/ml or into M2-like macrophages by culturing with JeKo-1 cells at a ratio of 1:2 for another 24 hours. FIG. 1C) Confirmation of M2-like macrophage phenotypes in differentiated macrophages following the procedure described in FIG. 1B was performed by flow cytometry. FIG. 1D) CAR T cell antigen-specific proliferation was assessed in the classical monocyte cocultures. Four biological replicates were analyzed. Multiple paired t-test was used; ns, not significant. FIG. 1E) CAR T cell antigen-specific proliferation was assessed in the M2-like macrophage cocultures. Six biological replicates were analyzed. Multiple pair t-test was used; ** p<0.01. FIG. 1F) CAR T cell antigen-specific proliferation was tested using a TRANSWELL® assay. M2-like macrophages were seeded in the bottom chamber of a TRANSWELL® plate (0.4 μm), followed by plating of CART19 cells and JeKo-1 cells in the upper well at a ratio of 1:1. CART19 cell proliferation was measured on day 3. Three biological replicates were analyzed. Multiple paired t-test was used. * p<0.05. FIG. 1G) Heatmap of individually normalized concentrations of secreted molecules in cocultures with freshly isolated monocytes or M2-like macrophages. FIG. 1H) Cytokine secretion was compared between freshly isolated monocytes and ex vivo polarized M2-like macrophages incubated with untransduced (UTD) T cells. Paired t-test was used, ns, not significant, *p<0.05. UTD: control untransduced T cells. FIG. 1I) Cytokine secretion was compared between cocultures with or without ex vivo polarized M2-like macrophages with activated CART19 cells. Paired t-test was used, *p<0.05. FIGS. 1G-1I) 3 biological replicates were analyzed in total.

FIGS. 2A-2B. Downregulated IL-1β signaling and its downstream pathways were detected in baseline CAR T cell populations from non-responders. Overall, recovered cryopreserved brexucabtagene autoleucel (brexu-cel) products from 5 responders, 3 relapsed, and 4 non-responders were qualified for downstream single-cell RNA sequencing analysis. FIG. 2A) Identities of peripheral cell populations projected onto Uniform Manifold Approximation and Projection (UMAP) with each section indicating one cell type with resolution 0.5 (n=90,177 cells). FIG. 2B) Dot plots showing differentially regulated pathways of interest comparing non-responders and initial responders within CD4⁺ CART19 cell populations (n=19,711 cells), HSP-high CD8⁺ memory CART19 cell populations (n=18,333 cells), proliferative CD8⁺ CART19 cell populations (n=12,353), activated CD8⁺ CART19 cell populations (n=8,261 cells), and cytotoxic CD8⁺ CART19 cell populations (n=4,589). Downregulated pathways in non-responders are shown.

FIGS. 3A-3G. M2-like phenotypes and downregulation of IL-1β pathways were detected in peripheral monocyte populations from non-responders. Overall, recovered cryopreserved PBMCs from 3 responders, 3 relapsed, and 1 non-responder were qualified for downstream single-cell RNA sequencing analysis. FIG. 3A) Identities of peripheral cell populations projected onto UMAP with each section indicating one cell type with resolution 0.5 (n=41,002 cells). FIG. 3B) Volcano plots showing differentially expressed genes comparing non-responders and initial responders (relapsed and responders) within the M2-like monocyte (n=11,344 cells), M1-like monocyte (n=9,195 cells), and classical monocyte populations (n=1,849 cells) with the top 50 and the differentially expressed genes of interest labeled. FIG. 3C) Dot plots showing differentially regulated pathways of interest comparing non-responders and initial responders within the M2-like monocyte and M1-like monocyte populations. All pathways of interest presented above were downregulated in non-responders. FIG. 3D) Expression of genes in the IL-1 pathway and monocyte-related genes were identified by the differential gene expression analysis projected onto the UMAP. FIG. 3E-3G) Violin plots showing expression of cytokines with non-significant differential expression but involved in upstream or downstream of IL-1β production comparing the non-responders and the initial responders within each cell cluster of interest. Stars and P values indicated significance and upregulation of the labeled groups. * p<0.05, ** p<0.01, *** p<0.001, **** p<0.001.

FIGS. 4A-4D. Presence of M2-like macrophages promoted IL-1RA expression in both T cells and tumor cells. FIG. 4A) Expression of IL-1RA in monocytes and M2-like macrophages was plotted. JeKo-1 cells and UTD cells were cocultured with freshly isolated monocytes at a ratio of 2:2:1 overnight. The next day, 1X brefeldin A was added to the cocultures and the cells were incubated for another 24 hours before intracellular staining for IL-1RA in the UTD cells. In parallel, JeKo-1 cells and UTD cells were cocultured with ex vivo polarized M2-like macrophages at a ratio of 2:2:1 for 3 days. On day 3, 1X brefeldin A was added to the cocultures and the cells were incubated for another 24 hours before intracellular staining for IL-1RA in the UTD cells. FIG. 4B) Expression of IL-1RA in UTD cells after being cocultured with JeKo-1 cells and monocytes or ex vivo polarized M2 macrophages from the same coculture conditions from FIG. 4A. FIG. 4C) Expression of IL-1RA in JeKo-1 cells from the same coculture conditions from FIG. 4A. Three biological replicates were analyzed. FIG. 4D) JeKo-1 cells expressed intracellular IL-1RA. Multiple paired t-test was used. ns: not significant, ** p<0.01, *** p<0.001. UTD: control untransduced T cells.

FIGS. 5A-5D. Neutralization of IL-1RA mitigated CART19 cell proliferation inhibition by recombinant human IL-1RA and promoted CART19 cell anti-tumor activities in mantle cell lymphoma (MCL) xenografts in NOD-SCID-γ^{-/-} (NSG) mice. FIG. 5A) Recombinant human IL-1RA inhibited IL-1β dependent CART19 cell proliferation, which was restored by IL-1RA neutralizing antibody. CART19 cells were cocultured with JeKo-1 cells at a ratio of 1:1 for 3 days with addition of 100 μg/mL recombinant human IL-1β, 100 pg/mL IL-1β+100 ng/ml IL-1RA, or 100 pg/mL IL-1β+100 ng/ml IL-1RA+20 μg/mL human IL-1RA neutralizing antibody (clone 10309). PBS was used as a vehicle control. Three biological replicates were analyzed in total. Multiple t-test was used. FIG. 5B) Immunofluorescence staining (IF) confirmed the M2-like phenotype of engrafted macrophages and production of IL-1RA 7 days after tumor injections. 5×10⁶ ex vivo polarized M0 macrophages and 10×10⁶ luciferase⁺ JeKo-1 cells were resuspended in MATRIGEL® and subcutaneously engrafted in NSG mice. Tumor tissues were harvest, cryopreserved, and cross-sectioned for IF staining. Images, presented at 40X, were analyzed on ImageJ. Luciferase⁺ JeKo-1 cells expressed GFP as well. Human CD206 and IL-1RA were stained with rabbit-anti-human monoclonal antibody followed by goat-anti-rabbit secondary antibody Cyanine5. The same procedures were conducted on tumors composed of 10×10⁶ luciferase⁺ JeKo-1 cells. FIG. 5C) Schematic outline of the in vivo experimental design. NSG mice were engrafted with ex vivo polarized M0 macrophages (Mφ) and luciferase⁺ JeKo-1 cells were delivered with MATRIGEL® as indicated. Mice engrafted with only luciferase⁺ JeKo-1 cells were used as a control. After tumor establishment, mice were treated with CART19 cells intravenously in combination with 10 mg/kg IL-1RA neutralizing antibody (clone 10309) or control IgG (C1.18.4, BioXCell) by intraperitoneal injections twice a week for 3 weeks. Tumor burden was measured periodically by bioluminescence imaging. FIG. 5D) Tumor burden was monitored over time. One-way ANOVA was used to compare CART19⁺ control IgG containing M2 Mφ with CART19⁺ IL-1RA neutralizing antibody (neuAb) containing M2 Mφ or CART19⁺ control IgG at each timepoint. * p<0.05, ** p<0.01, **** p<0.0001.

FIGS. 6A-6D. IL-1RA inhibition of IL-1β mediated CART19 cells proliferation was dependent on interactions with IL-1R1 on stimulated CART19 cells. FIG. 6A) IL-1R1 expression was tested in both CD4⁺ and CD8⁺ CART19 cells under indicated stimulation conditions. CART19 cells were cocultured with JeKo-1 cells at a ratio of 1:1 for 3 days. Expression of IL-1R1 was measured in both CD4⁺ and CD8⁺ T cells by flow cytometry. Three biological replicates were analyzed in total. Two-way ANOVA was used. ** p<0.01, ns: not significant. FIG. 6B) IL-1R1 expression was measured in CD4⁺ CAR T cells after being cocultured with ex vivo polarized M2-like macrophages. CART19 cells, JeKo-1 cells and ex vivo polarized M2-like macrophages were cocultured for 3 days as described in FIG. 1A. Expression of IL-1R1 was measured in CD4⁺ CART19 cells daily by flow cytometry. Three biological replicates were analyzed in total. Two-way ANOVA was used. *** p<0.001, **** p<0.0001, ns: not significant. FIG. 6C) Confirmation of reduced IL-1R1 polypeptides in CART19 cells treated with a single guide RNA (sgRNA) designed to target and knock out (KO) expression of IL-1R1 (IL-1R1 KO CART19 cells). IL-1R1 KO CART19 cells and CART19 cells treated with a control sgRNA (CTsgRNA CART19 cells) were cocultured with JeKo-1 cells at a ratio of 1:1 for 2 days. Expression of IL-1R1 on CD4⁺ T cells was measured by flow cytometry. Multiple t-test was used. ** p<0.01, ns, not significant. Three biological replicates were analyzed in total. FIG. 6D) Functional confirmation of IL-1R1 KO CART19 cells according to response to IL-1β stimulation. IL-1R1 KO CART19 cells were cocultured with JeKo-1 cells at a ratio of 1:1 with supplement of vehicle control, 100 μg/mL recombinant human (rh) IL-1β), or 100 pg/mL rhIL-1β+100 ng/mL rhIL-1RA for 3 days. CART19 cell antigen specific proliferation was measured on day 3 by flow cytometry. Three biological replicates were analyzed in total. Multiple t-test was used.

FIGS. 7A-7D. FIG. 7A) Heatmap of normalized percentages of CART19 cells that were positive for inhibitory receptors TRAIL-R2, Fas, PD-1, and PD-L1 after 3 days of coculture with freshly isolated monocytes or M2-like macrophages in the presence or absence of JeKo-1 cells. FIG. 7B) Heatmap of normalized expression level (MFI) of inhibitory receptors TRAIL-R2, Fas, PD-1, and PD-L1 in CART19 cells after 3 days of coculture with freshly isolated monocytes or M2-like macrophages in the presence or absence of JeKo-1 cells. FIG. 7C) Heatmap of normalized percentages of monocytes or M2-like macrophages that were positive for inhibitory ligands TRAIL, FasL, PD-L1, and PD-L2 after 3 days of coculture with CART19 cells in the presence of JeKo-1 cells. FIG. 7D) Heatmap of normalized expression level (MFI) of inhibitory ligands TRAIL, FasL, PD-L1, and PD-L2 in monocytes or M2-like macrophages after 3 days of coculture with CART19 cells in the presence of JeKo-1 cells. Two biological replicates were analyzed.

FIG. 8. Effects of rhIL-1β and IL-1RA on IL-1R1 KO CART19 cell antigen-specific proliferation were evaluated. Cell viability of CTsgRNA and IL-1R1 KO CAR T cells was tested on day 8 of CART19 cell production. Five biological replicates were analyzed in total. Paired t-test was used. ns, not significant.

FIG. 9. CART cell proliferation indicated by CFSE intensity changes. Briefly, CFSE-stained CART cells were cocultured with JeKo-1 and monocytes or M2-like macrophages for 3 days. CFSE intensity was measured prior to coculture and on day 3 by flow cytometry.

FIGS. 10A-10C. NSG mice were engrafted with ex vivo polarized M0 macrophages (Mφ) and luciferase⁺ JeKo-1 cells delivered with matrigel as shown in FIG. 5C. After tumor establishment, mice were treated with CART19 intravenously in combination with IL-1ra neutralizing antibody or control IgG (C1.18.4, BioXCell) or UTD in combination with IL-1ra neutralizing antibody or control IgG. Tumor burden was measured by bioluminescence imaging twice a week until survival endpoint was reached. FIGS. 10A and 10B) Tumor burden changes over time. Two-way ANOVA was used. * p<0.05. ** p<0.01, *** p<0.0001. FIG. 10C) Kaplan-Meier survival curve of the treatment groups. Log-rank (Mantel-Cox) test was used. * p<0.05. UTD, untransduced T cell. Median overall survival of each treatment group is listed as follow: JeKo-1+Mφ_CART19+IL-1ra mAb: not reached, JeKo-1+Mφ_CART19+IgG control: 52 days, JeKo-1_CART19+IgG control: not reached, JeKo-1+Mφ_UTD+IL-1ra mAb: 40 days, JeKo-1+Mφ_UTD+IgG control: 43 days, JeKo-1_UTD+IL-1ra mAb: 43 days, JeKo-1_UTD+IgG control: 43 days.

FIG. 11. Downregulated IL-1β response and its downstream pathways were detected in baseline CART subset populations from non-responders in the phase 2 ZUMA-2 trial. Overall, recovered cryopreserved brexu-cel products from 5 responders, 3 relapsed, and 4 non-responders were qualified for downstream single-cell RNA sequencing analysis. Bar graph showing proportion of different cell types in baseline brexu-cel products comparing non-responders (n=4) and initial responders (n=8).

FIGS. 12A-12E. IL-1β promotes in vitro CART cell proliferation and activation as well as enhances CART antitumor activities in vivo. FIG. 12A) CART cell cycle state analysis by EdU assay. EdU reagent was added to CART-JeKo-1 coculture prior to a 24-hour incubation. EdU was detected in IL-1RI⁺ and IL-1RI⁻ T cell subsets at 24 hours via flow cytometry. 3 biological replicates were analyzed in total. Multiple paired t-test was used. *p<0.05. FIG. 12B) CART cell division indicated by CFSE intensity changes with supplementation of IL-1β. Briefly, CFSE-stained CART cells were cocultured with JeKo-1 for 3 days supplemented with different doses of IL-1β. CFSE intensity was measured in CART prior to coculture and on day 3 by flow cytometry. FIG. 12C) CART apoptosis assay indicated by Annexin V/7-AAD. CART cells were cocultured with JeKo-1 for 3 days supplemented with different doses of IL-1β. Expression of 7-AAD and Annexin V was measured in T cells by flow cytometry. 3 biological replicates were analyzed in total. Two-way ANOVA was used to compare bulk CD3⁺ vs IL-1RI⁺ T cells under the indicated conditions. * p<0.05, ** p<0.01. FIG. 12D) Schema of recombinant human IL-1β supplementation during CART cell production prior to intravenous injection in NSG mice. FIG. 12E) Tumor burden changes of IL-1ß-pre-exposed CART in an MCL xenograft mouse model. NSG mice intravenously engrafted with 1×10⁶ luciferase⁺ JeKo-1 were treated with 1×10⁶ CART cells with or without IL-1β pre-exposure once tumors were established based on BLI. Tumor burden was monitored over time by BLI. Two-way ANOVA was used to compare CART with IL-1β-pre-exposed CART treated group. * p<0.05.

FIG. 13. M2-like phenotype induced by different multiple myeloma cell lines. Classical monocytes were differentiated into M0 macrophages followed by further incubation with 20 ng/ml IL-4, OPM-2, or RPMI-8226 cells as shown in FIG. 1B. Expression of CD206 and CD163 was measured by flow cytometry at the end of coculture.

FIG. 14. IL-4- and JeKo-1-differentiated M2-like macrophages show similar immunosuppression capacity on CART19 antigen-specific expansion. CART cells were cocultured with JeKo-1 and IL-4 (20 ng/ml)- or JeKo-1-differentiated M2-like macrophages at a ratio of 2:2:1 for 3 days. CART antigen-specific expansion was measured on day 3 by flow cytometry. 3 biological replicates were analyzed in total. Multiple paired t-test was used. * p<0.5.

FIG. 15. CART cell apoptosis indicated by Annexin V and 7-AAD. CART cells were cocultured with JeKo-1 and fresh monocytes or M2-like macrophages for 3 days. Expression of 7-AAD and Annexin V was measured on day 3 by flow cytometry. 3 biological replicates were analyzed in total. Multiple paired t-test was used. ns, not significant.

FIGS. 16A-16B. A 7-day coculture of JeKo-1, CART19, and fresh monocytes or M2-like macrophages showing significantly suppressed CART proliferation by M2-like macrophages. FIG. 16A) CART antigen-specific proliferation after 7 days of coculture with JeKo-1 and monocytes or M2-like macrophages. CART, JeKo-1, and monocytes or M2-like macrophages were cocultured at a ratio of 2:2:1. CART antigen-specific expansion was measured on day 7 by flow cytometry. 3 biological replicates were analyzed in total. Multiple paired t-test was used. *** p<0.001, ns, not significant. 3 biological replicates were analyzed in total. FIG. 16B) Cell division of CART cells indicated by CFSE intensity. CSFE-stained CART cells were cocultured with JeKo-1 and monocytes or M2-like macrophages at a ratio of 2:2:1 for 7 days. CFSE intensity was measured prior to cocultures and on day 7.

FIG. 17. Fold-change of CART antigen-specific expansion in M2-like macrophage cocultures. Fold-change of CART antigen-specific expansion was calculated by the ratio of the absolute number of CD3 T cells in JeKo-1+M2+ CART19 coculture to the absolute number of CD3 T cells in JeKo-1+CART19 coculture shown in FIGS. 1E and 1F.

FIG. 18. Dose titration of rh IL-1β, IL-1ra, and IL-1ra neutralizing antibodies. CART cells were cocultured with JeKo-1 at a ratio of 1:1 for 3 days with supplementation of different doses of IL-1β, IL-1ra, and IL-1ra neutralizing antibody as indicated. 3 biological replicates were analyzed in total. One-way ANOVA was used. * p<0.05, ** p<0.01, *p<0.001.

FIG. 19. Inhibition of CART antigen-specific expansion by M2-like macrophage-conditioned media. CART cells were cocultured with JeKo-1 at a ratio of 1:1 in fresh cell culture media, M2-like macrophage-conditioned media, or M2-like macrophage-conditioned media supplemented with 100 μg/mL IL-1β for 3 days, 3 biological replicates were analyzed in total. One-way ANOVA was used. *** p<0.001, **** p<0.0001.

FIG. 20. M2- and M1-like phenotype assessment in macrophages engrafted in NSG mice. Confirmation of M2-like phenotype of engrafted macrophages 7 days after tumor injections by immunofluorescence (IF) staining. Ex vivo polarized M0 macrophages and luciferase+ JeKo-1 were subcutaneously engrafted in NSG mice. Tumor tissues were harvested and prepared for IF staining. Luciferase+ JeKo-1 generated in the lab expressed GFP as well (green); human CD206 or iNOS was stained with mouse-anti-human antibody conjugated with AF547 (red) or rabbit-anti-human antibody conjugated with AF647 (magenta), respectively; DAPI was presented in blue.

FIG. 21. Kaplan-Meier progression-free survival curve of the treatment groups. Log-rank (Mantel-Cox) test was used. * p<0.05. UTD, untransduced T cell. Median PFS survival of each treatment group is listed as follow: JeKo-1+Mφ_CART19+IL-1ra mAb: not reached, JeKo-1+Mφ_CART19⁺ IgG control: 35 days, JeKo-1_CART19+IgG control: not reached, JeKo-1+Mφ_UTD+IL-1ra mAb: 21 days, JeKo-1+Mφ_UTD+IgG control: 21 days, JeKo-1_UTD+IL-1ra mAb: 24 days, JeKo-1_UTD+IgG control: 28 days.

FIG. 22. Bar graph showing proportion of different cell types in the baseline PBMCs comparing non-responder to initial responders.

FIGS. 23A-23B. Expression of identified differentially expressed genes in patient circulating monocyte populations. FIGS. 23A and 23B) Violin plots showing expression of HMOX1, EGR1, IL1RN, CPNE1, and TRIM33 in the baseline monocyte populations (M2-like monocyte (n=11,344 cells), M1-like monocyte (n=9,195 cells), classical monocyte (n=1,849 cells)) of the analyzed patients (N=7) and blood monocytes (n=1,228 cells) or classical monocytes (n=10,591 cells) from healthy individuals (N=9) extracted from publicly available scRNAseq datasets. **** p<0.0001.

FIG. 24. Expression of inhibitory markers on IL-1RI+ and IL-1RI− T cells. CART cells were cocultured with JeKo-1 at a ratio of 1:1 for 24 hours. Expression of LAG3, CTLA4, and PD-1 was measured on both IL-1RI+ and IL-1RI− T cells by flow cytometry. 3 biological replicates were analyzed in total. Multiple paired t-test was used. ** p<0.01, *** p<0.001.

FIG. 25. Intracellular expression of effector cytokines in CART cells stimulated with JeKo-1 for 4 hours supplemented with IL-1β. CART cells were cocultured with JeKo-1 at a ratio of 1:1 for 4 hours supplemented with different doses of IL-1β. Expression of granzyme B, IL-2, and IFN-γ were measured intracellularly in CART cells by flow cytometry. Multiple pair t-test was used. 3 biological replicates were analyzed in total.

FIG. 26. Tumor cytotoxicity of CART cells with addition of IL-1β. CART cells were cocultured with luciferase+ JeKo-1 at a ratio of 1:1 for 48 hours. Percentage of tumor killing was indicated by BLI. 3 biological replicates were analyzed in total. Multiple paired t-test was used.

### DETAILED DESCRIPTION

This document relates to methods and materials for treating cancer. For example, this document provides methods and materials for modulating (e.g., increasing or decreasing) an IL-1 signaling pathway (e.g., an IL-1β signaling pathway) during an adoptive cell therapy (e.g., a CAR T cell therapy) to improve the efficacy of the adoptive cell therapy. In some cases, one or more inhibitors of an IL-1RA polypeptide can be incorporated into adoptive T cell therapies (e.g., CAR T cell therapies) to reduce immunosuppression of the administered cells (e.g., thereby improving T cell function and antitumor activity). In some cases, CAR T cells having a reduced level of an IL-1R1 polypeptide can be used in an adoptive cell therapy (e.g., a CAR T cell therapy) as they are less likely to induce T cell toxicity (e.g., CAR T cell-associated toxicity).

Any appropriate method can be used to modulate (e.g., increase or decrease) an IL-1 signaling pathway (e.g., an IL-1β signaling pathway) during an adoptive cell therapy (e.g., a CAR T cell therapy). Methods for modulating (e.g., increasing or decreasing) an IL-1 signaling pathway can target any appropriate polypeptide within the IL-1 signaling pathway. Examples of polypeptides in the IL-1 signaling pathway that can be targeted include, without limitation, IL-1RA polypeptides and IL-1R1 polypeptides.

In some cases, one or more inhibitors of an IL-1RA polypeptide can be used to increase an IL-1 signaling pathway (e.g., an IL-1β signaling pathway). For example, one or more inhibitors of an IL-1RA polypeptide can be used to increase a level of an IL-1β polypeptide within a cell (e.g., as compared to a level of an IL-1β polypeptide in a comparable cell that is not administered one or more inhibitors of an IL-1RA polypeptide). In some cases, one or more inhibitors of an IL-1RA polypeptide can be administered before, together with, and/or after (e.g., before, together with, and after administration of) an adoptive cell therapy to increase an IL-1 signaling pathway (e.g., to reduce immunosuppression of the administered cells thereby improving T cell function and antitumor activity).

An inhibitor of an IL-1RA polypeptide can be any appropriate inhibitor of an IL-1RA polypeptide. An inhibitor of an IL-1RA polypeptide can be an inhibitor of IL-1RA polypeptide expression or IL-1RA polypeptide activity. Examples of compounds that can inhibit IL-1RA polypeptide activity include, without limitation, antibodies (e.g., neutralizing antibodies) that target (e.g., target and bind) to a IL-1RA polypeptide, and small molecules that target (e.g., target and bind) to a IL-1RA polypeptide. Examples of compounds that can inhibit of IL-1RA polypeptide expression include, without limitation, nucleic acid molecules designed to induce RNA interference of polypeptide expression of a IL-1RA polypeptide (e.g., a siRNA molecule or a shRNA molecule), antisense molecules, and miRNAs.

Examples of inhibitors of an IL-1RA polypeptide that can be used as described herein (e.g., to increase an IL-1 signaling pathway (e.g., an IL-1β signaling pathway) during an adoptive cell therapy to reduce immunosuppression of the T cells thereby improving T cell function and antitumor activity) include those set forth in Example 3. For example, an inhibitor of an IL-1RA polypeptide can be an antibody (e.g., a neutralizing antibody) that includes a light chain variable (VL) domain including the CDR sequences of Clone #1 (e.g., a VL domain set forth in SEQ ID NO:1) and/or a heavy chain variable (VH) domain including the CDR sequences of Clone #1 (e.g., a VH domain set forth in SEQ ID NO:2). For example, an inhibitor of an IL-1RA polypeptide can be an antibody (e.g., a neutralizing antibody) that includes a VL domain including the CDR sequences of Clone #2 (e.g., a VL domain set forth in SEQ ID NO:3) and/or a VH domain including the CDR sequences of Clone #2 (e.g., a VH domain set forth in SEQ ID NO:4). In another example, an inhibitor of an IL-1RA polypeptide can be an antibody (e.g., a neutralizing antibody) that includes a VL domain including the CDR sequences of Clone #3 (e.g., a VL domain set forth in SEQ ID NO: 5) and/or a VH domain including the CDR sequences of Clone #3 (e.g., a VH domain set forth in SEQ ID NO:6). In yet another example, an inhibitor of an IL-1RA polypeptide can be an antibody (e.g., a neutralizing antibody) that includes a VL domain including the CDR sequences of Clone #4 (e.g., a VL domain set forth in SEQ ID NO:7) and/or a VH domain including the CDR sequences of Clone #4 (e.g., a VH domain set forth in SEQ ID NO:8).

In some cases, an inhibitor of an IL-1RA polypeptide that can be used as described herein can be as described elsewhere (see, e.g., Fang et al., N. Engl. J. Med., 387:1524-1527 (2022) and Jarrell et al., J. Allergy Clin. Immunol., 149 (1): 358-368 (2022)).

In some cases, a reduced level of an IL-1R1 polypeptide can be used to decrease an IL-1 signaling pathway (e.g., an IL-1β signaling pathway). For example, T cells (e.g., CAR T cells) having (e.g., engineered to have) a reduced level of an IL-1R1 polypeptide can be used to treat cancer as described herein. For example, T cells having (e.g., engineered to have) a reduced level of an IL-1R1 polypeptide can have decreased IL-1 signaling pathway and can be used in an adoptive cell therapy (e.g., to reduce T cell toxicity associated with the administered cells).

In some cases, a T cell (e.g., a CAR T cell) having (e.g., engineered to have) a reduced level of an IL-1R1 polypeptide can be less likely to induce T cell toxicity (e.g., as compared to a T cell that not engineered to have a reduced level of an IL-1R1 polypeptide as described herein). For example, a mammal (e.g., a human such as a human having cancer) that is administered an adoptive cell therapy that includes one or more T cells (e.g., CAR T) cells having (e.g., engineered to have) a reduced level of an IL-1R1 polypeptide can be less likely to experience one or more CAR T cell-associated toxicities (e.g., cytokine release syndrome (CRS) and/or immune effector cell-associated neurotoxicity syndrome (ICANS)) in response to the adoptive cell therapy. Any appropriate method can be used to assess toxicity of T cells (e.g., T cells having a reduced level of an IL-1R1 polypeptide). Examples of methods that can be used to evaluate T cell (e.g., CAR T cell) cytotoxicity include, without limitation, cytotoxicity assays (e.g., to evaluate whether or not T cells (e.g., CAR T cells) are effective at killing target cells), effector cytokine quantification assays, and T cell phenotyping.

A T cell having (e.g., engineered to have) a reduced level of a polypeptide can be generated using any appropriate method. In some cases, a T cell (e.g., a CAR T cell) can be engineered to KO nucleic acid encoding an IL-1R1 polypeptide to reduce IL-1R1 polypeptide expression in that T cell (e.g., as compared to a comparable T cell that is not engineered to KO nucleic acid encoding an IL-1R1 polypeptide). For example, at least one endogenous allele (e.g., one allele or both alleles) of a nucleic acid encoding an IL-1R1 polypeptide can be disrupted (e.g., knocked out) to generate a T cell (e.g., a CAR T cell) having a reduced level of an IL-1R1 polypeptide. In another example, both endogenous alleles of a nucleic acid encoding an IL-1R1 polypeptide can be disrupted (e.g., knocked out) to generate a T cell (e.g., a CAR T cell) having a reduced level of an IL-1R1 polypeptide. A T cell that is engineered to KO nucleic acid encoding an IL-1R1 polypeptide can also be referred to herein as an IL-1R1 KO T cell, an IL-1R1^{-/-} T cell, an IL-1R1^{k/o} T cell, or an IL-1R1^{KO} T cell.

A T cell having (e.g., engineered to have) a reduced level of an IL-1R1 polypeptide can be any appropriate T cell. A T cell can be a naïve T cell. Examples of T cells that can be engineered to have a reduced level of an IL-1R1 polypeptide as described herein include, without limitation, cytotoxic T cells (e.g., CD4⁺ CTLs and/or CD8⁺ CTLs). For example, a T cell that can be engineered to have a reduced level of an IL-1R1 polypeptide can be a CAR T cell. In some cases, one or more T cells designed to have a reduced level of an IL-1R1 polypeptide can be T cells that were obtained from a mammal (e.g., a mammal having cancer) that is to be treated with those T cells designed to have a reduced level of an IL-1R1 polypeptide. For example, T cells can be obtained from a mammal that is to be treated as described herein.

The term “reduced level” as used herein with respect to a level of an IL-1R1 polypeptide refers to any level of that IL-1R1 polypeptide that is lower than a reference level of that IL-1R1 polypeptide in control T cells or any level of that IL-1R1 polypeptide that is lower in the post-engineered/treated T cells as compared to the level of that IL-1R1 polypeptide in the pre-engineered/treated version of those T cells. The term “reference level” as used herein with respect to an IL-1R1 polypeptide refers to the level of that polypeptide typically observed in control T cells from one or more healthy mammals (e.g., humans) not engineered to have a reduced level of that IL-1R1 polypeptide as described herein. Control T cells can include, without limitation, T cells that are wild-type T cells obtained from a healthy mammal. In some cases, a reduced level of an IL-1R1 polypeptide can be an undetectable level of that IL-1R1 polypeptide. In some cases, a reduced level of an IL-1R1 polypeptide can be an eliminated level of that IL-1R1 polypeptide.

When a T cell (e.g., a CAR T cell) is engineered to KO nucleic acid encoding an IL-1R1 polypeptide to reduce IL-1R1 polypeptide expression in that T cell, any appropriate method can be used to KO nucleic acid. Examples of techniques that can be used to knock out a nucleic acid encoding an IL-1R1 polypeptide include, without limitation, gene editing, homologous recombination, non-homologous end joining, and microhomology-mediated end joining. For example, gene editing (e.g., with engineered nucleases) can be used to knock out a nucleic acid encoding an IL-1R1 polypeptide, e.g., such that a full length polypeptide is no longer expressed. Examples of nucleases that can be used for genome editing include, without limitation, CRISPR-associated (Cas) nucleases, zinc finger nucleases (ZFNs), transcription activator-like effector (TALE) nucleases, homing endonucleases (HE; also referred to as meganucleases), prime editing, and base editing.

In some cases, a clustered regularly interspaced short palindromic repeat (CRISPR)/Cas system can be used (e.g., can be introduced into one or more T cells) to KO nucleic acid encoding an IL-1R1 polypeptide. A CRISPR/Cas system used to KO nucleic acid can include a guide RNA (gRNA) that is complementary to the target nucleic acid (e.g., nucleic acid encoding an IL-1R1 polypeptide). Examples of gRNAs that are specific to nucleic acid encoding an IL-1R1 polypeptide include, without limitation, gRNAs having the nucleic acid sequence AAGUCCUCCGUCUCCUGCAA (SEQ ID NO:9), CUUCCAUUGUCUCAUUAGCU (SEQ ID NO:10), ACUUCCAUUGUCUCAUUAGC (SEQ ID NO:11), CUCUUUGUGUUGAUGAAUCC (SEQ ID NO:12), UUUGUGUUGAUGAAUCCUGG (SEQ ID NO:13), GCUCACAAUCACAGGCCUUG (SEQ ID NO:14), UUCAGGACAUUACUAUUGCG (SEQ ID NO:15), GCAAGCAAUAUCCUAUUACC (SEQ ID NO:16), AUUGCGUGGUAAGGUAAGAG (SEQ ID NO:17), and UUGGUUUGUUCCUGCUAAGG (SEQ ID NO:18). In some cases, a gRNA can be designed based on a sequence of nucleic acid encoding an IL-1R1 polypeptide. Exemplary nucleic acids encoding an IL-1R1 polypeptide sequence include those set forth in Example 4.

A CRISPR/Cas system used to KO nucleic acid encoding an IL-1R1 polypeptide can include any appropriate Cas nuclease. Examples of Cas nucleases include, without limitation, Cas1, Cas2, Cas3, Cas9, Cas10, Cpf1, Cas12, and Cas13. In some cases, a Cas component of a CRISPR/Cas system designed to KO nucleic acid encoding an IL-1R1 polypeptide can be a Cas9 nuclease. For example, the Cas9 nuclease of a CRISPR/Cas9 system described herein can be a as set forth in Example 5. In some cases, a Cas component of a CRISPR/Cas system designed to KO nucleic acid encoding an IL-1R1 polypeptide can be as described elsewhere (see, e.g., Sterner et al., J. Vis. Exp., 22: (149) (2019)).

Components of a CRISPR/Cas system (e.g., a gRNA and a Cas nuclease) used to KO nucleic acid encoding an IL-1R1 polypeptide can be introduced into one or more T cells (e.g., CAR T cells) in any appropriate format. In some cases, a component of a CRISPR/Cas system can be introduced into one or more T cells as a nucleic acid encoding a gRNA and/or a nucleic acid encoding a Cas nuclease. For example, a nucleic acid encoding at least one gRNA and a nucleic acid encoding at least one Cas nuclease (e.g., a Cas9 nuclease) can be introduced into one or more T cells. In some cases, a component of a CRISPR/Cas system can be introduced into one or more T cells as a gRNA and/or as a Cas nuclease. For example, at least one gRNA and at least one Cas nuclease (e.g., a Cas9 nuclease) can be introduced into one or more T cells.

In some cases, a ZFN system can be used (e.g., can be introduced into one or more T cells) to KO nucleic acid encoding an IL-1R1 polypeptide. A ZFN system used to KO nucleic acid can include a polypeptide including (a) a DNA-binding domain (e.g., zinc fingers) that is complementary to a target nucleic acid (e.g., nucleic acid encoding an IL-1R1 polypeptide), and (b) a nuclease domain (e.g., a nuclease domain that can created double-strand breaks). A ZFN system used to KO nucleic acid encoding an IL-1R1 polypeptide can include any appropriate nuclease domain. In some cases, a nuclease domain of a ZFN system designed to KO nucleic acid encoding an IL-1R1 polypeptide can be a Fok1 nuclease domain.

In some cases, a TALEN system can be used (e.g., can be introduced into one or more T cells) to KO nucleic acid encoding an IL-1R1 polypeptide. A TALEN system used to KO nucleic acid can include a polypeptide including (a) a transcription activator-like (TAL) effector DNA-binding domain directing a nuclease to a target nucleic acid (e.g., nucleic acid encoding an IL-1R1 polypeptide), and (b) a nuclease domain (e.g., a nuclease domain that can created double-strand breaks). A TALEN system used to KO nucleic acid encoding an IL-1R1 polypeptide can include any appropriate nuclease. In some cases, a nuclease can be a non-specific nuclease. In some cases, a nuclease can function as a dimer. In some cases, a nuclease of a TALEN system designed to KO nucleic acid encoding an IL-1R1 polypeptide can be a Fok1 nuclease.

Components of a gene-editing system (e.g., a CRISPR/Cas system) used to KO nucleic acid encoding an IL-1R1 polypeptide can be introduced into one or more T cells (e.g., CAR T cells) using any appropriate method. A method of introducing components of a gene-editing system into a T cell can be a physical method. A method of introducing components of a gene-editing system into a T cell can be a chemical method. A method of introducing components of a gene-editing system into a T cell can be a particle-based method. Examples of methods that can be used to introduce components of a gene-editing system into one or more T cells include, without limitation, electroporation, transfection (e.g., lipofection), transduction (e.g., viral vector mediated transduction), microinjection, and nucleofection.

A T cell (e.g., a CAR T cell) having (e.g., engineered to have) a reduced level of an IL-1R1 polypeptide can express (e.g., can be engineered to express) any appropriate antigen receptor. In some cases, an antigen receptor can be a heterologous antigen receptor. In some cases, an antigen receptor can be a CAR. In some cases, an antigen receptor can be a tumor antigen (e.g., tumor-specific antigen) receptor. For example, a T cell can be engineered to express a tumor-specific antigen receptor that targets a tumor-specific antigen (e.g., a cell surface tumor-specific antigen) expressed by a cancer cell in a mammal having cancer. Examples of antigens that can be recognized by an antigen receptor expressed by a T cell having a reduced level of an IL-1R1 polypeptide as described herein include, without limitation, cluster of differentiation 19 (CD19), mucin 1 (MUC-1), human epidermal growth factor receptor 2 (HER-2), estrogen receptor (ER), epidermal growth factor receptor (EGFR), alphafetoprotein (AFP), carcinoembryonic antigen (CEA), CA-125, epithelial tumor antigen (ETA), melanoma-associated antigen (MAGE), CD33, CD123, CLL-1, E-Cadherin, folate receptor alpha, folate receptor beta, IL13R, EGFRviii, CD22, CD20, kappa light chain, lambda light chain, desmopressin, CD44v, CD45, CD30, CD5, CD7, CD2, CD38, BCMA, CD138, FAP, CS-1, and C-met. For example, a T cell having a reduced level of an IL-1R1 polypeptide can be designed to express an antigen receptor targeting CD19.

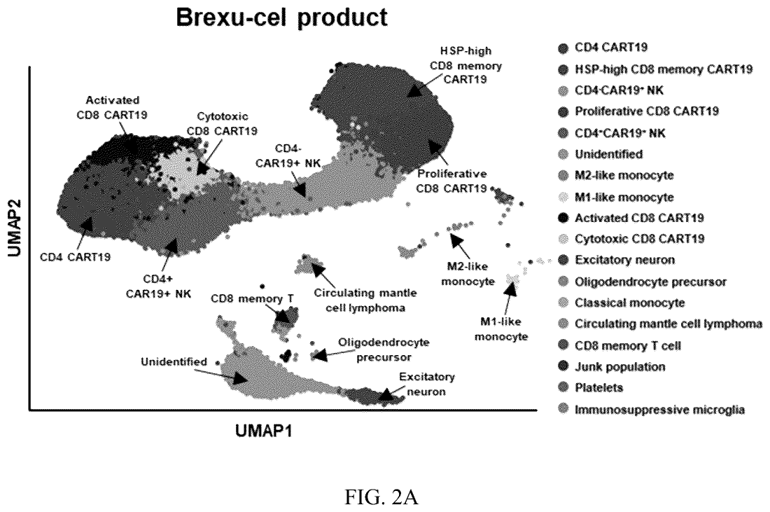

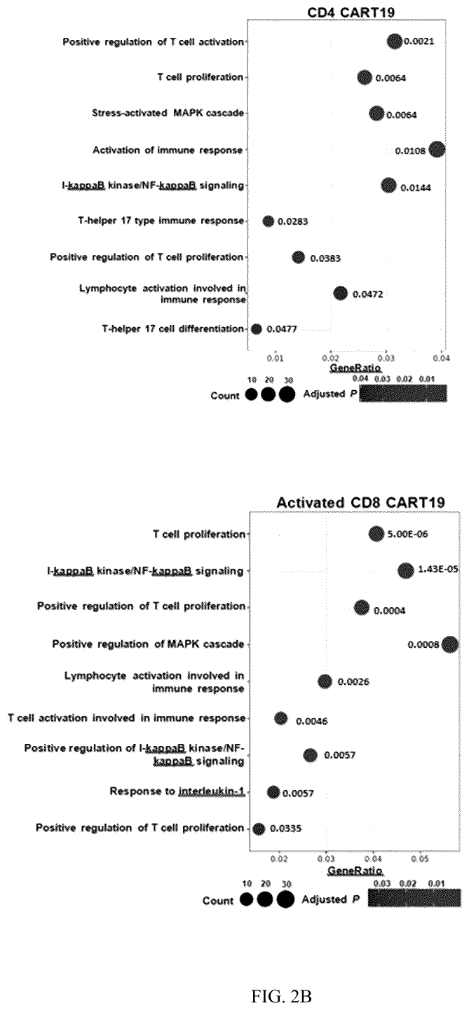

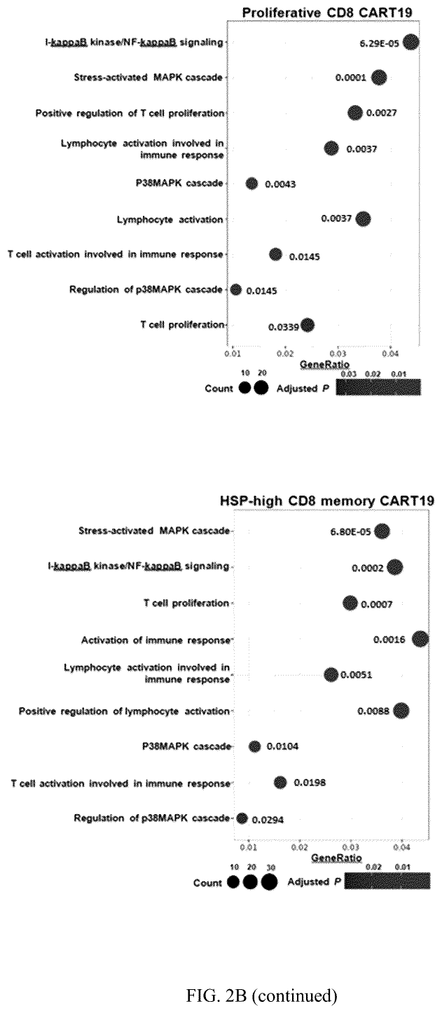

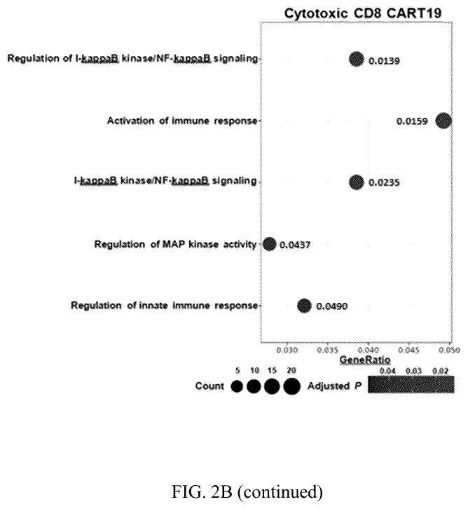

When an antigen receptor is a CAR, the CAR can be any appropriate CAR. A CAR can include an antigen-binding domain, an optional hinge, a transmembrane domain, and one or more signaling domains. Examples of antigen-binding domains include, without limitation, an antigen-binding fragment (Fab), a variable region of an antibody heavy (VH) chain, a variable region of a light (VL) chain, a single chain variable fragment (scFv), and domains from growth factors that bind to a cancer cell receptor (e.g., domains from EGF, PDGR, FGF, TGF, or derivatives thereof). In some cases, an antigen-binding domain of a CAR can target (e.g., can target and bind to) a cancer antigen or a cancer-specific antigen. In some cases, an antigen-binding domain of a CAR can be as described elsewhere (see, e.g., U.S. Patent Application Publication No. 2017/0183418 such as U.S. Patent Application Publication No. 2017/0183418 at paragraph and the sequence listing; U.S. Patent Application Publication No. 2017/0183413 such as U.S. Patent Application Publication No. 2017/0183413 at paragraph [0049], FIG. 2, Table 9, and the sequence listing; U.S. Patent Application Publication No. 2018/0291079 such as U.S. Patent Application Publication No. 2018/0291079 at paragraphs [0041]-[0045], and Table 4; U.S. Patent Application Publication No. 2020/0289563 such as U.S. Patent Application Publication No. 2020/0289563 at paragraphs [0006]-[0053], [0186]-[0189], and Table 1; and U.S. Patent Application Publication No. 2003/0211097 such as U.S. Patent Application Publication No. 2003/0211097 at paragraphs [0081] and [0211-0215] and the sequence listing.

In some cases, a CAR can include an optional hinge region. In some cases, a hinge region can be located between an antigen-binding domain and a transmembrane domain of a CAR. In some cases, a hinge region can provide a CAR with increased flexibility for the antigen-binding domain. For example, a hinge region can reduce spatial limitations of an antigen-binding domain of a CAR and its target antigen (e.g., to increase binding between an antigen-binding domain of a CAR and its target antigen). Examples of hinge regions that can be used as described herein include, without limitation, a membrane-proximal region from an IgG, a membrane-proximal region from CD8, and a membrane-proximal region from CD28. In some cases, a hinge region of a CAR can be as described elsewhere (see, e.g., U.S. Patent Application Publication No. 2018/0000914 such as U.S. Patent Application Publication No. 2018/0000914 at paragraph [0168], and Table 1; U.S. Patent Application Publication No. 2017/0183418 such as U.S. Patent Application Publication No. 2017/0183418 at paragraphs [0034], [0037], [0040], and Table 2; U.S. Patent Application Publication No. 2017/0183413 such as U.S. Patent Application Publication No. 2017/0183413 at paragraph [0116]; and U.S. Patent Application Publication No. 2017/0145094 such as U.S. Patent Application Publication No. 2017/0145094 at paragraph [0104].

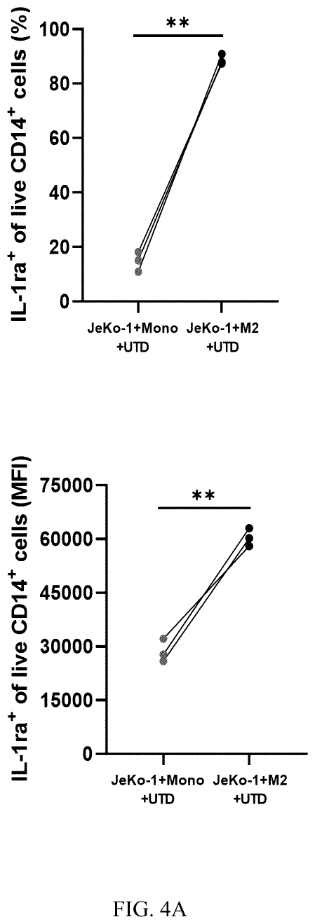

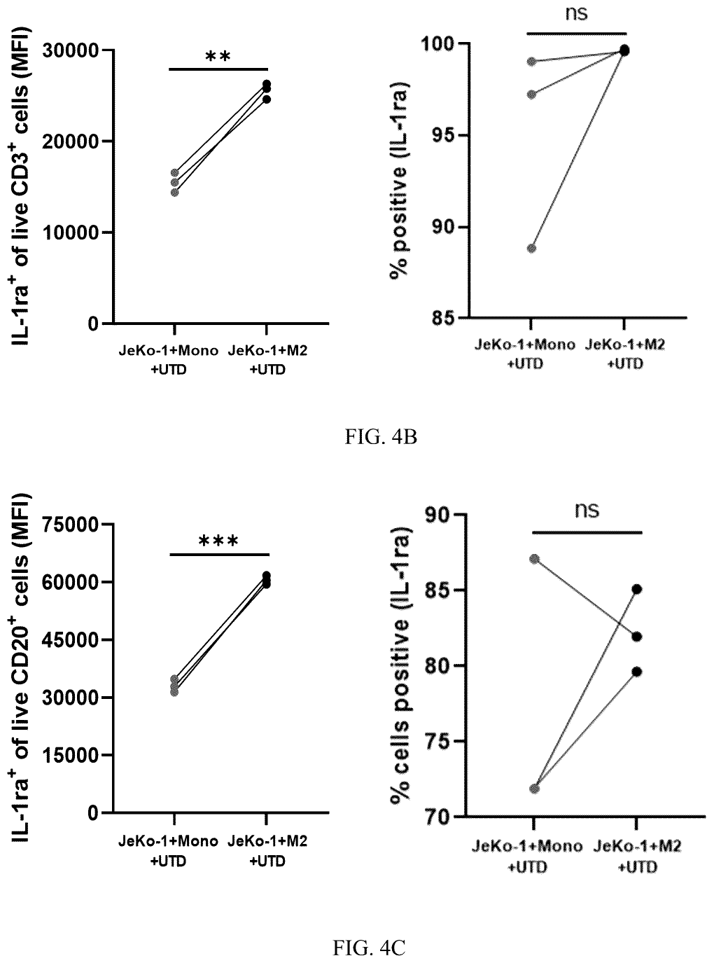

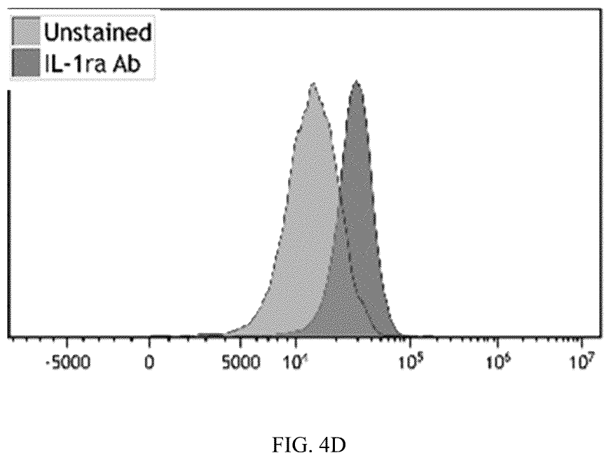

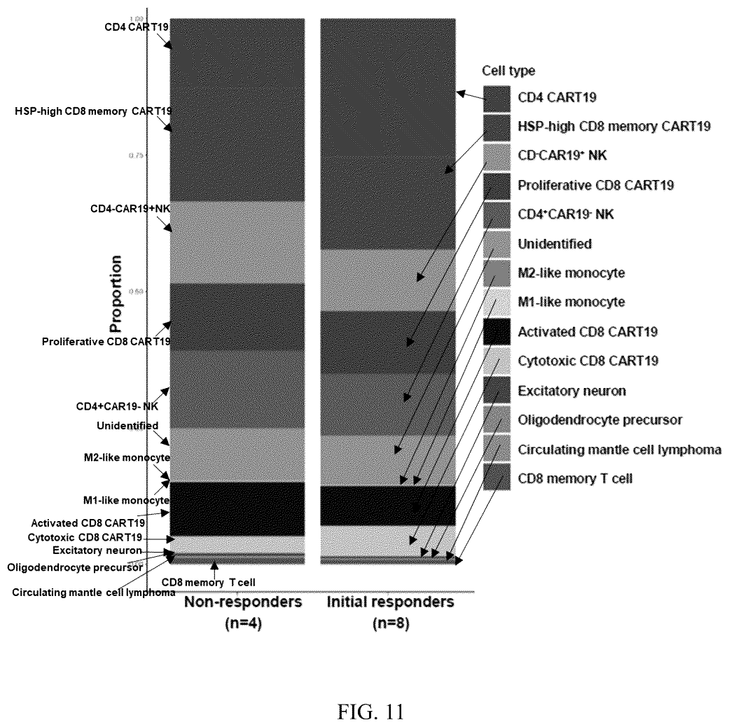

A CAR described herein can include any appropriate transmembrane domain. A transmembrane domain can be located between an antigen-binding domain and a signaling domain of a CAR and/or located between a hinge and a signaling domain of a CAR. In some cases, a transmembrane domain can provide structural stability for the CAR. For example, a transmembrane domain can include a structure (e.g., a hydrophobic alpha helix structure) that can span a cell membrane and can anchor the CAR to the plasma membrane. Examples of transmembrane domains that can be used as described herein include, without limitation, CD3ζ transmembrane domains, CD4 transmembrane domains, CD8 (e.g., a CD8α) transmembrane domains, CD28 transmembrane domains, CD16 transmembrane domains, and erythropoietin receptor transmembrane domains. In some cases, a transmembrane domain of a CAR can be as described elsewhere (see, e.g., U.S. Patent Application Publication No. 2016/0120906 such as U.S. Patent Application Publication No. 2016/0120906 at paragraphs [0155], [0161], [0269], FIG. 4, and FIG. 11; U.S. Patent Application Publication No. 2019/0209616 such as U.S. Patent Application Publication No. 2019/0209616 at paragraph [0026]; U.S. Patent Application Publication No. 2018/0000914 such as U.S. Patent Application Publication No. 2018/0000914 at paragraphs [0168]-[0171]; U.S. Patent Application Publication No. 2017/0183418 such as U.S. Patent Application Publication No. 2017/0183418 at paragraphs [0116]-[0118]; U.S. Patent Application Publication No. 2017/0183413 such as U.S. Patent Application Publication No. 2017/0183413 at paragraphs [0116]-[0118]; and U.S. Patent Application Publication No. 2017/0145094 such as U.S. Patent Application Publication No. 2017/0145094 at paragraphs [0104]-[0107].

A CAR described herein can include any appropriate signaling domain or combination of signaling domains (e.g., a combination of two, three, or four signaling domains). In some cases, a signaling domain of a CAR can be an intracellular signaling domain normally found within T cells or NK cells. Examples of signaling domains that can be used as described herein include, without limitation, BBC signaling domains, 28ζ signaling domains, CD2 signaling domains, CD3ζ signaling domains, CD28 signaling domains, Toll-like receptor (TLR) signaling domains (e.g., TLR3 or TLR4 signaling domains), CD27 intracellular signaling domains, OX40 (CD134) intracellular signaling domains, 4-1BB (CD137) intracellular signaling domains, CD278 intracellular signaling domains, DAP10 intracellular signaling domains, DAP12 intracellular signaling domains, FceRly intracellular signaling domains, CD278 intracellular signaling domains, CD122 intracellular signaling domains, CD132 intracellular signaling domains, CD70 intracellular signaling domains, cytokine receptor intracellular signaling domains, and CD40 intracellular signaling domains. In some cases, a CAR for use as described herein can be designed to be a first-generation CAR having a CD3ζ intracellular signaling domain. In some cases, a CAR for use as described herein can be designed to be a second-generation CAR having a CD28 intracellular signaling domain followed by a CD3ζ intracellular signaling domain. In some cases, a CAR for use as described herein can be designed to be a third generation CAR having (a) a CD28 intracellular signaling domain followed by (b) a CD27 intracellular signaling domain, an OX40 intracellular signaling domains, or a 4-1BB intracellular signaling domain followed by 5 (c) a CD3ζ intracellular signaling domain. In some cases, the intracellular signaling domain(s) of a CAR can be as described elsewhere (see, e.g., U.S. Patent Application Publication No. 2018/0000914 such as U.S. Patent Application Publication No. 2018/0000914 at paragraphs [0164]-[0167]; and U.S. Patent Application Publication No. 2017/0183413 such as U.S. Patent Application Publication No. 2017/0183413 at paragraphs [0112]-[0115].

In some cases, a CAR can be as set forth in Table 2.

TABLE 2
Exemplary CARs for targeting tumor antigens.

| Antigen | scFv antibody name | Reference(s) |
|---|---|---|
| CD19 | FMC63 | Milone et al., Mol. Ther., 17(8): 1453-64 (2009) (PMID: 26330164) |
| | MOR208 | Kellner et al., Leukemia, 27(7): 1595-8 (2013) (PMID: 23277329) |
| | Humanized scFv | International Patent Application Publication No. WO2015157252 |
| | 4G7 | EP Patent No. EP2997141 |
| | Low affinity scFv | Chinese Patent No. CN107406517 |
| | FMC63 CAR | Kang et al., Int. J. Mol. Sci., 21(23): 9163 (2020) |
| | KHYG-1 and 4G7 | |
| | CAR KHYG-1 | |
| MUC-1 | 5E5 | Posey et al., Immunity, 45(5): 947-948 (2016) (PMID: 27851918) |
| HER-2 | 4D5 | Ohnishi et al., Br. J. Cancer, 71(5): 969-73 (1995) (PMID: 7734322) |
| | | Forsberg et al., Cancer Res., 79(5): 899-904 (2019) (PMID: |
| | | 30622115) |
| | | Nellan et al., J. Immunother. Cancer, 6(1): 30 (2018) (PMID: |
| | | 29712574) |
| | | Priceman et al., Clin Cancer Res., 24(1): 95-105 (2018) (PMID: |
| | | 29061641) |
| | FRP5 | Bielamowics et al., Neuro. Oncol., 20(4): 506-518 (2018) (PMID: |
| | | 29016929) |
| | K70H-28z, K70L- | Zhou et al., Front. Cell Develop. Biol., 10: (2022). |
| | 28z, KSAbH-28z and | |
| | KSAbL-28z | |
| EGFR | M27 | Jiang et al., Cancer Immunol. Res., 6(11): 1314-1326 (2018) (PMID: |
| | | 30201736) |
| | Cetuximab | Caruso et al., Cancer Res., 75(17): 3505-18 (2015) (PMID: 26330164) |
| Folate receptor alpha | C4 based | Ao et al., J. Immunother., 42(8): 284-296 (2019) (PMID: 31261167) |
| | MOv19 | Song et al., J. Hematol. Oncol., 9(1): 56 (2016) (PMID: 27439908) |
| Mesothelin | SS1 | Haas et al., Mol. Ther., S1525-0016(19)30328-4 (2019) (PMID: |
| | | 31420241) |
| | M clone | Adusumilli et al., Sci. Transl. Med., 6(261): 261RA151 (2014) |
| | | (PMID: 25378643) |
| AFP | ET1402L1 | Liu et al., Clin. Cancer Res., 23(2): 478-488 (2017) (PMID: |
| | | 27535982) |
| CEA | Anti-CEA scFv | Chi et al., Cancer Med., 8(10): 4753-4765 (2019) (PMID: 31237116) |
| | CEACAM5 | Thistlethwaite et al., Cancer Immunol. Immunother., 66(11): 1425- |
| | | 1436 (2017) (PMID: 28660319) |
| | hMN14 | Katz et al., Clin. Cancer Res., 21(14): 3149-59 (2015) (PMID: |
| | | 25850950) |
| CD123 | 22172, 22176 | Gill et al., Blood, 123(15): 2343-54 (2014) (PMID: 24596416) |
| | Humanized scFv | US Patent Application Publication No. 2016/0068601 |
| | Humanized scFv | EP Patent No. EP2968415 |
| | Tagrazofusp based | Pemmaraju et al. N. Engl. J. Med., 380(17): 1628-1637 (2019) |
| | | (PMID: 31018069) |
| CD33 | MY96 | Kenderian et al., Leukemia, 29(8): 1637-47 (2015) (PMID: 25721896) |
| | Humanized scFv | International Patent Application Publication No. WO2016014576 |
| CLEC12A | Humanized scFv | US Patent Application Publication No. US20160051651 |
| | CLL1 scFv | International Patent Application Publication No. WO2016120219 |
| CD22 | M0971 | Fry et al., Nat. Med., 24(1): 20-28 (2018) (PMID: 29155426) |
| | Humanized scFv | International Patent Application Publication No. WO/2016/164731 |
| | clones | |
| | Inotuzumab based | Kantarjian et al., N. Engl. J. Med., 375(8): 740-53 (2016) (PMID: |
| | | 27292104) |
| | Moxetumomab based | Kreitman et al., Leukemia, 32(8): 1768-1777 (2018) (PMID: |
| | | 30030507) |
| CD20 | Rituximab | Zhang et al., Signal Transduct. Target Ther., 1: 16002 (2016) (PMID: |
| | | 29263894) |
| | Leu 16 | Lee et al., J. Immunother., 41(1): 19-31 (2018) (PMID: 29176334) |
| | CD20 scFvs | International Patent Application Publication No. WO/2016/164731 |
| BCMA | BCMA-02 | Raje et al., N. Engl. J. Med., 380(18): 1726-1737 (2019) (PMID: |
| | | 31042825) |
| | LCAR38 | Zhao et al., J. Hematol. Oncol., 11(1): 141 (2018) (PMID: 30572922) |
| | BCMA scFv | Smith et al., Cancer Immunol. Res., 7(7): 1047-1053 (2019) (PMID: |
| | | 31113804) |
| | Biepitopic | Xu et al., Proc. Natl. Acad. Sci. USA, 116(19): 9543-9551 (2019) |
| | | (PMID: 30988175) |
| | NVS BCMA | Cohen et al., J. Clin. Invest., 129(6): 2210-2221 (2019) (PMID: |
| | | 30896447) |
| CS-1 | CS1R | Wang et al., Clin. Cancer Res., 24(1): 106-119 (2018) (PMID: |
| | | 29061640) |
| | CS1 ScFv | Chu et al., Leukemia, 28(4): 917-27 (2014) (PMID: 24067492) |
| | Elotuzumab based | Dimopoulos et al., N. Engl. J. Med., 379(19): 1811-1822 (2018) |
| | | (PMID: 30403938) |
| CD138 | ScFv | Sun et al., Oncotarget., 10(24): 2369-2383 (2019) (PMID: 31040928) |
| CD44v6 | Humanized scFv | Leuci et al., Oncoimmunology, 7(5): e1423167 (2018) (PMID: |
| | | 29721373) |
| | cMAb U36 | Sandstrom et al., Int. J. Oncol., 40(5): 1525-32 (2012) (PMID: |
| | | 22307465) |
| NKG2D | scFv | Yang et al., J. Immunother. Cancer, 7(1): 171 (2019) (PMID: |
| | | 31288857) |
| | NKG2Dg scFv | Parihar et al., Cancer Immunol. Res., 7(3): 363-375 (2019) (PMID: |
| | | 30651290) |
| CD38 | Nanobody CD38 | An et al., Mol. Pharm., 15(10): 4577-4588 (2018) (PMID: 30185037) |
| | Humanized scFv | Yoshida et al., Clin. Transl. Immunology, 5(12): e116 (2016) (PMID: |
| | | 28090317) |
| GPRC5D | Humanized scFv | Smith et al., Sci. Transl. Med., 11(485)eaau7746 (2019) (PMID: |
| | | 30918115) |
| CD79b | scFv (L-H) and (H-L) | Ormhoj et al., Clin. Cancer Res., (2019) (PMID: 31439577) |
| CD103 | M290 | Zhang et al., Am. J. Transplant., 9(9): 2012-23 (2009) (PMID: |
| | | 19645708) |
| FAP | FAP5 | Wang et al., Cancer Immunol. Res., 2(2): 154-66 (2014) (PMID: |
| | | 24778279) |
| CD70 | Human CD70 | Park et al., Oral Oncol., 78: 145-150 (2018) (PMID: 29496042) |
| MUC16 | 4H11 | Koneru et al., Oncoimmunol., 4(3): e994446 (2015) (PMID: |
| | | 25949921) |
| IL13Ra2 | IL13R | Kong et al., Clin. Cancer Res., 18(21): 5949-60 (2012) (PMID: |
| | | 22966020) |

Any appropriate method can be used to express an antigen receptor on a T cell having (e.g., engineered to have) a reduced level of an IL-1R1 polypeptide. For example, nucleic acid encoding an antigen receptor can be introduced into one or more T cells. In some cases, viral transduction can be used to introduce nucleic acid encoding an antigen receptor into a non-dividing a cell. Nucleic acid encoding an antigen receptor can be introduced in a T cell using any appropriate method. In some cases, nucleic acid encoding an antigen receptor can be introduced into a T cell by transduction (e.g., viral transduction using a retroviral vector such as a lentiviral vector) or transfection. In some cases, nucleic acid encoding an antigen receptor can be introduced ex vivo into one or more T cells. For example, ex vivo engineering of T cells expressing an antigen receptor can include transducing isolated T cells with a lentiviral vector encoding an antigen receptor. In cases where T cells are engineered ex vivo to express an antigen receptor, the T cells can be obtained from any appropriate source (e.g., a mammal such as the mammal to be treated or a donor mammal, or a cell line).

In some cases, when a T cell having (e.g., engineered to have) a reduced level of an IL-1R1 polypeptide also expresses (e.g., is engineered to express) an antigen receptor, that T cell can be engineered to have a reduced level of an IL-1R1 polypeptide and engineered to express an antigen receptor using any appropriate method. In some cases, a T cell can be engineered to have a reduced level of an IL-1R1 polypeptide first and engineered to express an antigen receptor second, or vice versa. In some cases, a T cell can be simultaneously engineered to have a reduced level of an IL-1R1 polypeptide and to express an antigen receptor. For example, (a) one or more nucleic acids used to reduce a level of an IL-1R1 polypeptide, and (b) one or more nucleic acids encoding an antigen receptor (e.g., a CAR) can be simultaneously introduced into one or more T cells. One or more nucleic acids used to reduce a level of an IL-1R1 polypeptide, and one or more nucleic acids encoding an antigen receptor can be introduced into one or more T cells on separate nucleic acid constructs or on a single nucleic acid construct. One or more nucleic acids used to reduce a level of an IL-1R1 polypeptide, and one or more nucleic acids encoding an antigen receptor can be introduced ex vivo into one or more T cells. In cases where T cells are engineered ex vivo (a) to have a reduced level of an IL-1R1 polypeptide, and (b) to express an antigen receptor, the T cells can be obtained from any appropriate source (e.g., a mammal such as the mammal to be treated or a donor mammal, or a cell line).

In some cases, a T cell having (e.g., engineered to have) a reduced level of an IL-1R1 polypeptide can be stimulated. A T cell can be stimulated at the same time as being engineered to have a reduced level of an IL-1R1 polypeptide. For example, one or more T cells having a reduced level of an IL-1R1 polypeptide used in an adoptive cell therapy can be stimulated first, and can be engineered to have a reduced level of an IL-1R1 polypeptide second, or vice versa. A T cell can be stimulated using any appropriate method. For example, a T cell can be stimulated by contacting the T cell with one or more polypeptides. Examples of polypeptides that can be used to stimulate a T cell include, without limitation, CD3, CD28, inducible T cell co-stimulator (ICOS), CD137, CD2, OX40, and CD27.

This document also provides methods and materials for treating cancer. For example, one or more inhibitors of an IL-1RA polypeptide can be administered together with an adoptive cell therapy or one or more T cells (e.g., CAR T) cells having (e.g., engineered to have) a reduced level of an IL-1R1 polypeptide can be used in an adoptive cell therapy (e.g., a CAR T cell therapy) to a mammal (e.g., a human) having cancer to treat the mammal. In some cases, one or more inhibitors of an IL-1RA polypeptide can be administered together with an adoptive cell therapy to a mammal (e.g., a human) having cancer to reduce immunosuppression of the administered cells. In some cases, CAR T cells having a reduced level of an IL-1R1 polypeptide can be used in an adoptive cell therapy (e.g., a CAR T cell therapy) to a mammal (e.g., a human) having cancer to reduce T cell toxicity associated with the administered cells.

When a mammal (e.g., a human) having cancer is administered both one or more inhibitors of an IL-1RA polypeptide and an adoptive cell therapy (e.g., a CAR T cell therapy), the one or more inhibitors of an IL-1RA polypeptide can be administered before, during, and/or after the adoptive cell therapy (e.g., a CAR T cell therapy).

In some cases, methods of treating a mammal having cancer as described herein (e.g., by administering one or more inhibitors of an IL-1RA polypeptide together with an adoptive cell therapy (e.g., a CAR T cell therapy) or by administering an adoptive cell therapy including one or more T cells (e.g., CAR T) cells having (e.g., engineered to have) a reduced level of an IL-1R1 polypeptide) can be effective to reduce the size of cancer within the mammal. For example, a mammal having cancer and in need of treatment thereof can be administered one or more inhibitors of an IL-1RA polypeptide together with an adoptive cell therapy or can be administered an adoptive cell therapy including one or more T cells (e.g., CAR T) cells having (e.g., engineered to have) a reduced level of an IL-1R1 polypeptide to reduce the number of cancer cells (e.g., cancer cells expressing a tumor antigen) in the mammal by, for example, 10, 20, 30, 40, 50, 60, 70, 80, 90, 95, or more percent. In another example, a mammal having cancer and in need of treatment thereof can be administered one or more inhibitors of an IL-1RA polypeptide together with an adoptive cell therapy or can be administered an adoptive cell therapy including one or more T cells (e.g., CAR T) cells having (e.g., engineered to have) a reduced level of an IL-1R1 polypeptide to reduce the volume of one or more solid tumors (e.g., one or more tumors including cancer cells expressing a tumor antigen) in the mammal by, for example, 10, 20, 30, 40, 50, 60, 70, 80, 90, 95, or more percent.

In some cases, methods of treating a mammal having cancer as described herein (e.g., by administering one or more inhibitors of an IL-1RA polypeptide together with an adoptive cell therapy or by administering an adoptive cell therapy including one or more T cells (e.g., CAR T) cells having (e.g., engineered to have) a reduced level of an IL-1R1 polypeptide) can be effective to improve survival of the mammal. For example, a mammal having cancer and in need of treatment thereof can be administered one or more inhibitors of an IL-1RA polypeptide together with an adoptive cell therapy or can be administered an adoptive cell therapy including one or more T cells (e.g., CAR T) cells having (e.g., engineered to have) a reduced level of an IL-1R1 polypeptide to improve the survival of a mammal having cancer by, for example, 10, 20, 30, 40, 50, 60, 70, 80, 90, 95, or more percent. In another example, a mammal having cancer and in need of treatment thereof can be administered one or more inhibitors of an IL-1RA polypeptide together with an adoptive cell therapy or can be administered an adoptive cell therapy including one or more T cells (e.g., CAR T) cells having (e.g., engineered to have) a reduced level of an IL-1R1 polypeptide to improve the survival of a mammal having cancer by, for example, at least 6 months (e.g., about 6 months, about 8 months, about 10 months, about 1 year, about 1.5 years, about 2 years, about 2.5 years, about 3 years, about 4 years, about 5 years, or more).

Any appropriate amount (e.g., any appropriate dose) of one or more inhibitors of an IL-1RA polypeptide can be administered (e.g., together with an adoptive cell therapy such as a CAR T cell therapy) to a mammal (e.g., a human) having cancer. In some cases, from about 100 mg to about 10000 mg (e.g., from about 100 mg to about 8000 mg, from about 100 mg to about 6000 mg, from about 100 mg to about 4000 mg, from about 100 mg to about 3000 mg, from about 100 mg to about 2000 mg, from about 100 mg to about 1000 mg, from about 100 mg to about 500 mg, from about 500 mg to about 10000 mg, from about 1000 mg to about 10000 mg, from about 3000 mg to about 10000 mg, from about 5000 mg to about 10000 mg, from about 7000 mg to about 10000 mg, from about 8000 mg to about 10000 mg, from about 1000 mg to about 8000 mg, from about 2000 mg to about 6000 mg, from about 3000 mg to about 5000 mg, from about 500 mg to about 2000 mg, from about 1000 mg to about 3000 mg, from about 2000 mg to about 4000 mg, from about 3000 mg to about 5000 mg, from about 4000 mg to about 6000 mg, from about 5000 mg to about 7000 mg, from about 6000 mg to about 8000 mg, or from about 7000 mg to about 9000 mg) of one or more inhibitors of an IL-1RA polypeptide can be administered to a mammal having cancer to treat the mammal.

Any appropriate amount (e.g., number) of T cells (e.g., CAR T) cells (e.g., CAR T cells having (e.g., engineered to have) a reduced level of an IL-1R1 polypeptide) can be administered (e.g., in an adoptive cell therapy such as a CAR T cell therapy) to a mammal (e.g., a human) having cancer. In some cases, from about 25×10⁶ to about 1×10⁹ of T cells (e.g., CAR T) cells (e.g., CAR T cells having a reduced level of an IL-1R1 polypeptide) can be administered to a mammal having cancer to treat the mammal.

Any appropriate mammal (e.g., a human) having a cancer can be treated as described herein. Examples of mammals that can be treated as described herein include, without limitation, humans, non-human primates (e.g., monkeys), dogs, cats, horses, cows, pigs, sheep, mice, and rats. For example, a human having cancer can be administered one or more inhibitors of an IL-1RA polypeptide together with an adoptive cell therapy (e.g., a CAR T cell therapy) as described herein. For example, a human having a cancer can be treated with one or more T cells (e.g., CAR T) cells (e.g., CAR T cells having (e.g., engineered to have) a reduced level of an IL-1R1 polypeptide) in an adoptive T cell therapy (e.g., a CAR T cell therapy) as described herein.

When treating a mammal (e.g., a human) having a cancer as described herein, the cancer can be any appropriate cancer. In some cases, a cancer treated as described herein can include one or more solid tumors. In some cases, a cancer treated as described herein can be a blood cancer. In some cases, a cancer treated as described herein can be a primary cancer. In some cases, a cancer treated as described herein can be a metastatic cancer. In some cases, a cancer treated as described herein can be a refractory cancer. In some cases, a cancer treated as described herein can be a relapsed cancer. In some cases, a cancer treated as described herein can express a tumor-associated antigen (e.g., an antigenic substance produced by a cancer cell). Examples of cancers that can be treated as described herein include, without limitation, mantle cell lymphomas (MCLs), diffuse large B cell lymphomas (DLBCLs), Hodgkin's lymphomas, non-Hodgkin lymphomas, acute lymphoblastic leukemias (ALLs), chronic lymphocytic leukemias (CLLs), acute myeloid leukemias (AMLs), germ cell tumors, hepatocellular carcinomas, bowel cancers, lung cancers, breast cancers, ovarian cancers, melanomas, brain cancers, and multiple myelomas.

A cancer that can be treated as described herein can include cancer cells expressing one or more antigens. For example, a cancer that can be treated as described herein can include cancer cells that express an antigen targeted by CAR T cells provided herein (CAR T cells having (e.g., engineered to have) a reduced level of an IL-1R1 polypeptide). A cancer that can be treated as described herein can include cancer cells expressing any appropriate one or more antigens (e.g., a tumor antigen targeted by the CAR T cells). In some cases, an antigen can be a tumor-associated antigen (e.g., an antigenic substance produced by a cancer cell). In some cases, an antigen can be as listed in Table 2. Examples of tumor-associated antigens that can be targeted by an adoptive T cell therapy provided herein include, without limitation, CD19 (associated with DLBCL, ALL, and CLL), AFP (associated with germ cell tumors and/or hepatocellular carcinoma), CEA (associated with bowel cancer, lung cancer, and/or breast cancer), CA-125 (associated with ovarian cancer), MUC-1 (associated with breast cancer), ETA (associated with breast cancer), MAGE (associated with malignant melanoma), CD33 (associated with AML), CD123 (associated with AML), CLL-1 (associated with AML), E-Cadherin (associated with epithelial tumors), folate receptor alpha (associated with ovarian cancers), folate receptor feta (associated with ovarian cancers and AML), IL13R (associated with brain cancers), EGFRviii (associated with brain cancers), CD22 (associated with B cell cancers), CD20 (associated with B cell cancers), kappa light chain (associated with B cell cancers), lambda light chain (associated with B cell cancers), CD44v (associated with AML), CD45 (associated with hematological cancers), CD30 (associated with Hodgkin lymphomas and T cell lymphomas), CD5 (associated with T cell lymphomas), CD7 (associated with T cell lymphomas), CD2 (associated with T cell lymphomas), CD38 (associated with multiple myelomas and AML), BCMA (associated with multiple myelomas), CD138 (associated with multiple myelomas and AML), FAP (associated with solid tumors), CS-1 (associated with multiple myeloma), and c-Met (associated with breast cancer). For example, one or more T cells (e.g., CAR T) cells having a reduced level of an IL-1R1 polypeptide can be used in CAR T cell therapy targeting CD19 (e.g., a CART19 cell therapy) to treat cancer as described herein.

In some cases, the methods described herein can include identifying a mammal (e.g., a human) as having a cancer. Any appropriate method can be used to identify a mammal having cancer. For example, imaging techniques and biopsy techniques can be used to identify mammals (e.g., humans) having cancer.

In some cases, one or more inhibitors of an IL-1RA polypeptide administered before, during, or after an adoptive cell therapy (e.g., a CAR T cell therapy) can be administered to a mammal (e.g., a human) having cancer as the sole active agents to treat the cancer. In some cases, an adoptive cell therapy that includes one or more T cells (e.g., CAR T) cells having (e.g., engineered to have) a reduced level of an IL-1R1 polypeptide can be administered to a mammal (e.g., a human) having cancer as the sole active agents to treat the cancer.

In some cases, methods for treating a mammal (e.g., a human) as described herein (e.g., by administering one or more inhibitors of an IL-1RA polypeptide together with an adoptive cell therapy (e.g., a CAR T cell therapy) or by administering an adoptive cell therapy including one or more T cells (e.g., CAR T) cells having (e.g., engineered to have) a reduced level of an IL-1R1 polypeptide) also can include administering to the mammal one or more (e.g., one, two, three, or more) additional agents used to treat cancer and/or performing one or more (e.g., one, two, three, or more) therapies used to treat cancer. For example, a combination therapy used to treat a mammal (e.g., a human) having cancer can include administering to the mammal one or more inhibitors of an IL-1RA polypeptide together with an adoptive cell therapy (e.g., a CAR T cell therapy) or an adoptive cell therapy including one or more T cells (e.g., CAR T) cells having (e.g., engineered to have) a reduced level of an IL-1R1 polypeptide and administering to the mammal one or more (e.g., one, two, three, or more) additional agents used to treat cancer. Examples of additional anti-cancer agents that can be administered to a mammal (e.g., a human) having cancer (e.g., a cancer including one or more solid tumors) to treat the mammal include, without limitation, chemotherapeutic agents, cytotoxic agents, immune-checkpoint inhibitors (e.g., anti-PD-1 antibodies, PD-1 inhibitors, anti-PD-L1 antibodies, PD-L1 inhibitors and anti-CTLA-4 antibodies), immunomodulatory drugs (IMiDs), immune agonists (e.g., 41bb agonists), cytokines (e.g., IL-2), cancer vaccines, and any combinations thereof. In cases where one or more inhibitors of an IL-1RA polypeptide together with an adoptive cell therapy (e.g., a CAR T cell therapy) and/or an adoptive cell therapy including one or more T cells (e.g., CAR T) cells having (e.g., engineered to have) a reduced level of an IL-1R1 polypeptide are used in combination with additional anti-cancer agents, the one or more additional anti-cancer agents can be administered at the same time (e.g., in a single composition) or independently.

In some cases, a combination therapy used to treat a mammal (e.g., a human) having cancer can include administering to the mammal one or more inhibitors of an IL-1RA polypeptide together with an adoptive cell therapy (e.g., a CAR T cell therapy) or an adoptive cell therapy including one or more T cells (e.g., CAR T) cells having (e.g., engineered to have) a reduced level of an IL-1R1 polypeptide, and can include performing one or more (e.g., one, two, three, or more) therapies used to treat cancer. Examples of additional therapies that can be used to treat a mammal (e.g., a human) having cancer include, without limitation, radiation therapies, surgeries, and/or tumor-infiltrating lymphocyte (TIL) therapies. In cases where one or more inhibitors of an IL-1RA polypeptide together with an adoptive cell therapy (e.g., a CAR T cell therapy) or an adoptive cell therapy including one or more T cells (e.g., CAR T) cells having (e.g., engineered to have) a reduced level of an IL-1R1 polypeptide are used in combination with one or more therapies used to treat a mammal (e.g., a human) having cancer, the one or more additional therapies can be performed at the same time or independently of the administration of the one or more inhibitors of an IL-1RA polypeptide together with an adoptive cell therapy (e.g., a CAR T cell therapy) or the adoptive cell therapy including one or more T cells (e.g., CAR T) cells having (e.g., engineered to have) a reduced level of an IL-1R1 polypeptide. For example, one or more inhibitors of an IL-1RA polypeptide together with an adoptive cell therapy (e.g., a CAR T cell therapy) or an adoptive cell therapy including one or more T cells (e.g., CAR T) cells having (e.g., engineered to have) a reduced level of an IL-1R1 polypeptide can be administered before, during, or after the one or more additional therapies are performed.

The invention will be further described in the following examples, which do not limit the scope of the invention described in the claims.

### EXAMPLES

### Example 1: Immunosuppressive Features of Monocytes Suppress CART19 Cells

This example demonstrates that immunosuppressive cells in the tumor microenvironment (TME) such as M2-like macrophages can suppress CART19 cells functionalities. For example, immunosuppressive cells in the TME can secrete IL-1RA polypeptides, thereby diminishing IL-1 pathway signaling.

### Results

### Polarized M2-Like Macrophages Inhibit CART19 Cell Antigen-Specific Proliferation

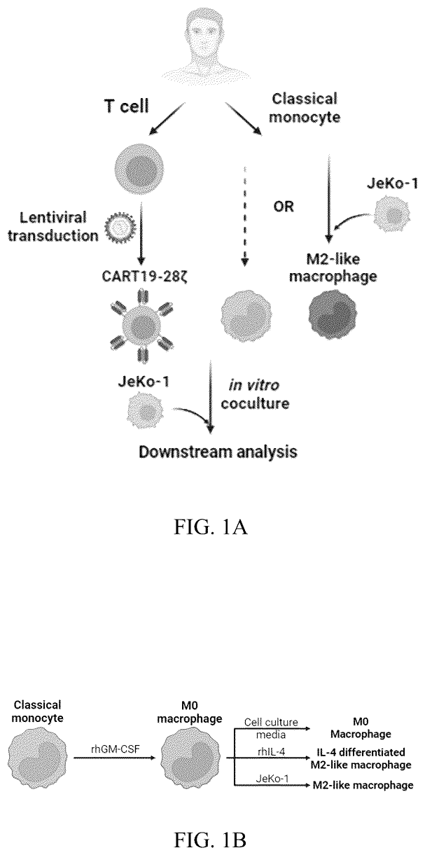

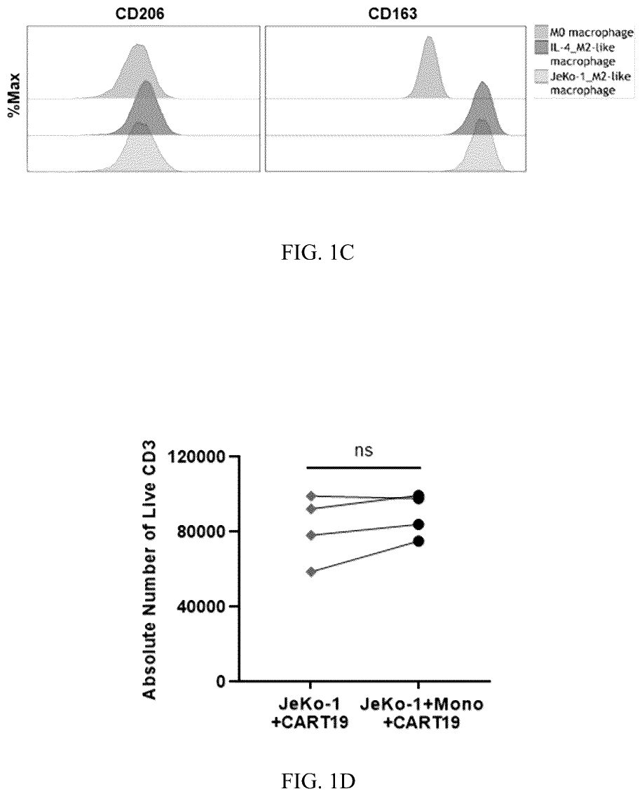

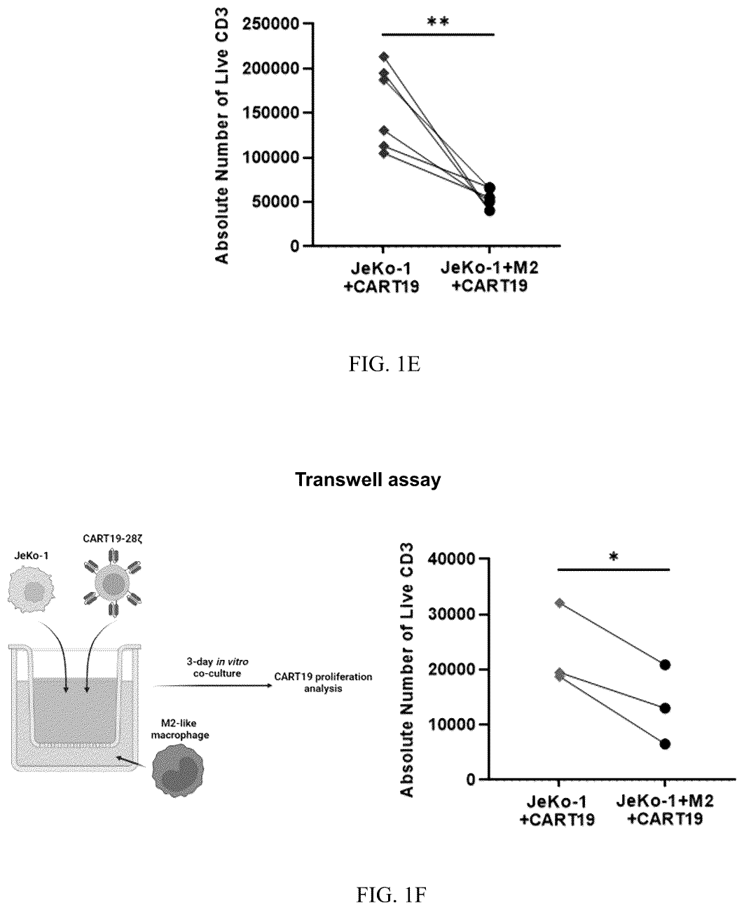

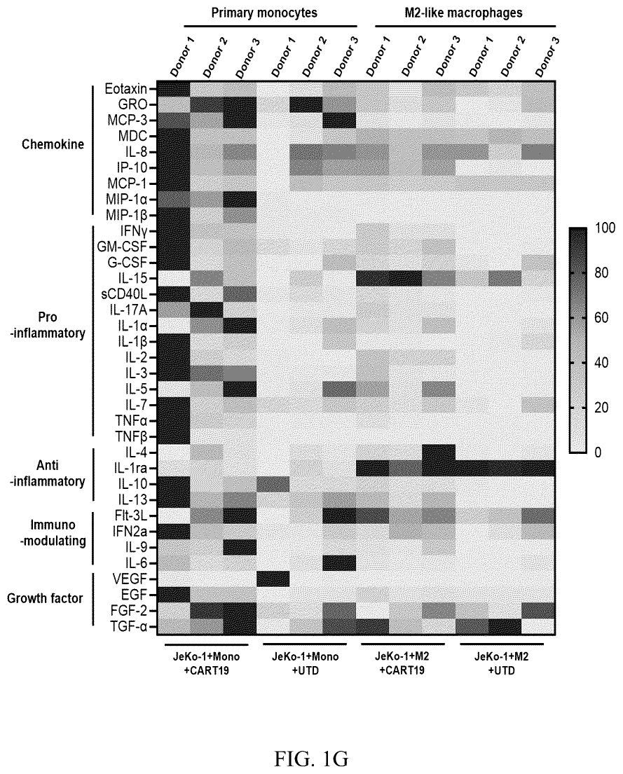

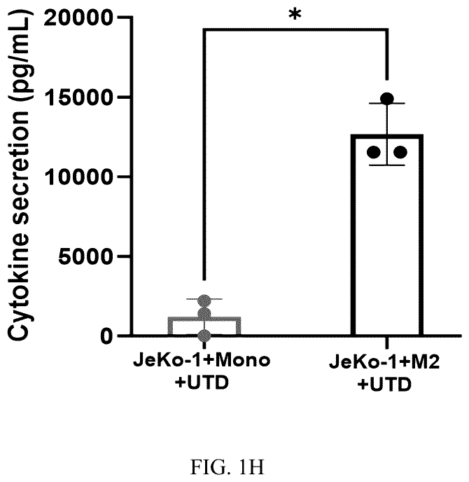

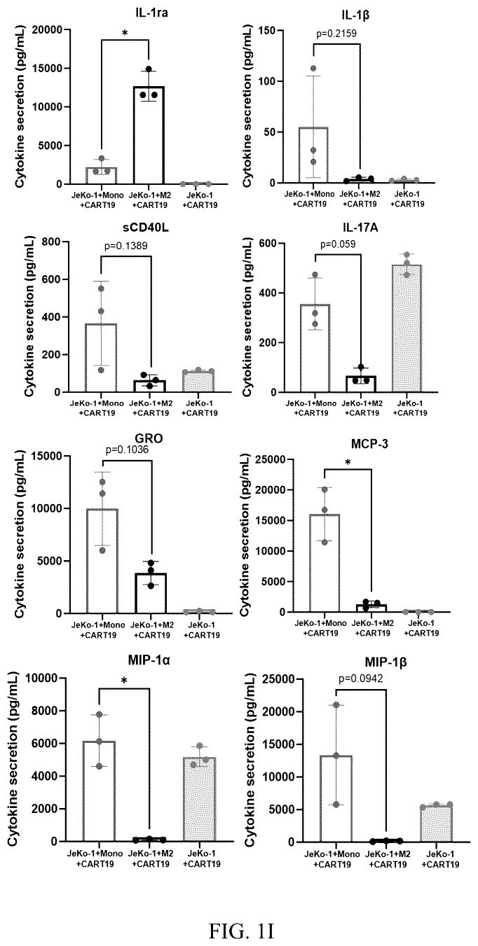

The impacts of macrophages on CART19 cell functions were studied in vitro. CD28-costimulated CART19 cells were generated from healthy donors and cocultured with JeKo-1 cells (a CD19⁺ MCL cell line) to provide antigen-specific stimulation. Freshly isolated classical monocytes or M2-polarized macrophages were added to the cocultures (FIG. 1A). To generate M2-polarized macrophages, freshly isolated monocytes were cultured in the presence of recombinant human (rh) GM-CSF for 7 days and then cocultured for 24 hours either with medium only (negative control; M0 macrophage), rh IL-4 (M2-macrophage positive control), or JeKo-1 cells (FIG. 1B). Expression of CD206 and CD163 was measured in macrophages after ex vivo polarization, confirming the M2-like phenotypes in macrophages cultured with either rh IL-4 or JeKo-1 (FIG. 1C). Next, CART19 cells, JeKo-1 cells, and fresh monocytes or ex vivo polarized M2-like macrophages were cocultured at a ratio of 2:2:1 for 3 days. CART19 cell antigen-specific proliferation was shown to be inhibited in the presence of M2-like macrophages but not in the presence of fresh monocytes (FIGS. 1D-1E).

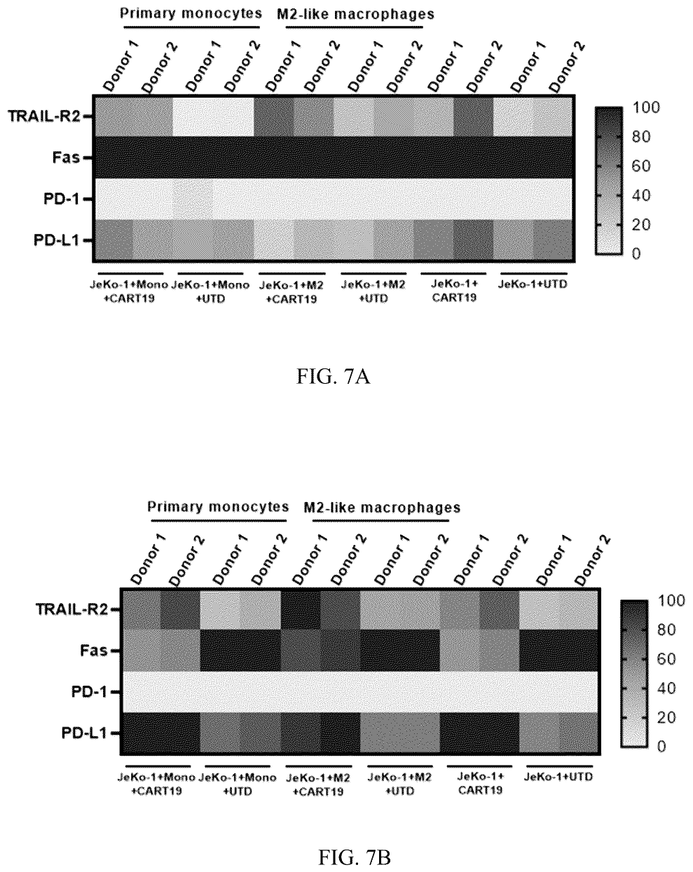

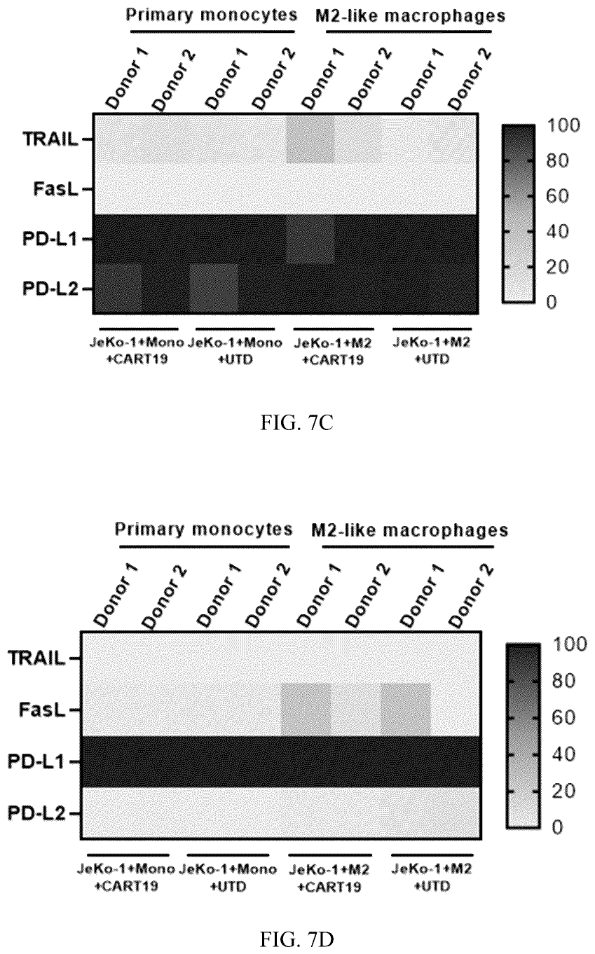

To determine the mechanisms by which M2-polarized macrophages inhibit CART19 cells in MCL, the expression of inhibitory receptors/ligands on activated macrophages and the secretion of inhibitory cytokines were studied. Cells from the cocultures (described in FIG. 1A) were harvested and assessed for the expression of TRAIL-R2, Fas, PD-1, and PD-L1 on T cells and for the expression of TRAIL, FasL, PD-L1, and PD-L2 on macrophages. There were no significant differences in the expression of these inhibitory markers between fresh monocyte and M2-like macrophage groups across multiple biological replicates (FIGS. 7A-7D). These results show that the M2-like macrophage-derived immunosuppression of CART19 cells might be mediated by soluble factors rather than inhibitory receptor/ligand interactions. To assess if this inhibition requires direct cell-cell contact, a TRANSWELL® assay was conducted where M2-like macrophages and JeKo-1-activated CART19 cells were physically separated while soluble molecules were allowed to traffic freely. In the presence of M2-like macrophages, CART19 cell proliferation was inhibited (FIG. 1F), indicating contact-independent mechanisms of suppression. To define key soluble mediators contributing to M2-mediated immunosuppression, a multiplex assay was performed on supernatants preserved from the in vitro cocultures, and 38 different chemokine and cytokines of interest were quantified (FIG. 1G). To characterize the cytokine profiles of stimulated macrophages without additional cytokines secreted from the activated CART19 cells, supernatants from cocultures of JeKo-1 cells, monocytes, and untransduced (UTD) T cells were compared to the supernatants from cocultures of JeKo-1 cells, M2-like macrophages, and UTD T cells. In the presence of M2-like macrophages, IL-1RA level was elevated (FIG. 1H). Comparing the cocultures containing activated CART19 cells with or without M2-like macrophages revealed increased secretion of IL-1RA and reduced secretion of multiple chemokines and proinflammatory cytokines, including GRO, MCP-3, sCD40L, IL-17A, IL-1β, MIP-1α, and MIP-1β, when M2-like macrophages were present (FIG. 1I). 1RA

### Downregulated IL-1β Signaling and its Downstream Pathways in Baseline CART19 Cells

To test if IL-1RA contributes to M2-like macrophage mediated CART19 cell inhibition in MCL cells, single cell RNA sequencing (scRNAseq) was performed on cryopreserved baseline autologous brexu-cel products from MCL patients treated with brexu-cel, where 4 patients did not respond (stable or progressive disease), and 3 patients subsequently relapsed (Wang et al., New. Engl. Journ. Of. Med. 382:1331-1342 (2020)). ScRNAseq was performed on the baseline products from these 7 patients as well as from 5 matched responders who achieved complete remission. Specifically, IL-1 pathway signaling in non-responders was compared to initial responders (responders and relapsed). The scRNAseq analysis identified CD4⁺ CART19 cell populations, heat shock protein (HSP)-high CD8⁺ memory CART19 cell populations, proliferative CD8⁺ CART19 cell populations, activated CD8⁺ CART19 cell populations, and cytotoxic CD8⁺ CART19 cell populations in the brexu-cel products of these patients (FIG. 2A). In total, 90,177 cells were sequenced from responder samples, relapsed samples, and non-responder samples. All downstream RNA expression analyses were conducted based on comparisons between non-responders (42,836 cells) and initial responders (47,341 cells) within each cell population of interest. Pathway analysis demonstrated downregulation of IL-1β-induced signaling pathways (e.g., NF-κB and stress-activated MAPK cascade (p38)) in CAR T cell populations of non-responders (FIG. 2B). CD4⁺ CART19 cells from the non-responders showed reduced T-helper 17 (Th17) type immune responses and differentiation (FIG. 2B), implying impaired IL-1β signaling in the non-responders. Additionally, T cell activation and proliferation were reduced in non-responder CAR T cells (FIG. 2B). Collectively these data highlight the downregulated IL-1 signaling in brexu-cel products (prior to infusion) in non-responders.

### M2-Like Phenotypes and Downregulation of IL-1β Pathways in Peripheral Monocytes

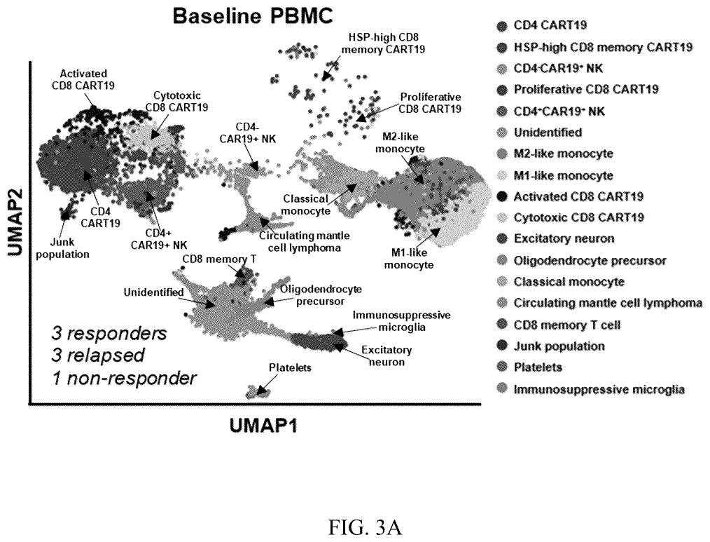

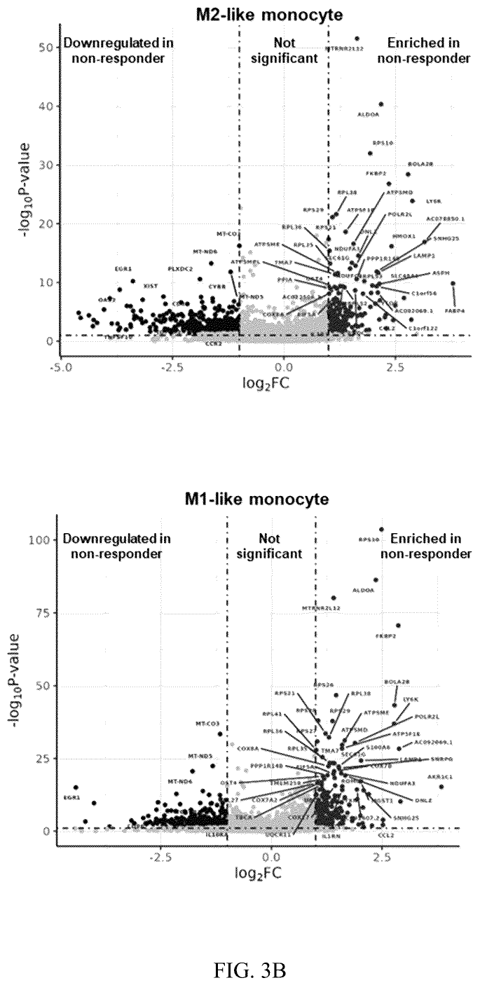

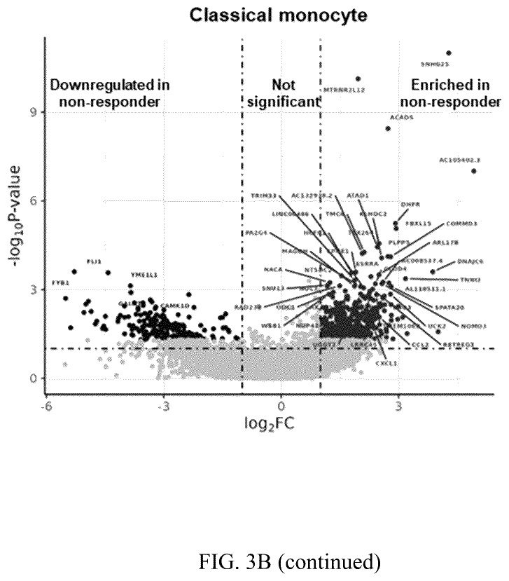

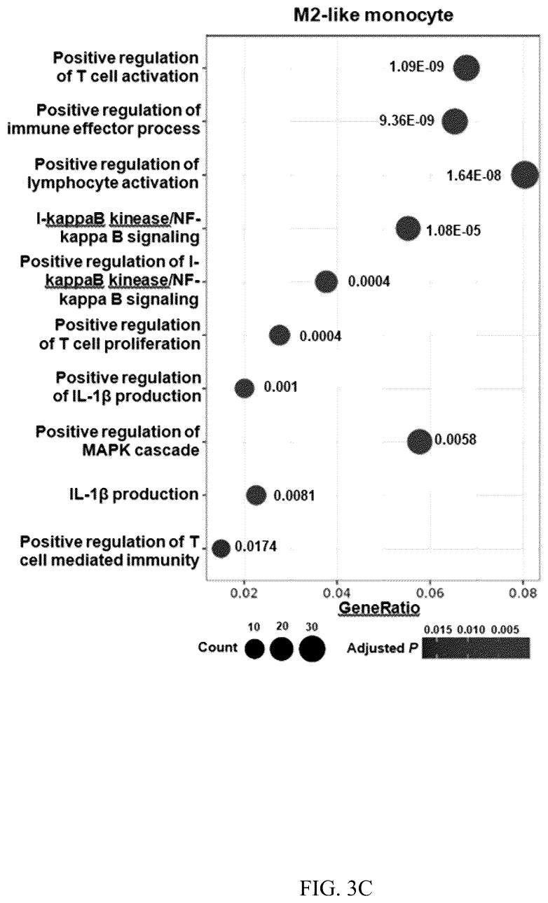

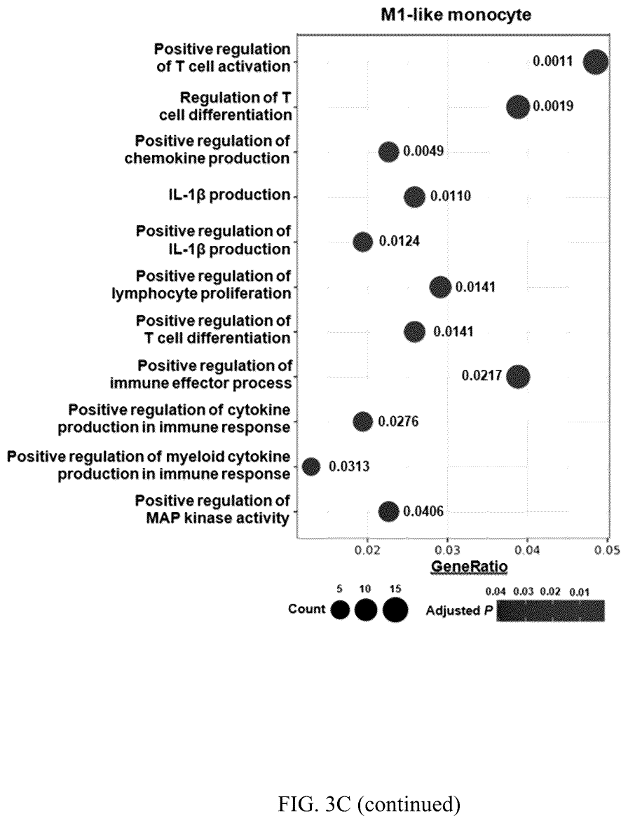

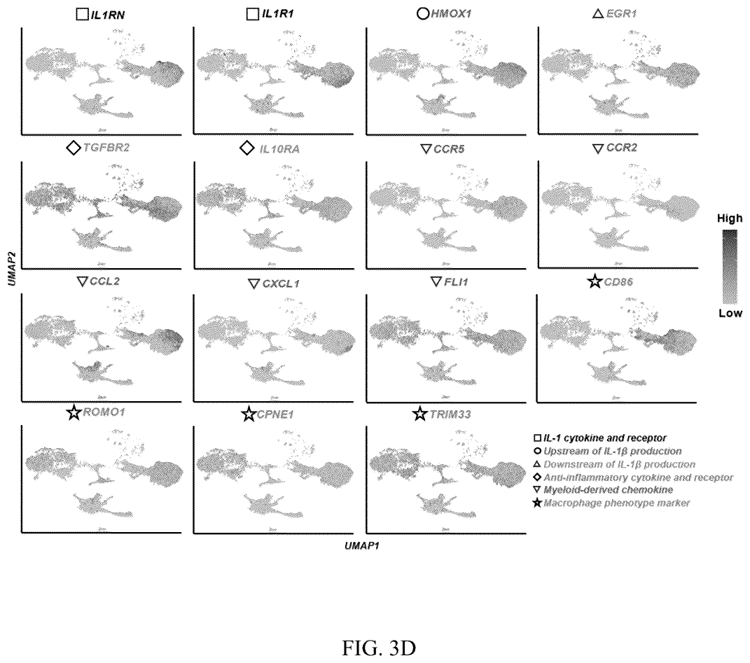

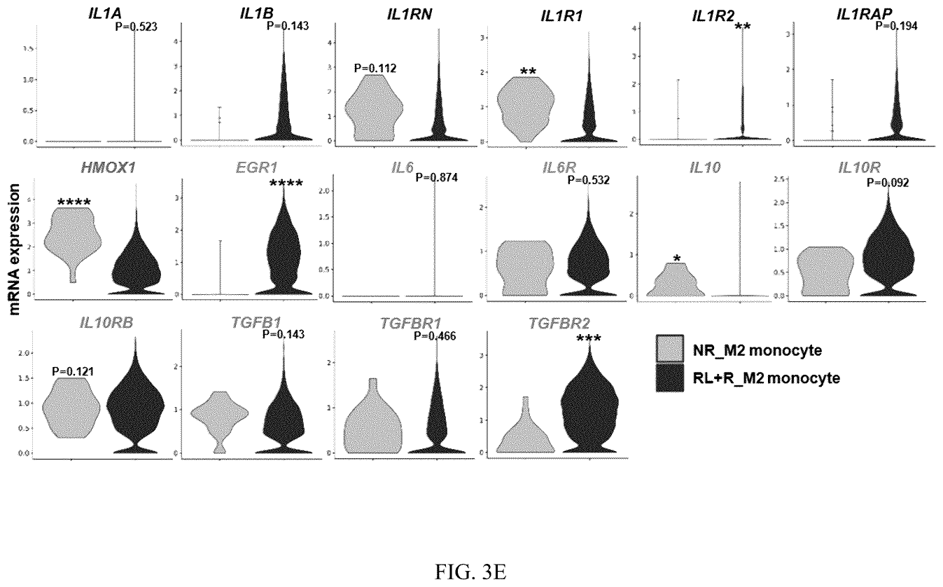

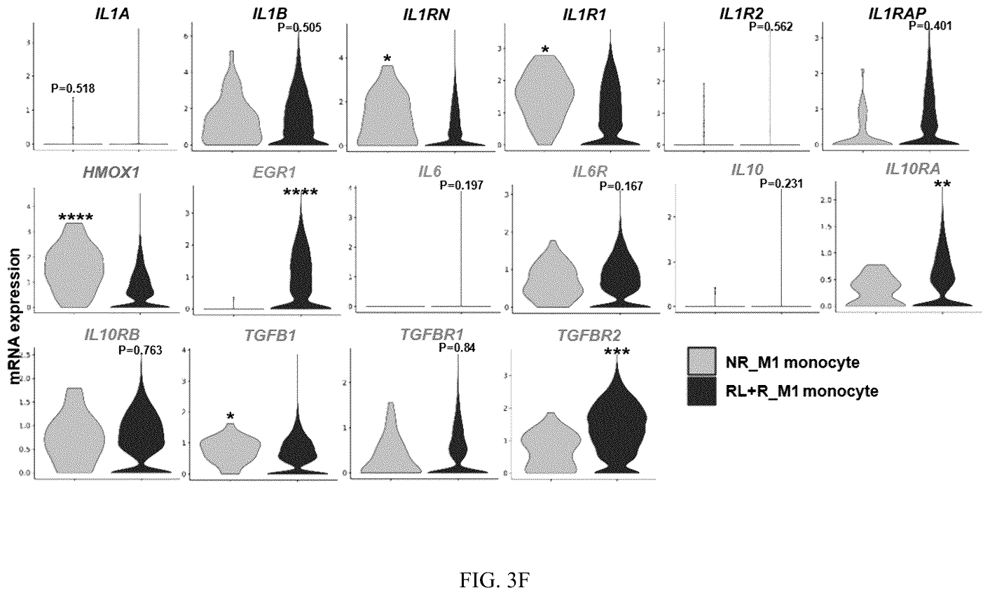

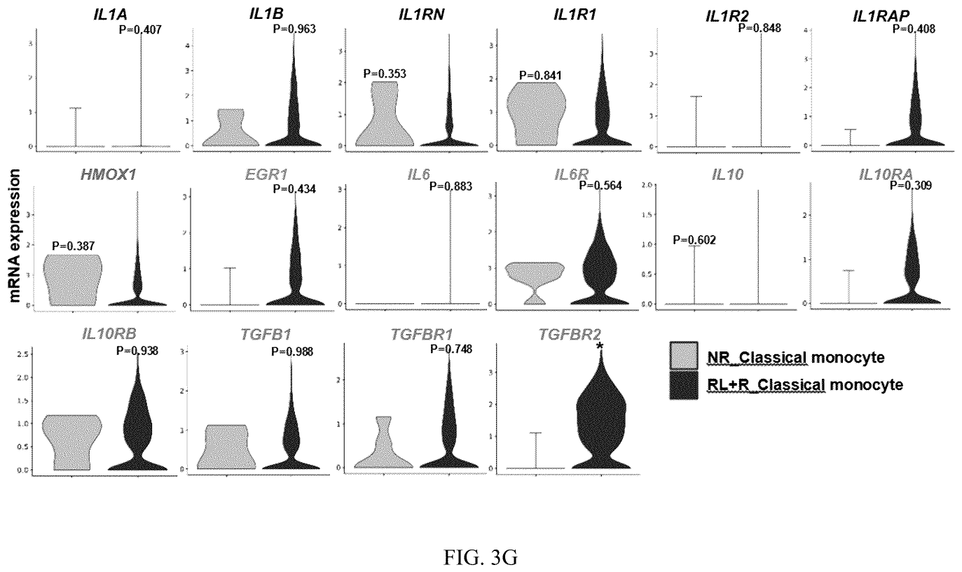

To test if the downregulation of IL-1 signaling in the brexu-cel products was associated with a specific myeloid cell signature at baseline in the non-responders, the cryopreserved autologous baseline peripheral blood mononuclear cells (PBMC) samples from the same cohort of MCL patients treated with brexu-cel, and whose brexu-cel products were sequenced in FIG. 2 were further analyzed. The cryopreserved baseline PBMCs (prior to brexu-cel treatment) from these 12 patients (5 responders, 3 relapsed, and 4 non-responders) were recovered and prepared for scRNAseq analysis. Samples from 3 responders, 3 relapsed, and 1 non-responder were qualified for downstream RNA analysis according to quality control screening. The scRNAseq data analysis identified monocyte populations, including M2-like monocytes, M1-like monocytes and classical monocytes, in the PBMCs (FIG. 3A). In total, 41,002 cells were sequenced from responder samples, relapsed samples, and non-responder samples. All downstream RNA expression analyses were conducted based on comparisons between non-responders (871 cells) and initial responders (40,131 cells) within each cell population of interest.

Differential gene expression analysis in M2-like monocytes from the non-responders revealed enrichment of HMOX1 expression and downregulation of EGR1 expression as compared to the initial responders (FIG. 3B). Pathway analysis demonstrated downregulation of pathways involved in IL-1β production (e.g., MAPK and NF-κB signaling cascade) and T cell-mediated immunity in M2-like monocytes within the non-responders (FIG. 3C), suggesting immunosuppressive phenotypes and functions, as well as impaired IL-1β production in the non-responders' M2-like monocytes. Additionally, the M2-like monocytes from the non-responder population exhibited a higher expression level of CCL2, a gene encoding chemoattractant MCP-1 and IL-1R1, and lower expression levels of CD86, CCR5, CCR2 (FIG. 3B).

Analysis of the differentially expressed genes and pathways of the M1-like monocyte population identified enrichment of IL1RN expression1RA and downregulation of EGR1 expression in the non-responders (FIG. 3B). Consistent with the observations made in the M2-like monocyte population, the corresponding pathway analysis indicated decreased IL-1β production and reduced positive regulation of T cell functions as well as cytokine/chemokine production in the M1-like monocytes from the non-responders (FIG. 3C). Similarly, higher expression levels of CCL2 and ROMO1, and downregulation of CD86 expression, TGFBR2 expression, and IL10RA expression, was observed in the non-responders (FIG. 3B). Collectively, these data suggest that M1-like monocyte population in the non-responders suppressed IL-1β signaling.

A subsequent analysis of differential gene expression and signaling pathways of the classical monocyte population detected upregulation of CPNE1 expression and TRIM33 expression in the non-responders (FIG. 3B). The analysis also revealed enrichment of CXCL1 expression in the non-responders (FIG. 3B), as well as decreased expression of FLI1 and of TGFBR2 (FIG. 3B). These data suggest that classical monocytes of the non-responders could differentiate and acquire immunosuppressive phenotypes thus inhibiting T cell immunity.

Expression of selected genes identified from differential gene expression analyses were projected onto the UMAP based on scRNAseq data. Enrichment of HMOX1 expression and IL1RN expression was mainly detected in the M2-like monocyte cluster, while CXCL1 expression and IL1R1 expression were relatively enriched in the M1-like monocyte population (FIG. 3D). TGFBR2 expression was upregulated in the M1-like monocyte population, which indicated potential for being reprogrammed to acquire immunosuppressive phenotypes due to TGF-β signaling (FIG. 3D). Enriched CCR2 expression was observed in the M1-like monocyte population, suggesting a stronger responsiveness to proinflammatory features of MCP-1 and MCP-3 than the M2-like monocytes (FIG. 3D). Furthermore, CCL2 expression was more enriched in the M2-like monocytes than the M1 population in the selected patients (FIG. 3D), which was surprising as M1 macrophages have been shown to produce high MCP-1 (Chen et al., Cell. Death. Disc. 7: e2115-e2115 (2016)). CD86 did not show significant elevation in M1-like monocytes versus M2-like populations (FIG. 3D). ROMO1, EGR1, CCR5, IL10RA, CPNE1, TRIM33, and FLI1 genes were not preferentially expressed in M2-like monocyte, M1-like monocyte, or classical monocyte populations (FIG. 3D).

### Il-1-Associated Cytokines

Violin plots were generated for mRNA expression of IL1A, IL1B, IL1RN, IL1R1, IL1R2, IL1RAP, IL6, IL6R, IL10, IL10RA, IL10RB, TGFB1, TGFBR1, and TGFBR2 in each population of interest (FIGS. 3E-3G). Trends of elevated expression of IL1B and reduced expression of IL1RN were observed in the initial responders in all three cell populations of interest (FIGS. 3E-3G), which was consistent with the pathway analysis. IL1R1 expression showed increasing trends while ILIR2 and IL1RAP showed downregulation in all clusters of interest in the non-responders (FIGS. 3E-3G). IL6 expression as well as IL6R expression, were elevated in the initial responders, demonstrating an upregulated IL-1β signaling in these patients compared to the non-responders (FIGS. 3E-3G). IL10 expression was more enriched in the non-responders compared to the responder and relapsed groups within the M2-like monocyte and classical monocyte populations (FIG. 3E and FIG. 3G). Its cognate receptor subunit, IL10RA, was downregulated in the non-responders across all the cell populations of interest, and IL10RB showed similar trends, although the same was not observed in the M2-like monocyte cluster (FIGS. 3E-3G). TGFB1 expression was elevated in the initial responders in the M2-like monocyte and classical monocyte populations but decreased in the M1-like monocyte cluster (FIGS. 3E-3G). TGF-β receptors, including TGFBR1 and TGFBR2, were upregulated in the initial responders across all the cell populations of interest (FIGS. 3E-3G).

Altogether, the single cell sequencing analyses of peripheral monocytes from the non-responders showed upregulation of genes involved in M2-like differentiation and IL1RN, as well as downregulation of IL-1β production and T cell mediated immunity pathways. The in vitro cocultures described in FIG. 1 indicate that M2-like macrophages inhibited T cell proliferation in MCL and secreted high level of IL-1RA.

### Neutralizing IL-1RA Improved CART19 Cell Effector Functions

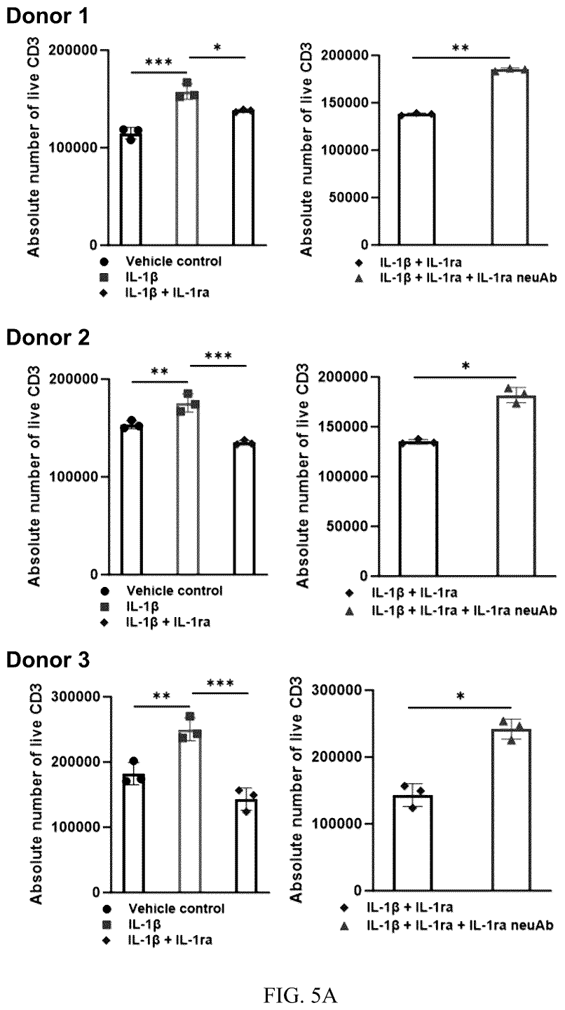

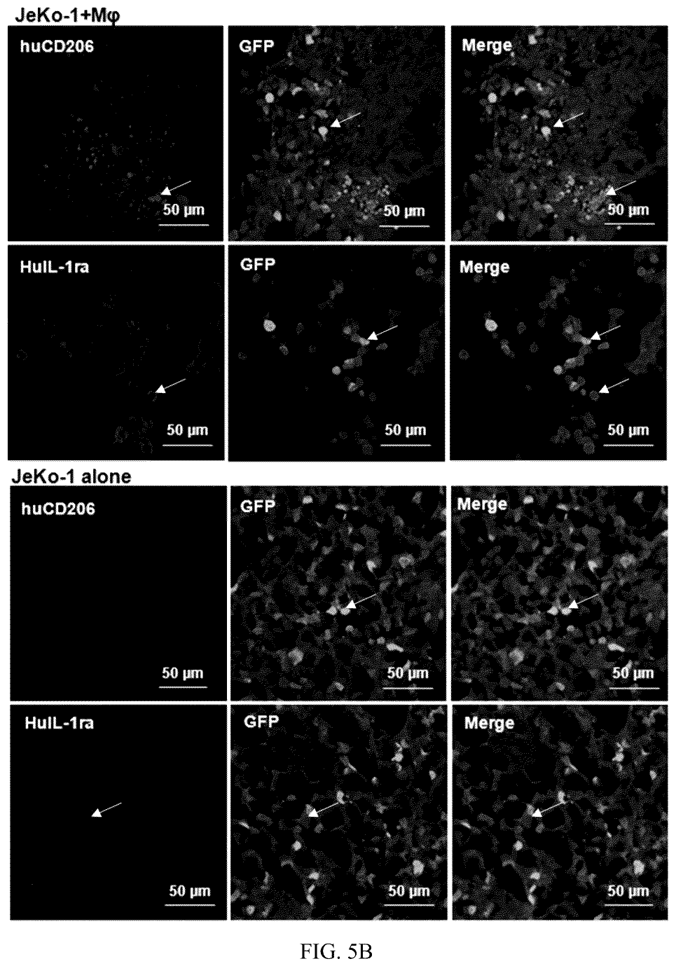

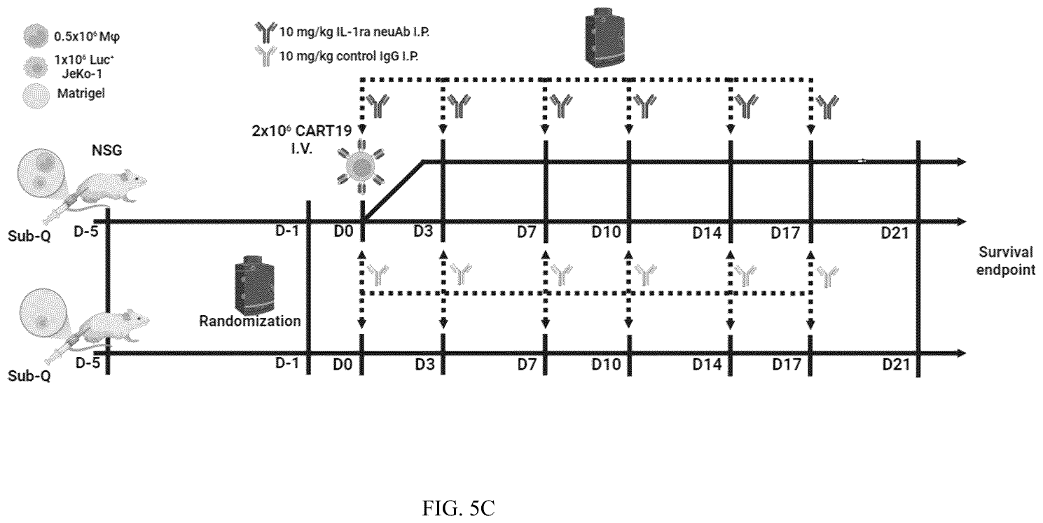

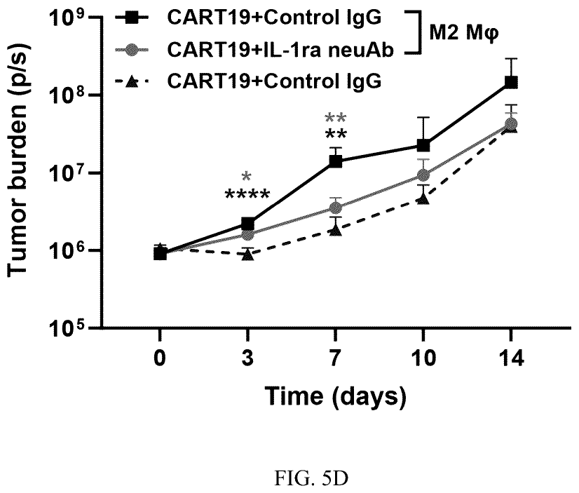

The functions and activities of IL-1RA were investigated in vitro. To determine if M2-like macrophages modulate IL-1RA production in immune cells other than M2-like monocytes, JeKo-1 cells, UTD T cells, and fresh monocytes or ex vivo polarized M2-like macrophages were cocultured for 3 days as shown in FIG. 1A. At the end of the coculture, IL-1RA expression in JeKo-1 cells, monocytes, M2-like macrophages, and UTD T cells was analyzed by flow cytometry. JeKo-1 cells were confirmed to express IL-1RA intracellularly (FIG. 4D). The results showed that M2-like macrophages were the main producer of IL-1RA compared to the monocytes (FIG. 4A), and that the presence of M2-like macrophages led to increased IL-1RA expression in both T cells (FIG. 4B) and tumor cells (FIG. 4C), suggesting that M2-like macrophages have the potential to modulate surrounding cells to become more immunosuppressive. To study the effect of IL-1RA on CART19 function directly, CART19 cells were stimulated with JeKo-1 cells and the media was supplemented with recombinant human (rh) IL-1β alone, IL-1β combined with IL-1RA, or vehicle control for 3 days. The T cell proliferation assay demonstrated IL-1β enhanced CART19 cell antigen-specific proliferation, which was then inhibited by IL-1RA (FIG. 5A), implying that IL-1RA suppressed IL-1β-dependent CART19 proliferation. An IL-1RA neutralization assay was performed which showed that blocking IL-1RA with a monoclonal antibody preserved the IL-1β-enhanced CAR T cell proliferation (FIG. 5A). Next, the impact of M2-like macrophage-derived IL-1RA was tested in a MCL xenograft mouse model. To confirm the M2 phenotypes of the engrafted macrophages and IL-1RA production, M0 macrophages and luciferase⁺ JeKo-1 cells were resuspended in MATRIGEL® at a ratio of 1:2 and were subcutaneously engrafted into NOD-SCID-γ^{-/-} (NSG) mice. After 7 days, tumor tissues were dissected and prepared for immunofluorescence imaging. The engrafted M0 macrophages developed M2-like phenotypes (human CD206⁺) (FIG. 5B). Notably, as shown in FIG. 4A and FIG. 4C, both JeKo-1 cells and M2-like macrophages expressed human IL-1RA, but consistent with the in vitro data (FIG. 4C), tumors containing macrophages and JeKo-1 cells showed elevated IL-1RA signals compared to tumors with JeKo-1 cells alone (FIG. 5B). After validating M2-like macrophage polarization in vivo, therapeutic effects of IL-1RA neutralizing antibody on CART19 efficacy was evaluated in this model. NSG mice were subcutaneously engrafted with either 1×10⁶ luciferase⁺ JeKo-1 cells plus 5×10⁵ M0 macrophages or 1×10⁶ luciferase⁺ JeKo-1 cells alone in MATRIGEL®. After confirming tumor engraftment with bioluminescence imaging (BLI), mice were randomized to treatments either with an intravenous injection of CART19 cells in combination with 10 mg/kg IL-1RA neutralizing antibody or with an intraperitoneal injection of control IgG (twice weekly for three weeks). Tumor burden was assessed by serial BLI, and all mice were alive when the survival endpoint of the study was reached (120 days) (FIG. 5C). Delayed tumor progression was observed in mice engrafted with macrophages and treated with IL-1RA neutralizing antibody compared to mice engrafted with macrophages and treated with control IgG, indicating that neutralizing IL-1RA diminished the tumor-promoting effects from engrafted macrophages (FIG. 5D). In addition, in the IgG control-treated groups, mice engrafted with macrophages exhibited faster tumor growth than mice engrafted with JeKo-1 cells alone (FIG. 5D), further demonstrating that M2-like macrophages promoted tumor growth.

### IL1-Ra Inhibition of IL-Iβ Mediated CART19 Cell Proliferation

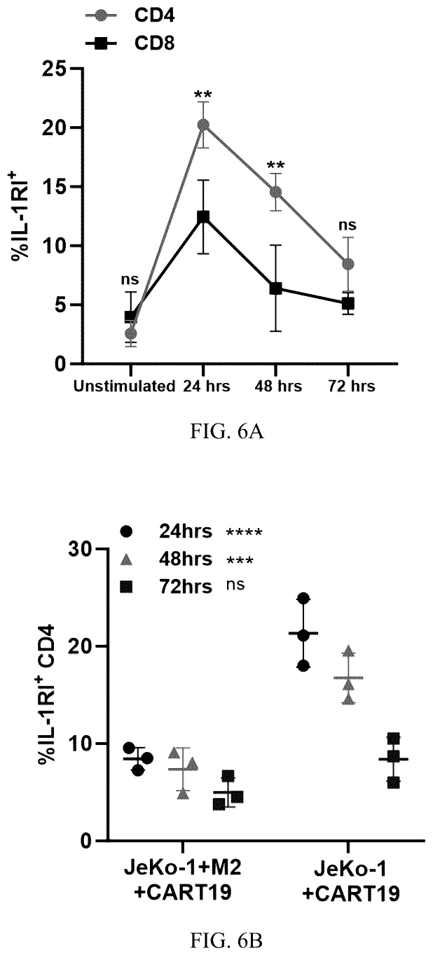

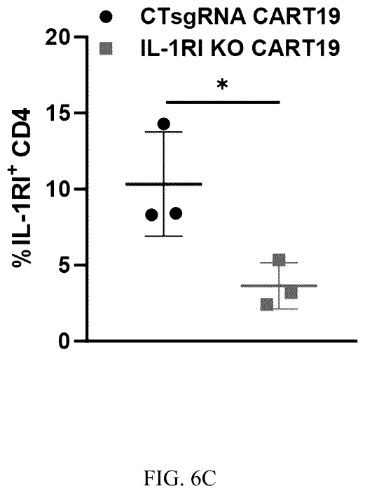

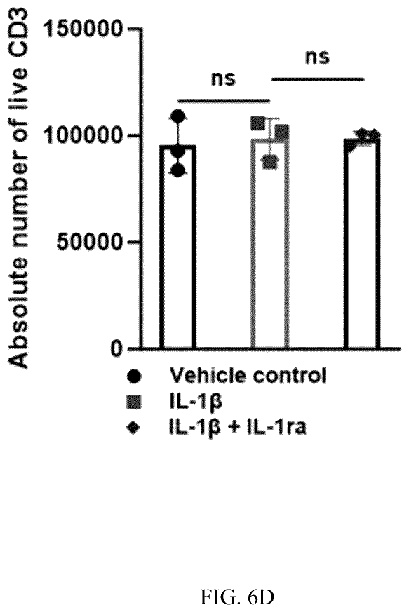

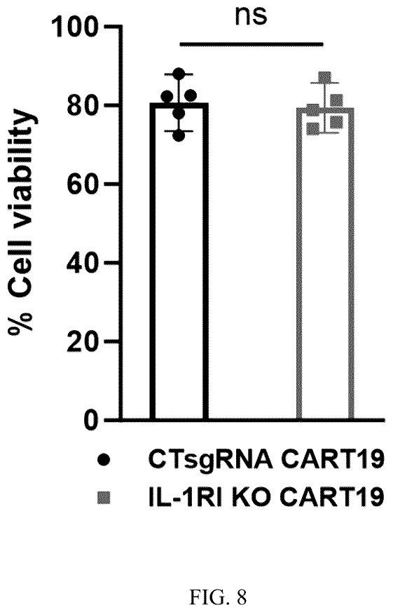

To analyze how M2-like macrophages interact with the IL-1 receptors on CART19 cells, the expression of IL-1 receptor 1 (IL-1R1), the cognate receptor of both IL-1RA and IL-1β, was evaluated on T cells. CART19 cells and JeKo-1 cells were cocultured at a 1:1 ratio for 3 days, during which IL-1R1 expression was analyzed on both CD4⁺ and CD8⁺ T cells daily. IL-1R1 expression was enhanced in stimulated T cells, predominantly found on CD4⁺ T cells, and peaked 24 hours after initiation of coculture (FIG. 6A). Next, M2-like macrophages were incorporated into the cocultures as described in FIG. 1A. The presence of M2-like macrophages induced downregulation of IL-1R1 on JeKo-1 cell-stimulated CAR T cells (FIG. 6A). To determine if IL1-ra inhibits CART19 cells by binding to IL-1R1, IL-1R1 knockout (KO) CART19 cells (IL-1R1^{k/o} CART19 cells) were generated by lentiviral transduction of a CRISPR/Cas9 system. IL-1R1^{k/o} CART19 cells did not reduce cell viability during CAR T cell production (FIG. 8), demonstrating that IL-1R1 expression did not affect T cell survival. Knockout efficiency was confirmed by flow cytometry (FIG. 6C). To study the response of IL-1R1^{k/o} CART19 cells to IL-1, engineered CART19 cells were cocultured with JeKo-1 cells supplemented with rhIL-1β and IL-1RA for 3 days. CAR T cell antigen-specific proliferation was not altered by IL-1 stimulation (FIG. 6D). These data indicate that both IL-1β and IL-1RA effects were mediated by their interactions with the IL-1R1 on stimulated CART19 cells.

### Materials and Methods

### Cell Lines

All cell lines were maintained at 37° C. and 5% CO₂. JeKo-1 cells, a CD19⁺ mantle cell lymphoma (MCL) cell line, was purchased from ATCC (CRL-3006, Manassas, VA, USA). Luciferase⁺ JeKo-1 cells were generated by transducing Jeko-1 cells with luciferase-ZsGreen lentivirus (Addgene, Cambridge, MA, USA) and sorted to 100% purity. Cell lines were maintained in R20 medium composed of RPMI 1640 (Gibco, 11875093, Gaithersburg, MD, USA), 20% FBS (Corning Life Sciences, 35-011-CV, Corning, NY, USA), and 1% penicillin-streptomycin-glutamine (Gibco, 10-378-016).

### Monocyte Isolation and Ex Vivo M2-Like Macrophage Polarization

PBMCs were isolated from healthy donor apheresis cones by density gradient centrifugation and resuspended in 2% FBS in PBS (Gibco). Human classical monocytes were isolated from fresh PBMCs by classical monocyte isolation kit (Miltenyi Biotec, 130-117-337, Bergisch Gladback, Germany) according to the manufacturer's instructions. All in vitro and ex vivo cultures were maintained in T cell medium (TCM) containing X-VIVO 15 (Lonza, 02-060Q, Walkersville, MD, USA), 10% heat-inactivated human AB serum plasma (Innovative Research, Inc., 44277, Novi, MI, USA), and 1% penicillin-streptomycin-glutamine. Purity (>90% CD14⁺ CD16⁻) was confirmed by flow cytometry. To differentiate classical monocytes into M0 macrophages, freshly isolated monocytes were resuspended in TCM at a concentration of 1×10⁶/mL and incubated with 10 ng/ml of rhGM-CSF (STEMCELL, 78015, Vancouver, Canada) at 37° C. for 7 days as described elsewhere (Ruella et al., Can. Disc. 7:1154-1167 (2017)). To generate M2-like macrophages, M0 macrophages were incubated in TCM with either 20 ng/mL rhIL-4 (STEMCELL, 78045) or JeKo-1 cells at a ratio of 1:2 for another 24 hours. Differentiated macrophages were detached from tissue culture plates by incubating on ice for at least 30 minutes and pipetting every 10 minutes with ice cold PBS. Adherence of residual cells was checked under a microscope until all the macrophages were detached. M2 phenotype was confirmed by flow cytometry (CD14⁺ CD163^{high} CD206^{high}). M0 macrophages were used as a baseline control to distinguish M2-like phenotypes in differentiated macrophages.

### Cart19 Cells and Monocytes or Macrophages

CART19-28ζ cells were generated from healthy donor PBMCs as described elsewhere (Sakemura et al., Blood, 139 (26): 3708-3721 (2022)). Freshly isolated monocytes or M2-like macrophages were cocultured with CART19 cells and JeKo-1 cells at a ratio of 1:2:2 at 37° C. Cells were collected on day 3 and were assessed for CART19 cell antigen-specific proliferation by absolute counts of CD3⁺ cells as well as by expression of surface markers of interest on both T cells (CD3, PD-1, PD-L1, Fas, and TRAIL-R2) and monocytes (CD14, TRAIL, FasL, PD-L1, and PD-L2). Supernatants of cocultures were also collected and cryopreserved at −80° C. for cytokine analysis. Cytokines of interest were quantified by MILLIPLEX® MAP Human Cytokine/Chemokine Magnetic Bead Panel (Millipore Sigma, HCYTMAG-60K-PX38, Ontario, Canada) according to the manufacturer's instructions.

For TRANSWELL® assays, M2-like macrophages were generated as previously described (Ruella et al., Can. Disc. 7:1154-1167 (2017)) and seeded in the bottom chamber of a TRANSWELL® plate (0.4 μm). JeKo-1 cells and CART19 cells were added at a ratio of 1:1 into the upper wells on day 8 of the M2-like macrophage differentiation timeline. Cells were cocultured at 37° C. for an additional 3 days. On day 11, cells in the upper chambers were harvested and washed with PBS for subsequent analysis.

### Single-Cell RNA Sequencing

### Sample Preparation

Cryopreserved patient PBMCs and autologous brexu-cel products from 5 responders, 3 relapsed, and 4 non-responders from the ZUMA-2 clinical trial (Wang et al., New. Eng. Jour. Of. Med., 382:1331-1342 (2020)) were carefully thawed and washed once with TCM. Cells were resuspended in 0.04% human AB serum in PBS at a concentration of >1×10⁵ cells/mL. Due to low cell count/poor cell quality, PBMCs from 3 responders, 3 relapsed, and 1 non-responder were qualified for downstream RNA sequencing analysis. Brexu-cel samples from all patients were processed for downstream RNA sequencing analysis.

### Mapping

The single cell sequence preprocessing was performed using the standard 10× Genomics Cell Ranger Single Cell Software Suite. Raw reads were aligned to the hg38 reference genome. UMI (unique molecular identifier) counting was performed using Cell Ranger v7.1.0 pipeline with default parameters.

### Quality Control, Integration, Clustering, and Differential Expression Analysis

Seurat_4.9.9 was used for all following analyses. All genes that were not detected in at least three single cells were excluded. Cells in which fewer than 200 unique genes were detected were excluded to remove possible low-quality cells. Mitochondrial quality control metrics were calculated using the ‘PercentageFeatureSet’ function to filter out cells with >40% mitochondrial counts to exclude low-quality and dying cells. Normalization was performed by the global-scaling normalization method ‘LogNormalize’ in Seurat, and then the results were log-transformed for downstream analysis. Then the ‘FindVariableFeatures’ function was used to calculate a subset of highly variable features (default: 3000 genes) for each sample to highlight biological signals in future analyses. A linear transformation as a standard pre-processing step was performed on the combined dataset. Then, Harmony R package was applied to integration analysis for batch effect removal and primary clustering. Principal component analysis was conducted to determine the dimensionality of the dataset, and UMAP was used to visualize the combined dataset. The ‘FindNeighbors’ and ‘FindClusters’ functions were applied to cluster cells, cluster-specific markers conserved across conditions were identified with the ‘FindConservedMarkers’ function, and clusters were assigned to known cell types based on their specific markers. After all the cells were aligned, comparative analysis was performed to identify differentially expressed genes (DEGs) induced by different conditions. Genes with P value <0.05 and |log₂fold change|>1 were defined as significant DEGs. All marker genes and DEGs were visualized with ‘FeaturePlot’ and ‘VInPlot’ functions in Seurat and the R package ‘ggplot2’. R package ‘clusterProfiler’ was applied for DEG set enrichment analysis and visualization. Only functional categories related to IL-1 pathway and T cell immunity were chosen.

### Il-1RA Intracellular Staining

JeKo-1 and untransduced T cells were cocultured with freshly isolated monocytes at a ratio of 2:2:1 at 37° C. overnight. After 16-18 hours, 1000X brefeldin A (eBioscience, 00-4506-51, San Diego, CA, USA) was diluted in TCM and directly added to the cocultures, after which cells were incubated for another 24 hours. Similarly, cells from M2-like macrophage assays were cocultured for 3 days before 1X brefeldin A was added. All cells were harvested on ice and then fixed and permeabilized by FIX & PERM Cell Permeabilization Kit according to the manufacturer's instructions (Invitrogen, GAS004, Carlsbad, CA, USA). Cells were incubated with anti-human IL-1RA antibody (ThermoFisher, 11-7015-82) at room temperature in the dark for 30 minutes. Expression of IL-1RA was then measured by flow cytometry.

In vitro functional assay of IL-1β and IL-1RA

CART19 cells resuspended at 1×10⁶ cells/mL were cocultured with JeKo-1 at a ratio of 1:1 for 3 days. 100 μg/mL rhIL-1β (R&D systems, 201-LB-010), 100 ng/mL rhIL-1RA (R&D systems, 280-RA-010), or 20 μg/mL IL-1RA neutralizing monoclonal antibody (clone 10309, Invitrogen, MA5-23802) were added at the beginning of the coculture according to the indicated experimental conditions. On day 3, cells were harvested and processed for CAR T cell proliferation assay.

### Mantle Cell Lymphoma Xenograft Mouse Model

Male and female 6-8 week-old NOD-SCID-γ^{-/-} (NSG) mice were housed in an animal barrier space for BSL2⁺ level experiments.

Human M0 macrophages were differentiated in vitro as described elsewhere (Ruella et al., Cancer Discov., 7:1154-1167 (2017)). Cells were carefully detached and harvested on ice and washed by cold PBS, prior to resuspension in PBS to reach a final concentration of 1×10⁷ cells/mL. Luciferase⁺ JeKo-1 cells were washed with cold PBS and resuspended to reach 2×10⁷ cells/mL. Macrophages and luciferase⁺ JeKo-1 cells were thoroughly mixed at a 1:2 ratio. For mice that received JeKo-1 cells alone, tumor cells were mixed with same volume of PBS instead. Prepared cells (100 μL) were mixed with 100 μL of MATRIGEL® (Corning, CLS354230) in a 1 mL tuberculin syringe. MATRIGEL® was thawed on ice overnight at 4° C. the day before tumor injection. Tumor cells were subcutaneously injected to the right flank of each mouse. Tumor burden was measured based on BLI by Xenogen IVIS-200 Spectrum camera (PerkinElmer, Hopkinton, MA, USA). Imaging was performed 10 minutes after intraperitoneal injection of 10 μL/g D-luciferin (15 mg/mL, Gold Biotechnology, St. Louis, MO, USA). Once tumor burden reached 10⁶ photons/second, mice were randomized according to tumor burden. All mice were treated with 2×10⁶ CART19 cells intravenously by tail vein injection. In the indicated experiments, mice received 10 mg/kg of either IL-1RA neutralizing antibody (clone 10309, Invitrogen, MA5-23802) or IgG control (clone C1.18.4, BioXCell, BE0085) intraperitoneally twice a week for three weeks. Serial BLI was performed to assess tumor burden.

### Immunofluorescence Microscopy

Human M0 macrophages were prepared and resuspended in PBS at a concentration of 1×10⁸ cells/mL. Luciferase⁺ JeKo-1 cells were prepared and resuspended in PBS at 2×10⁸ cells/mL. JeKo-1 cells were prepared with macrophages or PBS alone at a 1:1 volume ratio. Prepared cells (100 μL) were mixed with the same volume of MATRIGEL®, followed by subcutaneous injection in the right flank of NSG mice. After 7 days, the engrafted mice were euthanized, and tumor mass was harvested from the subcutaneous tissue. Tumor tissues were fixed with 4% paraformaldehyde at 4° C. for at least 16 hours. Fixed tissues were then placed in cryomolds, and Tissue-Tek O.C.T. Compound (Sakura, 4583) was carefully added to the cryomolds to embed tumor mass, followed by incubation on dry ice. Tissues were then stored at −80° C., cross-sectioned (5 μm) and preserved on Micro Slides (Corning, 2948-75X25) at −80° C.

Tumor slides were thawed at room temperature. Excess O.C.T. compound was carefully washed with PBS. Tissues were permeabilized with 0.5% Triton X-100 (Sigma-Aldrich, X100) in PBS for 20 minutes at room temperature. Slides were then carefully washed twice with PBS, followed by blocking with 5% BSA (Sigma-Aldrich, A7906-100G) 0.04% sodium azide (Ricca Chemical, 7144.8) in PBS. Primary antibody, rabbit-anti-human IL-1RA polyclonal antibody (Invitrogen, PA5-13428, 1:20) or rabbit-anti-human CD206 monoclonal antibody (clone E2L9N, Cell Signaling Technologies, 91992S, 1:100), was diluted with 5% BSA 0.04% sodium azide in PBS. Tissues were incubated with primary antibody overnight at 4° C. in the dark. On the next day, slides were gently washed with PBS. Goat-anti-rabbit IgG (H+L) cross-adsorbed secondary antibody Cyanine5 (Invitrogen, A10523) was diluted with 5% BSA 0.04% sodium azide in PBS at 1:200. Tissues were then incubated with diluted secondary antibodies for 2-3 hours at room temperature in the dark. Slides were washed gently with PBS, followed by a 10-minute incubation with Hoechst 33342 (Thermo Scientific 62249) diluted in PBS (1:2000) at room temperature in the dark. ProLong Glass Antifade Mountant (Invitrogen, P36980) was then applied onto the tissues before placing cover glass (CardinalHealth, M6045-4A). Excess mountant was eliminated by gently pressing cover glass followed by vacuuming. Slides were then stored at room temperature in the dark overnight till confocal imaging. Images were acquired by ZEISS LSM 980 (ZEISS) at 40X.

### Generation of IL-1RI Knockout CART19-285

A single guide RNA (sgRNA) targeting a sequence in exon 5 of the IL1R1 gene (5′ AAGTCCTCCGTCTCCTGCAA 3′ (SEQ ID NO:9)) was chosen after screening multiple sgRNAs for knockout efficiency. Non-targeting (CT) sgRNA was used to confirm knockout specificity (5′ GCACTTTGTTTGGCCTACTG 3′ (SEQ ID NO:19)). The sgRNAs were cloned into a CAS9 lentiviral construct under the control of a U6 promoter (lentiCRISPRv2, GenScript, Township, NJ, USA). CART19 production with these sgRNA constructs was conducted as described elsewhere (Sterner et al., Journ. Of. Visua. Exper. 149: e59629 (2019)). CTsgRNA CART19 and IL-1R1 KO CART19 were stimulated with JeKo-1 cells at a ratio of 1:1 for 2 days. IL-1R1 expression on CD4⁺ T cells was measured by flow cytometry to confirm knockout efficiency at the protein level.

### Statistical Analysis

Flow cytometry data were analyzed by Kaluza (Beckman Coulter, Chaska, MN, USA). GraphPad Prism (La Jolla, CA, USA) was used for statistical analysis. Adjusted P values on dot plots were identified using the R package ‘clusterProfiler’ of each cell cluster of interest. P values labeled on violin plots were derived from differential gene expression analysis. Figures were generated by Biorender (Toronto, Ontario, Canada), GraphPad Prism, and Kaluza.

### Example 2: Intrinsic Immunosuppressive Features of Monocytes Suppress CART19 Through IL-1 Pathway Modulation in Mantle Cell Lymphoma

The results in this Example re-present and expand on at least some of the results provided in other Examples.

### Results

### Ex Vivo Polarized M2-Like Macrophages Inhibit CART19 Antigen-Specific Proliferation in a Contact-Independent Manner

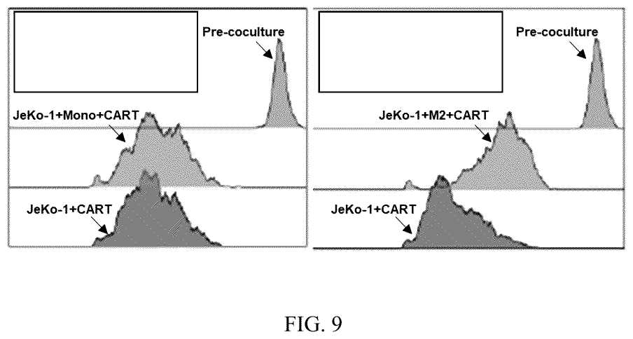

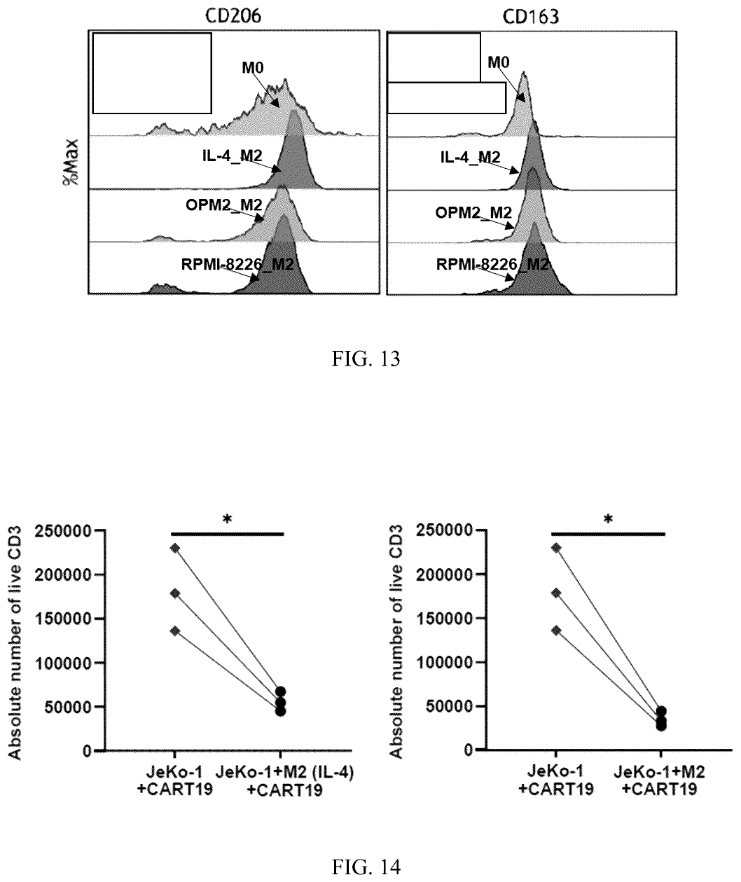

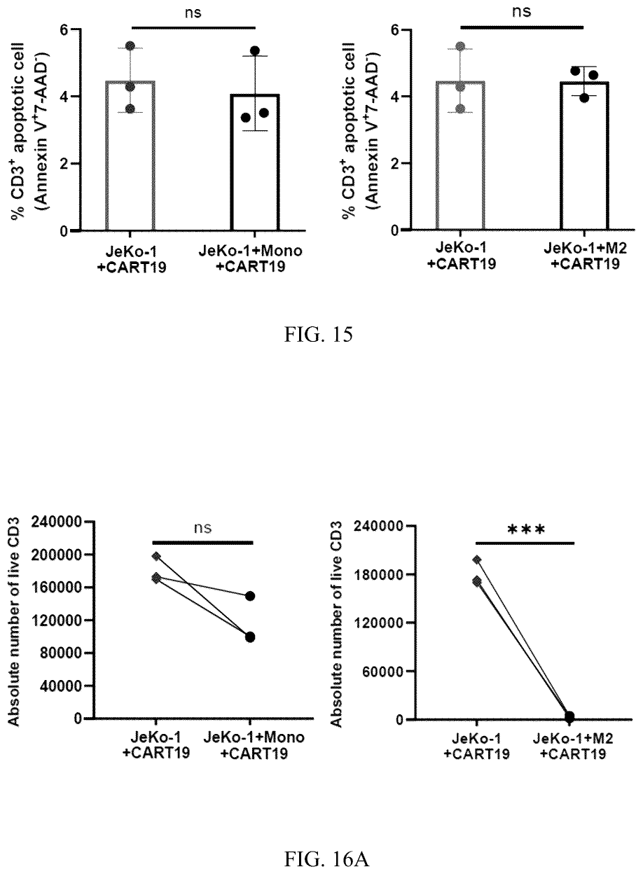

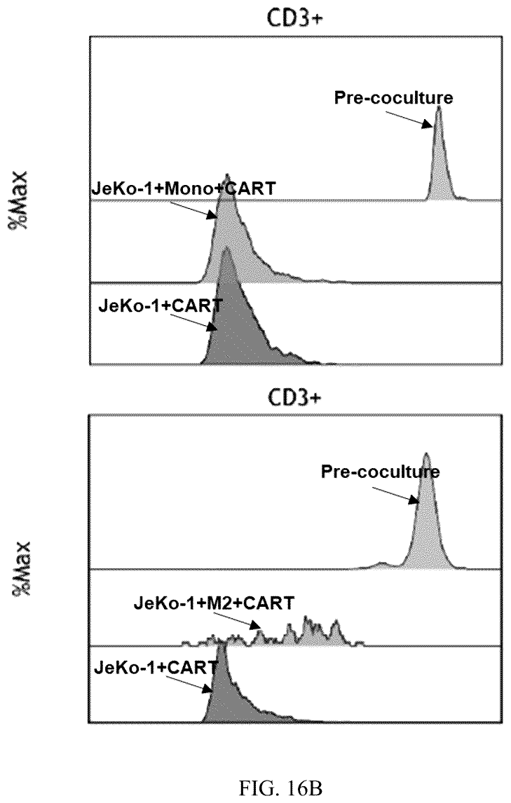

To test the role of IL-1 signaling in immunosuppressive monocyte/macrophage-derived CART inhibition in MCL, the impacts of macrophages on CART19 cell functions were studied in vitro. CD28-costimulated CART19 cells were generated from healthy donors and cocultured with the CD19⁺ MCL cell line JeKo-1 to provide antigen-specific stimulation. Freshly isolated classical monocytes or M2-like macrophages (polarized through coculture with JeKo-1) were added to the cocultures as indicated (FIG. 1A). To generate M2-polarized macrophages, freshly isolated monocytes were cultured in the presence of recombinant human (rh) GM-CSF for 7 days and then cocultured for 24 hours either with medium only as a negative control (M0 macrophage), rh IL-4 as an M2-macrophage positive control, or JeKo-1 cells (FIG. 1B). Expression of CD206 and CD163 was measured on macrophages after ex vivo polarization, confirming M2-like phenotypes on macrophages cultured with either rh IL-4 or JeKo-1 (FIG. 1C). Polarization of monocytes into M2-like macrophages was seen with other cancer cell lines in addition to JeKo-1 (FIG. 13). Next, cocultures of CART19, JeKo-1, and fresh monocytes or ex vivo JeKo-1-polarized M2-like macrophages were conducted at a ratio of 2:2:1 for 3 days. CART19 antigen-specific proliferation was inhibited in the presence of M2-like macrophages but not fresh monocytes (FIG. 1D-1E). To further validate the immunosuppressive functions of M2-like macrophages polarized by JeKo-1, CART19, JeKo-1, and rh IL-4-differentiated M2-like macrophages were cocultured for 3 days. Similar inhibition capacity was seen between IL-4- and JeKo-1-polarized M2-like macrophages (FIG. 14). CART cell division as determined by CFSE intensity changes after the 3-day cocultures was significantly reduced in M2-like macrophage cocultures (FIG. 9). To understand how M2-like macrophages impact the rate of CART cell apoptosis, an Annexin V/7-AAD apoptosis assay was performed in these cocultures, which showed that M2-like macrophages did not affect CART cell apoptosis (FIG. 15), implying that M2-like macrophages inhibit CART antigen-specific expansion mainly through suppressing cell division. These cocultures were repeated over 7 days and showed consistent M2-like macrophage-mediated CART proliferation inhibition indicated by CART antigen-specific expansion and CART cell division by CFSE staining (FIG. 16A-16B).

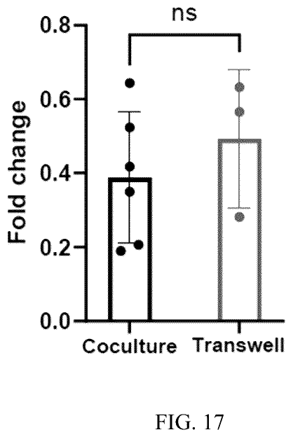

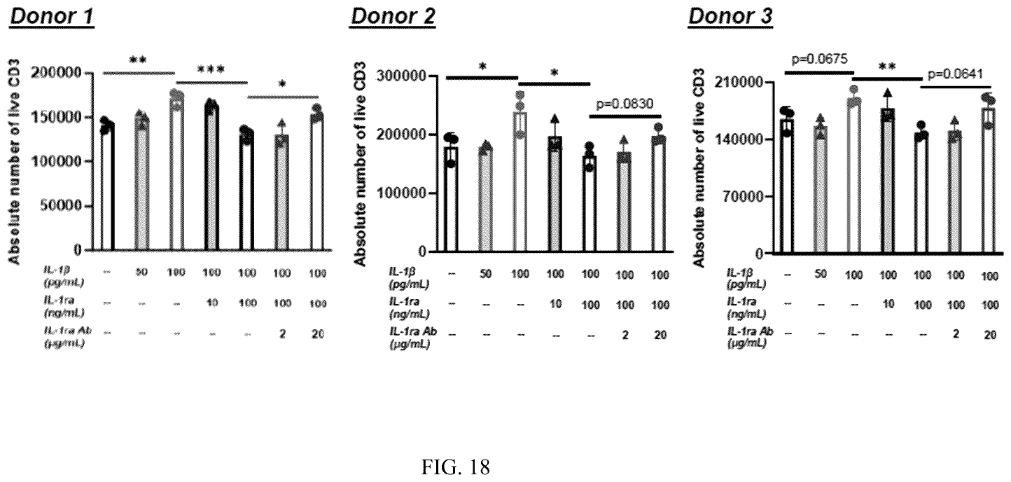

The mechanisms by which M2-polarized macrophages inhibit CART19 cells in MCL was evaluated. Studies were focused on the expression of inhibitory receptors/ligands on activated macrophages as well as the secretion of inhibitory cytokines. Cells from the cocultures (described in FIG. 1A) were harvested and assessed for expression of TRAIL-R2, Fas, PD-1, PD-L1 on T cells and TRAIL, FasL, PD-L1, PD-L2 on macrophages. There were no significant differences in expression of these inhibitory markers between fresh monocyte and M2-like macrophage groups across multiple biological replicates (FIG. 7A-7D). This suggested that M2-like macrophage-derived immunosuppression of CART19 might be primarily mediated by soluble factors rather than inhibitory receptor/ligand interactions in this preclinical model. To assess if this inhibition requires direct cell-cell contact, a transwell assay was conducted where M2-like macrophages and JeKo-1-activated CART19 were physically separated while soluble molecules were allowed to traffic freely. CART19 proliferation was significantly inhibited in the presence of M2-like macrophages (FIG. 1F), with suppression similar in magnitude to direct CART19-macrophage-tumor cocultures (FIG. 17), confirming contact-independent mechanisms of suppression. Cytokine analyses were performed on the supernatants from these cocultures (FIG. 1G). To characterize the cytokine profiles of stimulated macrophages without additional cytokines secreted from activated CART19, supernatants from cocultures of JeKo-1, monocytes, and untransduced T cells (UTD) were compared to cocultures of JeKo-1, M2-like macrophages, and UTD. IL-1ra was significantly elevated in the presence of M2-like macrophages in all donors (FIG. 1H). Comparing the cocultures containing CART19, significantly increased secretion of IL-1ra and reduced secretion of multiple chemokines and IL-1β production-associated cytokines, including GRO, MCP-3, IL-1β, MIP-1α, MIP-1β, sCD40L, and IL-17A were observed when M2-like macrophages were present (FIG. 1I). This suggested that M2-like macrophages inhibit CART19 in MCL at least partially through downregulating IL-1 signaling mediated by secretion of IL-1ra.

### Neutralizing IL-Ira Improved CART19 Effector Functions Both In Vitro and In Vivo

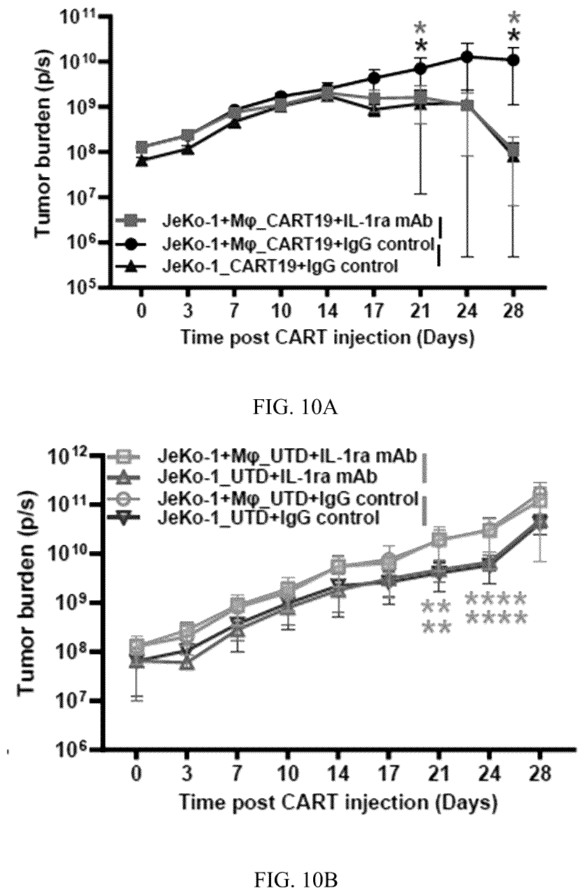

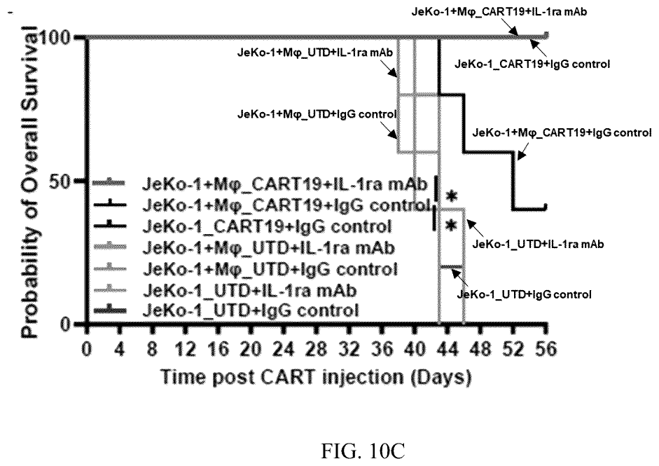

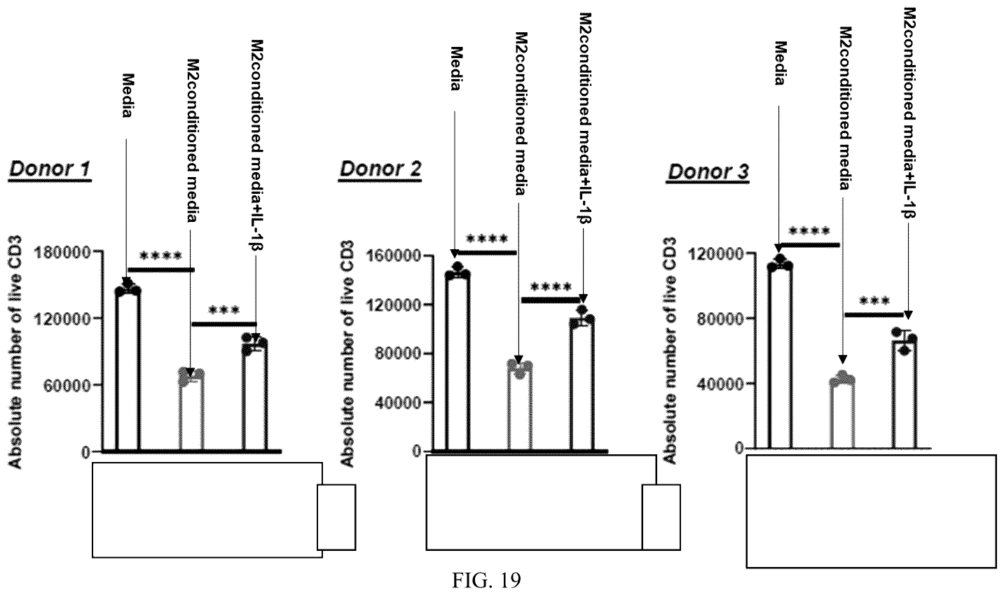

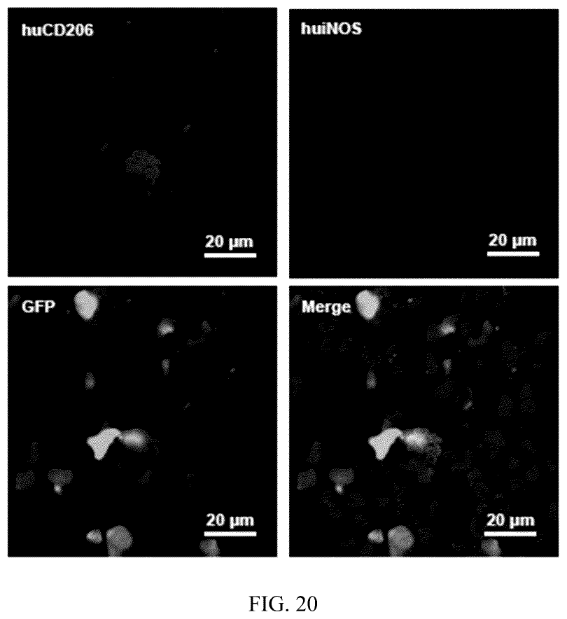

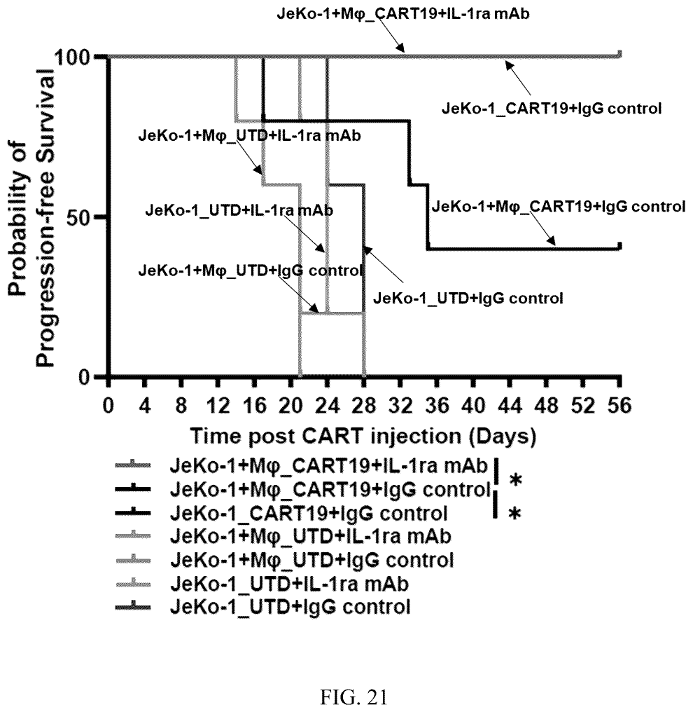

M2-like macrophages have been found to be the predominant producer of IL-1ra. To study the effect of IL-1ra on CART19 function directly, CART19 with JeKo-1 were stimulated and the media was supplemented with recombinant human IL-1β alone, IL-1β combined with IL-1ra, or vehicle control for 3 days. Doses were based on the manufacturer's recommendations and further confirmed by a dose titration experiment (FIG. 18). The T cell proliferation assay demonstrated that IL-1β enhanced CART19 antigen-specific proliferation, which was then inhibited by IL-1ra and by M2-conditioned media (FIG. 5A and FIG. 19), implying that IL-1ra suppressed IL-1β-dependent CART19 proliferation. In the same experiment, IL-1ra was neutralized by adding IL-1ra neutralizing antibody and showed that blocking IL-1ra preserved IL-1β-enhanced CART proliferation (FIG. 5A). Next, the impact of M2-like macrophage-derived IL-1ra was tested in a mantle cell lymphoma xenograft mouse model engrafted with both tumor and macrophages. Here, M0 macrophages and luciferase⁺ JeKo-1 cells resuspended in MATRIGEL® at a ratio of 1:2 were subcutaneously engrafted in NOD-SCID-γ^{-/-} (NSG) mice. Seven days later, tumor tissues were dissected and prepared for immunofluorescence imaging for macrophage phenotyping. The engrafted M0 macrophages developed M2-like phenotypes (human CD206⁺) and expressed human IL-1ra with undetectable M1-like phenotype indicated by expression of human iNOS (FIG. 5B and FIG. 20). After validating in vivo M2-like macrophage polarization, the therapeutic effects of IL-1ra neutralizing antibody on CART19 efficacy were assessed in this model. NSG mice were subcutaneously engrafted with either 1×10⁶ luciferase⁺ JeKo-1 plus 5×10⁵ M0 macrophages or 1×10⁶ luciferase⁺ JeKo-1 alone in MATRIGEL®. When tumor engraftment was confirmed with bioluminescence imaging (BLI), mice were randomized to treatment with an intravenous injection of CART19 in combination with 10 mg/kg IL-1ra neutralizing antibody or control IgG (intraperitoneally twice weekly for three weeks), UTD in combination with control IgG, or left untreated. Tumor burden was assessed by serial BLI (FIG. 2C). Reduced tumor burden was observed in mice engrafted with macrophages and treated with CART19 in combination with IL-1ra neutralizing antibody compared to mice engrafted with macrophages and treated with control IgG (FIG. 10A). Antitumor activity of CART19 in the presence of macrophages and IL-1ra blockade was similar to the antitumor activity of CART19 with control IgG in the absence of macrophages (FIG. 10A). This indicates that IL-1ra antibody treatment was able to mitigate the detrimental effect of macrophages on CART19 antitumor activity. In the IgG control-treated groups, mice engrafted with macrophages exhibited faster tumor growth than mice engrafted with JeKo-1 alone (FIG. 10A), further demonstrating that M2-like macrophages promoted tumor growth. In addition, neutralizing IL-1ra did not have significant impact on tumor growth when mice were treated with control UTD, indicating IL-1ra neutralization specifically acted on tumor-activated CART cells (FIG. 10B). Overall survival was improved in mice treated by CART19 in combination with IL-1ra neutralizing antibody in the presence of macrophages (FIG. 10C and FIG. 21).

IL1-ra inhibition of IL-Iβ mediated CART19 proliferation is dependent on interactions with IL-1RI on stimulated CART19

Finally, how M2-like macrophages interact with the IL-1 receptors on CART19 cells was analyzed. The expression of IL-1 receptor I (IL-1RI), the cognate receptor of both IL-1ra and IL-1β, on T cells was evaluated. CART19 and JeKo-1 were cocultured at a 1:1 ratio for 3 days, during which IL-1RI expression was analyzed on both CD4 and CD8 T cells daily. IL-1RI expression was significantly enhanced in stimulated T cells, predominantly found on CD4 T cells, and generally peaked 24 hours after initiation of coculture (FIG. 6A). Next, M2-like macrophages were incorporated into the cocultures (FIG. 1A). It was found that the presence of M2-like macrophages induced downregulation of IL-1RI on JeKo-1-stimulated CART cells (FIG. 6B), which might be due to the binding of IL-1ra highly secreted by M2-like macrophages to IL-1RI on stimulated T cells. To determine if IL1-ra inhibits CART19 cells by binding to IL-1RI, we generated IL-1RI knockout CART19 with CRISPR/Cas9 by lentiviral transduction. Knockout efficiency was confirmed on the protein level by flow cytometry (FIG. 6C and FIG. 8). To study response of IL-1RI^{k/o} CART19 to IL-1, engineered CART19 and JeKo-1 supplemented with rhIL-1β were cocultured with or without IL-1ra for 3 days. No significant response, as indicated by CART antigen-specific proliferation, to IL-1 stimulation was observed (FIG. 6D). These data indicate that both IL-1β and IL-1ra effects are mediated by their interactions with IL-1RI on stimulated CART19 cells.

Collectively, these studies suggest that immunosuppressive M2-like macrophages inhibit CART19 in a contact-independent manner, at least partially by impairing IL-1β signaling in CART cells through IL-1ra secretion.

Downregulated IL-1β Signaling and its Downstream Pathways were Detected in Baseline CART19 Subset Populations from Non-Responders in the Phase 2 ZUMA-2 Trial

To validate these findings, clinical samples from the ZUMA-2 trial were analyzed. Single cell RNA sequencing (scRNAseq) analysis was performed on cryopreserved baseline autologous brexu-cel preinfusion products from patients with MCL treated with brexu-cel in the ZUMA-2 clinical trial. In this clinical trial, 4 patients did not respond (stable or progressive disease), and 3 patients subsequently relapsed¹⁰. ScRNAseq was performed on baseline products from these 7 patients as well as from 5 matched responders who achieved complete remission (Table 3). In total, 90,177 cells were sequenced from responder, relapsed, and non-responder samples and clustered (FIG. 2A). All downstream RNA expression analyses were conducted based on comparisons between non-responders (42,836 cells) versus initial responders (relapsed and responders, 47,341 cells) within each T cell population of interest (FIG. 2A). We first analyzed and compared proportion of each T cell population between initial responders and non-responders brexu-cel products (FIG. 11). No significant differences were detected between initial responders and non-responders (FDR>0.05), indicating that frequency of CART cells with certain phenotypes in the infusion product did not contribute to treatment failure in the ZUMA-2 trial.

TABLE 3
Selected Demographics and Baseline Characteristics (Overall:
KTE-X19) (Safety Analysis Set: 12 Selected Patients)

| | Responders | | Relapsed | | Non-responders | |
|---|---|---|---|---|---|---|
| | (N = 5) | | (N = 3) | | (N = 4) | |
| Age (years) | | | | | | |
| | 5 | | 3 | | 4 | |
| Mean (StD) | 62.0 | (6.3) | 62.7 | (7.6) | 69.0 | (5.2) |
| Median | 61.0 | | 61.0 | | 69.0 | |
| Min, max | 55, 69 | | 56, 71 | | 64, 74 | |
| Age Category, n (%) | | | | | | |
| <65 Years | 3 | (60) | 2 | (67) | 1 | (25) |
| ≥65 Years | 2 | (40) | 1 | (33) | 3 | (75) |
| Sex, n (%) | | | | | | |
| Male | 3 | (60) | 3 | (100) | 3 | (75) |
| Female | 2 | (40) | 0 | | 1 | (25) |
| Disease stage, n (%) | | | | | | |
| I | 0 | | 0 | | 0 | |
| II | 0 | | 1 | (33) | 0 | |
| III | 0 | | 0 | | 0 | |
| IV | 5 | (100) | 2 | (67) | 4 | (100) |
| [symbol] | | | | | | |
| Low risk | 2 | (40) | 1 | (33) | 0 | |
| Intermediate risk | 3 | (60) | 1 | (33) | 1 | (25) |
| High risk | 0 | | 1 | (33) | 2 | (50) |
| Missing | 0 | | 0 | | 1 | (25) |
| LDH relative to upper limit, n (%) | | | | | | |
| LDH < 0.67 ULN | 1 | (20) | 0 | | 0 | |
| 0.67 ULN ≤ LDH < ULN | 1 | (20) | 2 | (67) | 2 | (50) |
| ULN ≤ LDH < [symbol] | 3 | (60) | 0 | | 1 | (25) |
| 1.5 ULN ≤ LDH | 0 | | 1 | (33) | 0 | |
| Missing | 0 | | 0 | | 1 | (25) |
| Number of prior regimens, n (%) | | | | | | |
| n | 5 | | 3 | | 4 | |
| Mean (StD) | 3.4 | (0.9) | 3.7 | (1.2) | 3.0 | (1.6) |

| | | | |
|---|---|---|---|
| Median | 3.0 | 3.0 | 3.0 |
| Min, max | 3, 5 | 3, 5 | 1, 5 |

Data cutoff date = 5 Oct. 2023
Abbreviations: LDH, lactose dehydrogenase; StD, standard deviation; ULN, upper limit of normal.
Note:
Percentages are based on the total number of subjects in each column.
Data Source: ADSL, [symbol]
Program Name: [symbol]
Output Generated: [symbol] [symbol]
indicates data missing or illegible when filed

Pathway analysis demonstrated downregulation of T cell function associated pathways in CART populations of non-responders (FIG. 2B). Subsequently, IL-1 response associated pathways were evaluated in CART populations. Specifically, CD4 CART19 cells from non-responders showed reduced T-helper (Th) 17 type immune responses and differentiation (FIG. 2B), implying impaired IL-1β signaling in non-responders given that IL-1β signaling is essential for Th17 differentiation²⁵. Response to IL-1 was found to be downregulated in activated CD8 CART19 from the non-responders as well (FIG. 2B). These data implied that downregulated IL-1 signaling in brexu-cel products might have contributed to CART resistance development in the non-responders of the ZUMA-2 trial.

M2-Like Phenotypes and Downregulation of IL-1β Production Pathways were Detected in Peripheral Monocyte Populations from Non-Responder Treated in the Phase 2 ZUMA-2 Clinical Trial

Next, it was evaluated whether this observed downregulation of IL-1 signaling in brexu-cel products is associated with a specific myeloid cell signature at baseline in the non-responders. cryopreserved autologous baseline PBMC samples, which were collected prior to lymphodepletion chemotherapy and CART infusion, from the same cohort of patients with MCL treated with brexu-cel in the ZUMA-2 clinical trial and whose brexu-cel products were sequenced (as shown in FIG. 4) were interrogated. Cryopreserved baseline PBMCs (prior to brexu-cel treatment) from these 12 patients were recovered and prepared for scRNAseq analysis. Samples from 3 responders, 3 relapsed, and 1 non-responder were qualified for downstream RNA analysis according to quality control screening. Monocyte/macrophage populations, including M2-like monocytes, M1-like monocytes and classical monocytes, were identified in patient PBMCs based on scRNAseq data (FIG. 3A). In total, 41,002 cells were sequenced from responder, relapsed, and non-responder samples. All downstream RNA expression analyses were conducted based on comparisons between non-responder (871 cells) versus initial responders (40,131 cells) within each cell population of interest.

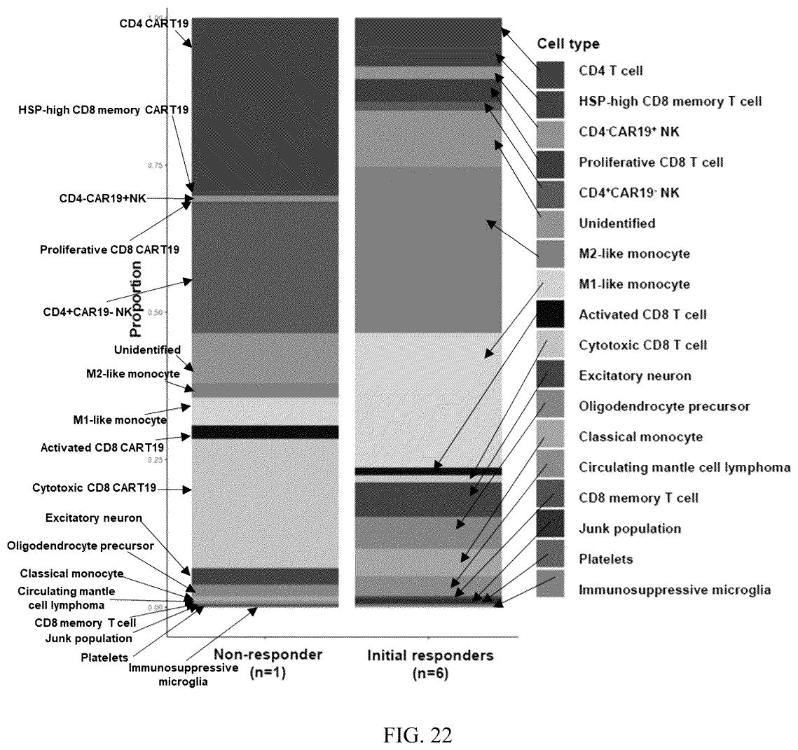

Cell proportion analysis indicated no significant difference in the frequency of classical monocyte, M1-, and M2-like monocyte between initial responders and non-responder was observed (FDR>0.05, FIG. 22), suggesting that frequency of the circulating myeloid cells did not play an essential role in CART failure of ZUMA-2 trial. Differential gene expression analysis revealed significant enrichment of HMOX1, a gene which is expressed in macrophages and negatively regulates IL-1β production, and downregulation of EGR1, whose expression is induced by IL-1β, in M2-like monocytes of the non-responder compared to initial responders (FIG. 3B). Pathway analysis demonstrated downregulation of IL-1β production as well as T cell mediated immunity in M2-like monocytes within the non-responder (FIG. 3C).

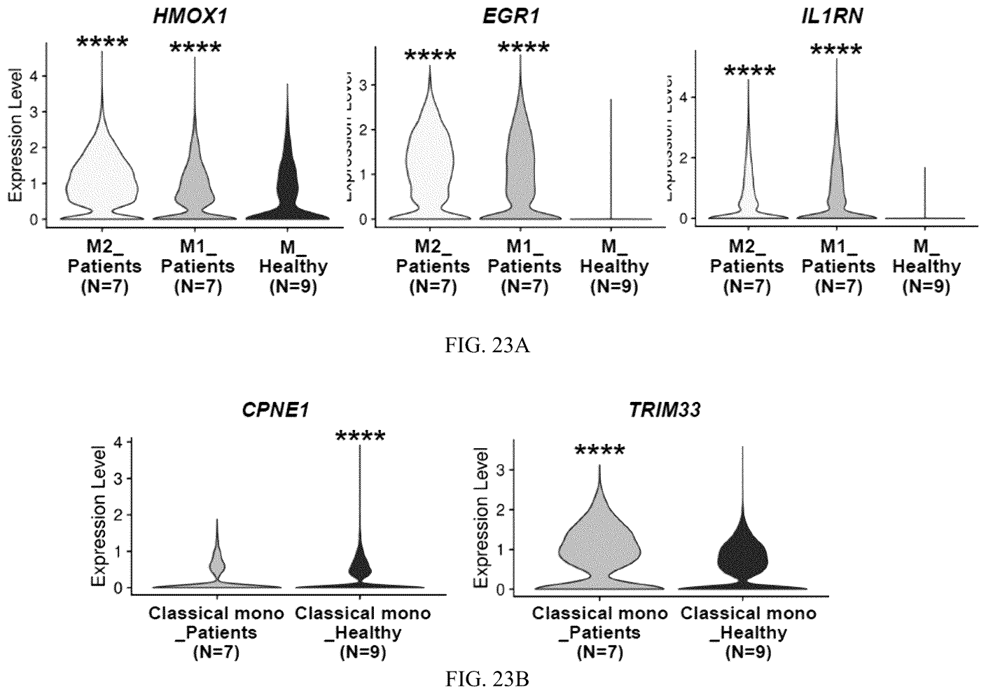

Next, differentially expressed genes and pathways of the M1-like monocyte population were analyzed. Enrichment of IL1RN, the gene that encodes IL-1ra, and downregulation of EGR1 in M1-like monocytes from the non-responder were detected (FIG. 3B). HMOX1, EGR1, and IL1RN were significantly enriched in monocytes from patients with MCL compared to monocytes from healthy individuals, based on scRNAseq analyses of publically available datasets (FIG. 23A). The corresponding pathway analysis indicated decreased IL-1β production and reduced positive regulation on T cell functions as well as cytokine/chemokine production in the non-responder M1-like monocytes (FIG. 3C), which was consistent with our observations in the M2-like monocyte population.

Classical monocyte differential gene expression and signaling pathways analyses were performed. Upregulation of CPNE1, overexpression of which leads to higher expression of CD206 and CD163, and TRIM33, a gene essential to M2 polarization, were detected in the non-responder (FIG. 3B). TRIM33 was significantly more enriched in classical monocytes from patients with MCL compared to its level in classical monocytes from healthy individuals (FIG. 23B). These data suggest that classical monocytes of the non-responder had the tendency to differentiate and acquire immunosuppressive phenotypes inhibiting T cell immunity.

### Comparative Analysis of IL-1-Associated Cytokines Based on Differential Gene Expression Analysis

Finally, violin plots were generated on mRNA expression of ILIA, IL1B, IL1RN, IL1R1, IL1R2, IL1RAP, IL6, IL6R, IL10, IL10RA, and IL10RB in each monocyte population of interest (FIG. 3E-3G). Trends of elevated expression of IL1B and reduced expression of IL1RN in initial responders were observed in all three cell populations of interest (FIG. 3E-3G), which was consistent with our pathway analysis. IL1R1 expression showed increasing trends while IL1R2 and IL1RAP showed downregulation in all clusters of interest in the non-responder (FIG. 3E-3G). IL6 and IL6R expression were found to be elevated in initial responders compared to the non-responder (FIG. 3E-3G). IL10 expression was enriched in the non-responder within M2-like monocyte and classical monocyte populations (FIGS. 3E and 3G).

Altogether, the single cell sequencing analyses of peripheral monocytes from the non-responder showed upregulation of genes involved in M2-like differentiation and IL1RN as well as downregulation of IL-1β production and T cell mediated immunity pathways.

Collectively, the findings from the ZUMA-2 clinical samples implied impaired IL-1 signaling in the brexu-cel products of the non-responders in association with IL-1 production in non-responder monocytes, which is consistent with our preclinical findings.

IL-Iβ induced higher CART cell division and apoptosis in IL-1RI⁺ T cells and enhanced CART in vivo antitumor activities

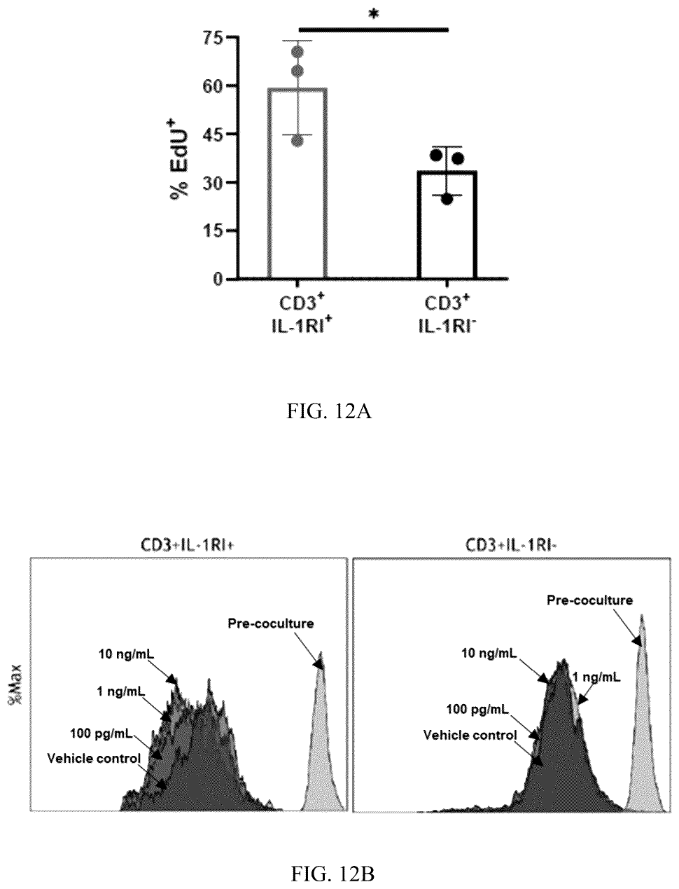

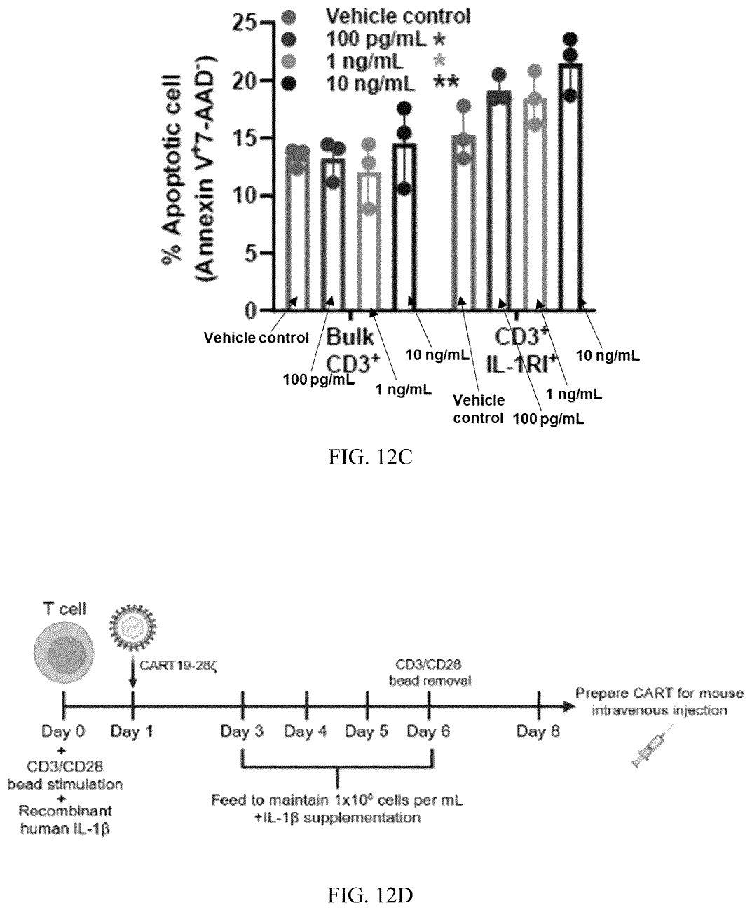

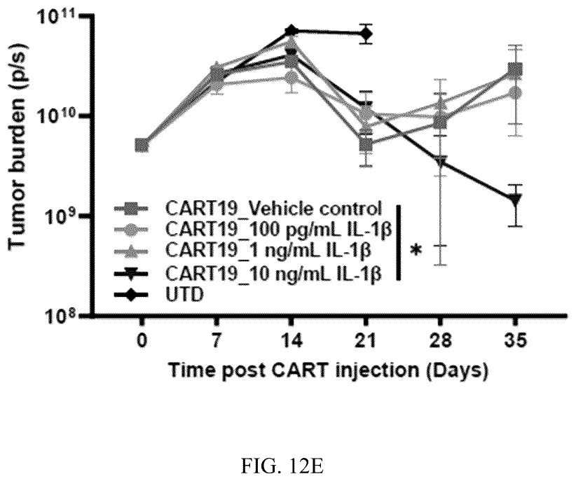

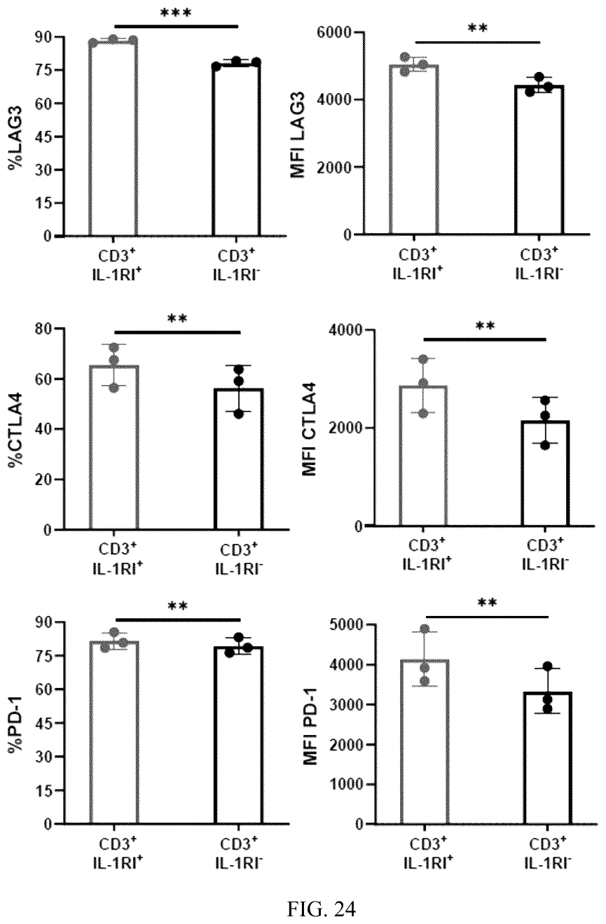

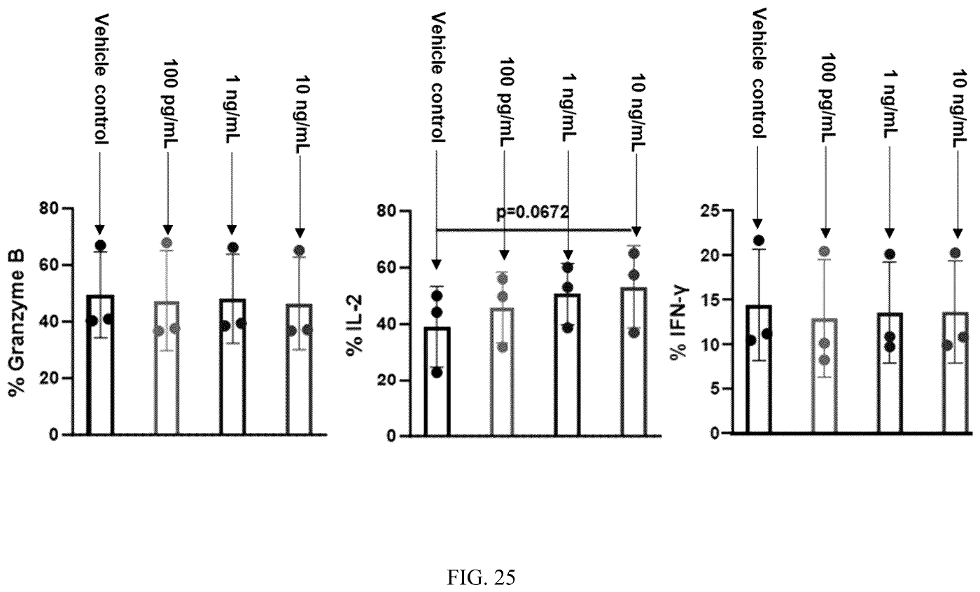

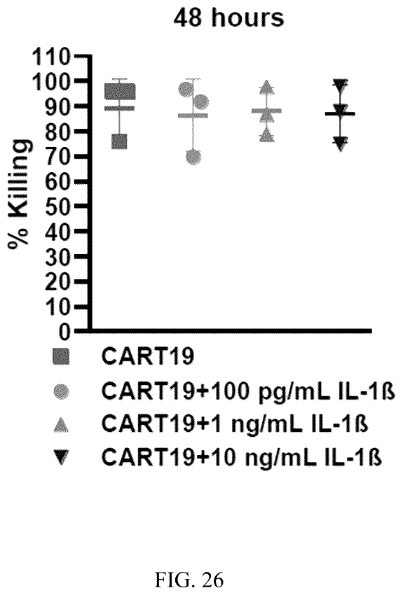

Given the observed contribution of IL-1β signaling to CART cell activities in the study, T cells expressing its cognate receptor IL-1RI were further characterized to understand functional differences from IL-1RI⁻ T cells. EdU assay suggested higher frequency of proliferative T cells in IL-1RI⁺ T cells than in the IL-1RI⁻ T cell subset (FIG. 12A). Moderately higher expression of T cell inhibitory markers PD-1, LAG3, and CTLA4 were observed on IL-1RI⁺ T cells (FIG. 24). Next, it was investigated how IL-1β impacts CART cell functions and phenotypes. CFSE-stained CART cells were cocultured with JeKo-1 for 3 days supplemented with different doses of IL-1β. Moderate reduction of CFSE was observed in IL-1RI⁺ T cells with the addition of IL-1β, implying that IL-1β promoted T cell proliferation through expression of IL-1RI (FIG. 12B). An annexin V/7-AAD apoptosis assay indicated a higher frequency of apoptotic cells in IL-1RI⁺ T cell subsets from cocultures with IL-1β (FIG. 12C). Additionally, expression of effector cytokines, including granzyme B, IL-2, and IFN-γ, were intracellularly measured in CART cells by flow cytometry after a 4-hour coculture with JeKo-1 and IL-1β supplementation. No significant difference was detected in these cytokines between vehicle control and IL-1β groups (FIG. 25). Tumor cytotoxicity of CART cells was also not significantly impacted by IL-1β (FIG. 26). Next, we studied how IL-1β impacts CART in vivo antitumor activities. Briefly, different doses of IL-1β were added during the 8-day CART production (FIG. 12D). NSG mice were injected with 1×10⁶ luciferase⁺ JeKo-1 intravenously. When tumor engraftment was confirmed with high BLI for a stress model, mice were randomized to treatment with an intravenous injection of 1×10⁶ CART or IL-1β-pre-exposed CART cells. Delayed tumor progression was detected in mice treated with CART pre-exposed to 10 ng/ml IL-1β compared to CART without IL-1β addition (FIG. 12E). Collectively, these data suggested that IL-1β-stimulated and IL-1RI⁺ T cells showed more proliferative capacity and more apoptotic phenotype, indicating increased activation and functionalities.

### Materials & Methods

### Cell Lines

All cell lines were maintained at 37° C. and 5% CO₂. JeKo-1, a CD19⁺ mantle cell lymphoma cell line, was purchased from ATCC (CRL-3006, Manassas, VA, USA). JeKo-1 luciferase⁺ cell line was generated by transduction with luciferase-ZsGreen lentivirus (Addgene, Cambridge, MA, USA) and sorted to 100% purity. Cell lines were maintained in R20 medium composed of RPMI 1640 (Gibco, 11875093, Gaithersburg, MD, USA), 20% FBS (Corning Life Sciences, 35-011-CV, Corning, NY, USA), and 1% penicillin-streptomycin-glutamine (Gibco, 10-378-016).

### Monocyte Isolation and Ex Vivo M2-Like Macrophage Polarization

PBMCs were isolated from de-identified healthy donor apheresis cones by density gradient centrifugation and resuspended in 2% FBS in PBS (Gibco). Human classical monocytes were isolated from fresh PBMCs by classical monocyte isolation kit (Miltenyi Biotec, 130-117-337, Bergisch Gladback, Germany) according to the manufacturer's instructions. All in vitro and ex vivo cultures were maintained in T cell medium (TCM) containing X-VIVO 15 (Lonza, 02-060Q, Walkersville, MD, USA), 10% heat-inactivated human AB serum plasma (Innovative Research, Inc., 44277, Novi, MI, USA), and 1% penicillin-streptomycin-glutamine. Purity (>90% CD14⁺ CD16⁻) was confirmed by flow cytometry. To differentiate classical monocytes into M0 macrophages, freshly isolated monocytes were resuspended in TCM at a concentration of 1×10⁶/mL and incubated with 10 ng/mL rhGM-CSF (STEMCELL, 78015, Vancouver, Canada) at 37° C. for 7 days. To generate M2-like macrophages, M0 macrophages were incubated in TCM with either 20 ng/mL rhIL-4 (STEMCELL, 78045) or JeKo-1 cells at a ratio of 1:2 for another 24 hours. Differentiated macrophages were detached from tissue culture plates through incubation on ice for at least 30 minutes. Cells were detached by pipetting every 10 minutes with ice cold PBS. Adherence of residual cells was checked under a microscope until all macrophages were detached. M2 phenotype was confirmed by flow cytometry (CD14⁺ CD163^{high} CD206^{high}). M0 macrophages were used as a baseline control to distinguish M2-like phenotypes in differentiated macrophages.

### In Vitro Coculture of CART19 and Monocytes or Macrophages

CART19-28ζ was generated from healthy donor PBMCs as described herein. Freshly isolated monocytes or M2-like macrophages were cocultured with CART19 and JeKo-1 cells at a ratio of 1:2:2 at 37° C. Cells were collected on Day 3 and assessed for CART19 antigen-specific proliferation (absolute counts of CD3⁺ cells as previously described⁴³) as well as expression of surface markers of interest on both T cells (CD3, PD-1, PD-L1, Fas, and TRAIL-R2) and monocytes (CD14, TRAIL, FasL, PD-L1, and PD-L2). Supernatants of cocultures were also collected and cryopreserved at −80° C. for cytokine analysis. Cytokines of interest were quantified by MILLIPLEX MAP Human Cytokine/Chemokine Magnetic Bead Panel (Millipore Sigma, HCYTMAG-60K-PX38, Ontario, Canada) according to the manufacturer's instructions.

For transwell assays, M2-like macrophages were generated (Ruella et al., Cancer Discov., 7:1154-1167 (2017)) and seeded in the bottom chamber of a transwell plate (0.4 μm). JeKo-1 and CART19 cells were added at a ratio of 1:1 into the upper wells on Day 8 of the M2-like macrophage differentiation timeline. Cells were cocultured at 37° C. for an additional 3 days. On Day 11, cells in the upper chambers were harvested and washed with PBS for subsequent analysis.

For rhIL-4-differentiated M2-like macrophage coculture with CART cells, M0 macrophages were incubated in TCM with 20 ng/mL rhIL-4 (STEMCELL, 78045) for another 24 hours. CART and JeKo-1 were added to the coculture at a ratio of 2:2:1 for 3 days. On Day 3, cells were harvested and processed for CART proliferation assay.

### Cfse Staining of CART Cells Prior to Cocultures

CFSE was reconstituted according to manufacturer's instructions (C34554, ThermoFisher). CFSE was further diluted with pre-warmed PBS to 0.1 μM. CART cells were washed with PBS and resuspended in diluted CFSE to reach 1×10⁶/mL. Cells were incubated in the dark at room temperature for 8 minutes. CART cells were washed at least twice with T cell media. CSFE intensity in CART cells prior to cocultures was measured by flow cytometry. CSFE stained CART cells were cocultured with JeKo-1 and monocytes or M2-like macrophages or different doses of rhIL-1β for 3 days. CFSE intensity in CART was measured by flow cytometry on day 3.

### Apoptosis Assay of CART by 7-AAD and Annexin V

CART cells were cocultured with JeKo-1 and monocytes or M2-like macrophages or different doses of rhIL-1β for 3 days. Expression of 7-AAD and Annexin V was measured in CART cells on day 3. Apoptotic cells were defined as Annexin V⁺ 7-AAD⁻.

### Edu Assay

CART cells were resuspended in TCM at 1×10⁶/mL with addition of EdU to reach a final concentration of 2 μM (C10634, ThermoFisher). CART cells were then cocultured with JeKo-1 at a ratio of 1:1 for 24 hours. Cells were washed 1% BSA-PBS and fixed with 4% PFA for 15 minutes at room temperature. Cells were permeabilized with Click-iT permeabilization/wash solution for 15 minutes at room temperature. EdU detection was performed following manufacturer's instructions.

### Single-Cell RNA Sequencing

### Sample Preparation

Cryopreserved patient PBMCs and autologous brexu-cel products from 5 responders, 3 relapsed, and 4 non-responders from the ZUMA-2 clinical trial were carefully thawed and washed once with TCM. Cells were resuspended in 0.04% human AB serum in PBS at a concentration of >1×10⁵ cells/mL. Prepared samples were subjected to quality control screening, library preparation, single-cell RNA sequencing, and downstream analysis. Due to low cell count/poor cell quality, PBMCs from 3 responders, 3 relapsed, and 1 non-responder were qualified for downstream RNA sequencing analysis. Brexu-cel samples from all patients were processed for downstream RNA sequencing analysis.

### Mapping

The single cell sequence preprocessing was performed using the standard 10× Genomics Cell Ranger Single Cell Software Suite. Raw reads were aligned to the hg38 reference genome. UMI (unique molecular identifier) counting was performed using Cell Ranger v7.1.0 pipeline with default parameters.

### Quality Control, Integration, Clustering, and Differential Expression Analysis

Seurat_4.9.9 was used for all following analyses. All genes that were not detected in at least three single cells were excluded. Cells in which fewer than 200 unique genes were detected were excluded to remove possible low-quality cells. Mitochondrial quality control metrics were calculated using the ‘PercentageFeatureSet’ function to filter out cells with >40% mitochondrial counts to exclude low-quality and dying cells. Normalization was performed by the global-scaling normalization method ‘LogNormalize’ in Seurat, and then the results were log-transformed for downstream analysis. Then the ‘FindVariableFeatures’ function was used to calculate a subset of highly variable features (default: 3000 genes) for each sample to highlight biological signals in future analyses. A linear transformation as a standard pre-processing step was performed on the combined dataset. Then, Harmony R package was applied to integration analysis for batch effect removal and primary clustering. Principal component analysis was conducted to determine the dimensionality of the dataset, and UMAP was used to visualize the combined dataset. The ‘FindNeighbors’ and ‘FindClusters’ functions were applied to cluster cells, cluster-specific markers conserved across conditions were identified with the ‘FindConservedMarkers’ function, and clusters were assigned to known cell types based on their specific markers. After all the cells were aligned, comparative analysis was performed to identify DEGs induced by different conditions. Genes with P value <0.05 and |log₂fold change|>1 were defined as significant DEGs. All marker genes and DEGs were visualized with ‘FeaturePlot’ and ‘VlnPlot’ functions in Seurat and the R package ‘ggplot2’ (https://ggplot2.tidyverse.org). R package ‘clusterProfiler’ was applied for DEG set enrichment analysis and visualization. Only functional categories related to IL-1 pathway and T cell immunity were chosen.

### In Vitro Functional Assay of IL-1β and IL-1Ra

CART19 cells resuspended at 1×10⁶ cells/mL were cocultured with JeKo-1 at a ratio of 1:1 for 3 days. 100 μg/mL rhIL-1β (R&D systems, 201-LB-010), 100 ng/mL rhIL-1ra (R&D systems, 280-RA-010), or 20 μg/mL IL-1ra neutralizing monoclonal antibody (clone 10309, Invitrogen, MA5-23802) were added at the beginning of the coculture according to the indicated experimental conditions. On Day 3, cells were harvested and processed for CART proliferation assay.

Cart Antigen-Specific Expansion with M2-Like Macrophage Conditioned Media

M2-like macrophages were differentiated by JeKo-1 cells from classical monocytes as described herein. Supernatants were collected from M2-like cocultures. CART cells were cocultured with cell culture media, M2-like macrophage conditioned media, or M2 conditioned media with addition of 100 pg/mL rhIL-1β (R&D systems, 201-LB-010) for 3 days. On Day 3, cells were harvested and processed for CART proliferation assay.

### Intracellular Staining of Cytokines in CART Cells

CART cells were cocultured with JeKo-1 at a ratio of 1:1 for 4 hours with addition of different doses of rhIL-1β. Cells were fixed with Fixation Medium A (GAS001S5, ThermoFisher) followed by permeabilization with Permeabilization Medium B (GAS002S5, ThermoFisher). Expression of IL-2, granzyme B, and IFN-γ were measured by flow cytometry.

### Tumor Cytotoxicity Assay

CART wells were cocultured with luciferase⁺ JeKo-1 at a ratio of 1:1 for 48 hours in 96-well black plates. Bioluminescence intensity (BLI) was measured at 48 hours. 3 μg D-luciferin (15 mg/mL, Gold Biotechnology, St. Louis, M0, USA) was added per 200 μL. Plates were read on Promega plate reader for BLI.

### Mantle Cell Lymphoma Xenograft Subcutaneous Mouse Model

Male and female 6-8 weeks old NOD-SCID-γ^{-/-} (NSG) mice were housed in an animal barrier space.

Human M0 macrophages were differentiated in vitro as previously described. Cells were carefully detached and harvested on ice and washed by cold PBS, prior to resuspension in PBS to reach a final concentration of 1×10⁷ cells/mL. Luciferase⁺ JeKo-1 cells were washed with cold PBS and resuspended to reach 2×10⁷ cells/mL. Macrophages and luciferase⁺ JeKo-1 were thoroughly mixed at a 1:2 ratio. For mice receiving JeKo-1 alone, tumor cells were mixed with same volume of PBS instead. 100 μL of prepared cells were mixed with 100 μL of MATRIGEL® (Corning, CLS354230) in a 1 mL tuberculin syringe. MATRIGEL® was thawed on ice overnight at 4° C. the day before tumor injection. Tumor cells were subcutaneously injected to the right flank of each mouse. Tumor burden was measured based on bioluminescence imaging (BLI) by Xenogen IVIS-200 Spectrum camera (PerkinElmer, Hopkinton, MA, USA). Imaging was performed 10 minutes after intraperitoneal injection of 10 uL/g D-luciferin (15 mg/mL, Gold Biotechnology, St. Louis, MO, USA). 5 days after tumor and macrophage injection, mice were randomized according to tumor burden. Mice were treated with 2×10⁶ CART19 cells or untransduced T cells intravenously by tail vein injection. In the indicated experiments, mice received 10 mg/kg of either IL-1ra neutralizing antibody (clone 10309, Invitrogen, MA5-23802) or IgG control (clone C1.18.4, BioXCell, BE0085) intraperitoneally twice a week for four weeks. Serial BLI was performed to assess tumor burden. Survival endpoint was determined once tumor volume reached to 2000 mm³.

### T Cell Pre-Exposure to Rh IL-18 During CART Production

CART19-28ζ was generated from healthy donor PBMCs as previously described with additional rh IL-1β supplementation. Specifically, T cells were resuspended in TCM to reach 1×10⁶/mL and stimulated with CD3/CD28 beads at a ratio of 1:3 on day 0. T cells were supplemented with or without 100 pg/mL rh IL-1β, 1 ng/mL rh IL-1β, or 10 ng/mL rh IL-1β on the same day. Lentiviral transduction was performed on day 1. T cells were counted from day 3 to day 6 daily to maintain the cell concentration to 1×10⁶/mL by adding fresh TCM supplemented with designated amount of rh IL-1β. CART cells were harvested and washed for intravenous injection in tumor engrafted mice on day 8.

### Mantle Cell Lymphoma Xenograft Stress Mouse Model

NSG mice were injected with 1×10⁶ luciferase⁺ JeKo-1 intravenously. Tumor burden was measured based on bioluminescence imaging (BLI) by Xenogen IVIS-200 Spectrum camera (PerkinElmer, Hopkinton, MA, USA). Imaging was performed 10 minutes after intraperitoneal injection of 10 uL/g D-luciferin (15 mg/mL, Gold Biotechnology, St. Louis, MO, USA). Mice were randomized and treated with 1×10⁶ CART19, CART19 pre-exposed to rh IL-1β, or untransduced T cells intravenously by tail vein injections once tumor burden reached 10⁹ photons/see according to BLI. Tumor burden was analyzed twice a week by BLI. Survival endpoint was determined once 20% of body weight loss was detected in treated mice.

### Immunofluorescence Microscopy

Human M0 macrophages were prepared and resuspended in PBS at concentration of 1×10⁸ cells/mL. Luciferase⁺ JeKo-1 cells were prepared and resuspended in PBS at 2×10⁸ cells/mL. JeKo-1 cells were prepared with macrophages or PBS alone at a 1:1 volume ratio. 100 μL of prepared cells were mixed with the same volume of MATRIGEL®, followed by subcutaneous injection in the right flank of NSG mice. 7 days later, engrafted mice were euthanized, and tumor mass was harvested from subcutaneous tissue. Tumor tissues were fixed with 4% paraformaldehyde at 4° C. for at least 16 hours. Fixed tissues were then placed in cryomolds, and Tissue-Tek O.C.T. Compound (Sakura, 4583) was carefully added to the cryomolds to embed tumor mass, followed by incubation on dry ice. Tissues were then stored at −80° C., cross-sectioned (5 μm) at Mayo Clinic Biomaterials and Histomorphometry Core, and preserved on Micro Slides (Corning, 2948-75X25) at −80° C.

Tumor slides were thawed at room temperature. Excess O.C.T. compound was carefully washed with PBS. Tissues were permeabilized with 0.5% Triton X-100 (Sigma-Aldrich, X100) in PBS for 20 minutes at room temperature. Slides were then carefully washed twice with PBS, followed by blocking with 5% BSA (Sigma-Aldrich, A7906-100G) 0.04% sodium azide (Ricca Chemical, 7144.8) in PBS. Primary antibody, rabbit-anti-human IL-1ra polyclonal antibody (Invitrogen, PA5-13428, 1:20), rabbit-anti-human CD206 monoclonal antibody (clone E2L9N, Cell Signaling Technologies, 91992S, 1:100), was diluted with 5% BSA 0.04% sodium azide in PBS according to manufacturer's suggestions. In the other experiment, human CD206 or iNOS was stained with mouse-anti-human antibody conjugated with AF546 (Santa Cruz Biotechnology, sc-376232 AF546, 1:50) or rabbit-anti-human antibody conjugated with AF647 (Proteintech, CL647-18985, 1:100), respectively. Tissues were incubated with primary antibody overnight at 4° C. in the dark. On the next day, slides were gently washed with PBS. Goat-anti-rabbit IgG (H+L) cross-adsorbed secondary antibody Cyanine5 (Invitrogen, A10523) was diluted with 5% BSA 0.04% sodium azide in PBS at 1:200. Tissues were then incubated with diluted secondary antibodies for 2-3 hours at room temperature in the dark. Slides were washed gently with PBS, followed by a 10-minute incubation with Hoechst 33342 (Thermo Scientific 62249) diluted in PBS (1:2000) at room temperature in the dark. ProLong Glass Antifade Mountant (Invitrogen, P36980) was then applied onto the tissues before placing cover glass (CardinalHealth, M6045-4A). Excess mountant was eliminated by gently pressing cover glass followed by vacuuming. Slides were then stored at room temperature in the dark overnight till confocal imaging. Images were acquired by ZEISS LSM 980 (ZEISS) at 40X.

### Generation of IL-1RI Knockout CART19-28ζ

A single guide RNA (sgRNA) targeting a sequence of gene IL1R1 in exon 5, (5′ AAGTCCTCCGTCTCCTGCAA 3′ (SEQ ID NO:9)) was chosen after screening multiple sgRNAs for knockout efficiency. Non-targeting (CT) sgRNA was used to confirm knockout specificity (5′ GCACTTTGTTTGGCCTACTG 3′ (SEQ ID NO:19)). The sgRNAs were cloned into a CAS9 lentiviral construct under a U6 promoter (lentiCRISPRv2, GenScript, Township, NJ, USA). CART19 production with these sgRNA constructs was conducted as described elsewhere (Sterner et al., J. Vis. Exp., 149: e59629 (2019)). CTsgRNA CART19 and IL-1RI KO CART19 were stimulated with JeKo-1 at a ratio of 1:1. IL-1RI expression on CD4⁺ T cells was measured by flow cytometry to confirm knockout efficiency at the protein level 24 hours after stimulation.

### Statistical Analysis

Flow cytometry data were analyzed by Kaluza (Beckman Coulter, Chaska, MN, USA). GraphPad Prism (La Jolla, CA, USA) was used for statistical analysis. Adjusted P values on dot plots were identified from R package ‘clusterProfiler’ of each cell cluster of interest. P values labeled on violin plots were derived from differential gene expression analysis. Figures were generated by Biorender (Toronto, Ontario, Canada), GraphPad Prism, and Kaluza.

### Example 3: Exemplary Inhibitors of an IL-1RA Polypeptide

This Example provides the amino acid sequences of exemplary anti-IL-1RA antibodies that can be used as an inhibitor of an IL-1RA polypeptide as described herein. The [symbol] of each clone also are provided and delineated.

```
Clone #1 (also referred to as 908.10E12)
VL domain:
(SEQ ID NO: 1)

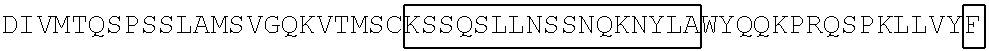

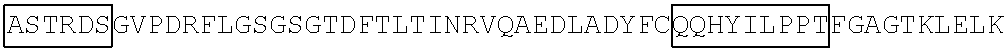


CDR sequences
(SEQ ID NO: 20)
CDR-L1: KSSQSLLNSSNQKNYLA
(SEQ ID NO: 21)
CDR-L2: FASTRDS
(SEQ ID NO: 22)
CDR-L3: QQHYILPPT
VH domain:
(SEQ ID NO: 2)

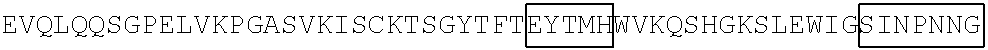

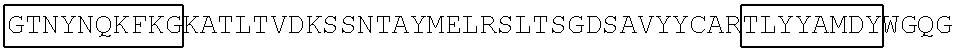


TSVTVSS
CDR sequences
  (SEQ ID NO: 23)
CDR-H1: EYTMH
  (SEQ ID NO: 24)
CDR-H2: SINPNNGGTNYNQKFKG
  (SEQ ID NO: 25)
CDR-H3: TLYYAMDY
Nucleic acid encoding the VL domain:
(SEQ ID NO: 26)
GACATTGTGATGACACAGTCTCCATCCTCCCTGGCTATGTCAGTAGGACAGAAGG
TCACTATGAGCTGCAAGTCCAGTCAGAGCCTTTTAAATAGTAGTAATCAAAAGA
ACTATTTGGCCTGGTACCAGCAGAAACCAAGACAGTCTCCTAAACTTCTGGTATA
CTTTGCATCCACTAGGGATTCTGGGGTCCCTGATCGCTTCTTAGGCAGTGGATCT
GGGACAGATTTCACTCTTACCATCAACCGTGTGCAGGCTGAAGACCTGGCAGATT
ACTTCTGTCAGCAACATTATATTCTTCCTCCCACGTTCGGTGCTGGGACCAAGCT
GGAGCTGAAA
Nucleic acid encoding the VH domain:
(SEQ ID NO: 27)
GAGGTCCAGCTGCAACAGTCTGGACCTGAGCTGGTGAAGCCTGGGGCTTCAGTG
AAGATATCCTGCAAGACTTCTGGATACACATTCACTGAATACACCATGCACTGGG
TGAAGCAGAGCCATGGAAAGAGCCTTGAGTGGATTGGAAGTATTAATCCTAACA
ATGGTGGTACTAACTACAACCAGAAGTTCAAGGGCAAGGCCACATTGACTGTAG
ACAAGTCCTCCAACACAGCCTACATGGAGCTCCGCAGCCTGACATCTGGTGATTC
TGCAGTCTATTACTGTGCAAGAACTCTCTACTATGCTATGGACTACTGGGGTCAA
GGAACCTCAGTCACCGTCTCCTCA
Clone #2 (also referred to as 908.9E11)
VL domain:
(SEQ ID NO: 3)

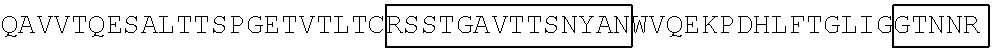

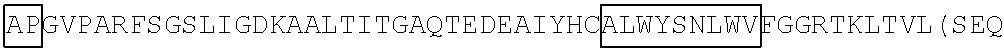


CDR sequences
  (SEQ ID NO: 28)
CDR-L1: RSSTGAVTTSNYAN
(SEQ ID NO: 29)
CDR-L2: GTNNRAP
  (SEQ ID NO: 30)
CDR-L3: ALWYSNLWV
VH domain:
(SEQ ID NO: 4)

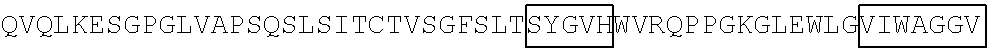

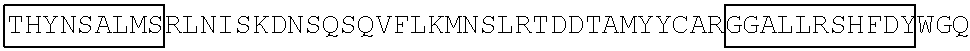


GTTLTVSS
CDR sequences
  (SEQ ID NO: 31)
CDR-H1: SYGVH
  (SEQ ID NO: 32)
CDR-H2: VIWAGGVTHYNSALMS
  (SEQ ID NO: 33)
CDR-H3: GGALLRSHFDY
Nucleic acid encoding the VL domain:
(SEQ ID NO: 34)
CAGGCTGTTGTGACTCAGGAATCTGCACTCACCACATCACCTGGTGAAACAGTC
ACACTCACTTGTCGCTCAAGTACTGGGGCTGTTACAACTAGTAACTATGCCAACT
GGGTCCAAGAAAAACCAGATCATTTATTCACTGGTCTAATAGGTGGTACCAACA
ACCGAGCTCCAGGTGTTCCTGCCAGATTCTCAGGCTCCCTGATTGGAGACAAGGC
TGCCCTCACCATCACAGGGGCACAGACTGAGGATGAGGCAATATATCACTGTGC
TCTATGGTACAGCAACCTTTGGGTGTTCGGTGGAAGAACCAAACTGACTGTCCTA
Nucleic acid encoding the VH domain:
(SEQ ID NO: 35)
CAGGTGCAGCTGAAGGAGTCAGGACCTGGCCTGGTGGCGCCCTCACAGAGCCTG
TCCATCACTTGCACTGTCTCTGGGTTTTCATTAACCAGCTATGGTGTACACTGGGT
TCGCCAGCCTCCAGGAAAGGGTCTGGAGTGGCTGGGAGTAATATGGGCTGGTGG
AGTCACACATTATAATTCGGCTCTCATGTCCAGACTGAACATCAGCAAAGACAA
CTCCCAGAGCCAAGTTTTCTTAAAAATGAACAGTCTACGAACTGATGACACAGC
CATGTACTACTGTGCCAGAGGTGGGGCATTACTTCGGTCTCACTTTGACTACTGG
GGCCAAGGCACCACTCTCACAGTCTCCTCA
Clone #3 (also referred to as 908.9G5)
VL domain:
(SEQ ID NO: 5)

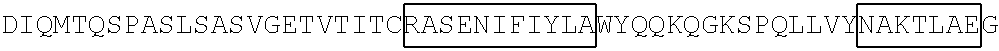

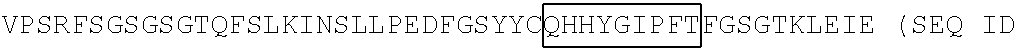


CDR sequences
  (SEQ ID NO: 36)
CDR-L1: RASENIFIYLA
  (SEQ ID NO: 37)
CDR-L2: NAKTLAE
(SEQ ID NO: 38)
CDR-L3: QHHYGIPFT
VH domain:
(SEQ ID NO: 6)

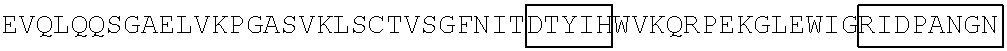

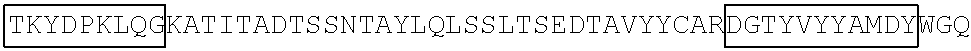


GTSVTVSS
CDR sequences
  (SEQ ID NO: 39)
CDR-H1: DTYIH
  (SEQ ID NO: 40)
CDR-H2: RIDPANGNTKYDPKLQG
  (SEQ ID NO: 41)
CDR-H3: DGTYVYYAMDY
Nucleic acid encoding the VL domain:
(SEQ ID NO: 42)
GACATCCAGATGACTCAGTCTCCAGCCTCCCTATCTGCATCTGTGGGAGAAACTG
TCACCATCACATGTCGAGCAAGTGAGAATATTTTCATTTATTTAGCATGGTATCA
GCAAAAACAGGGAAAATCTCCTCAGCTCCTGGTCTATAATGCAAAGACCTTAGC
AGAAGGTGTGCCATCAAGGTTCAGTGGCAGTGGATCAGGCACACAGTTTTCTCT
GAAGATCAACAGCCTTCTGCCTGAAGATTTTGGGAGTTATTACTGTCAACATCAT
TATGGTATTCCATTCACGTTCGGCTCGGGGACAAAGTTGGAAATAGAA
Nucleic acid encoding the VH domain:
(SEQ ID NO: 43)
GAGGTTCAGCTGCAGCAGTCTGGGGCAGAGCTTGTGAAGCCAGGGGCCTCAGTC
AAGTTGTCCTGCACAGTTTCTGGTTTCAACATTACAGACACCTATATACACTGGG
TGAAGCAGAGGCCTGAAAAGGGCTTGGAGTGGATTGGAAGGATTGATCCTGCGA
ATGGTAATACTAAATATGACCCGAAGTTACAGGGCAAGGCCACTATAACAGCAG
ACACATCCTCCAACACAGCCTACCTGCAGCTCAGCAGCCTGACATCTGAGGACA
CTGCCGTCTATTACTGTGCTAGAGATGGTACCTACGTTTACTATGCTATGGACTA
CTGGGGTCAAGGAACCTCAGTCACCGTCTCCTCA
Clone #4 (also referred to as 908.11A2)
VL domain:
(SEQ ID NO: 7)

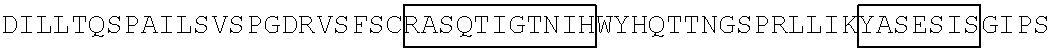

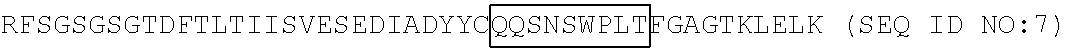


CDR sequences
  (SEQ ID NO: 44)
CDR-L1: RASQTIGTNIH
  (SEQ ID NO: 45)
CDR-L2: YASESIS
(SEQ ID NO: 46)
CDR-L3: QQSNSWPLT
VH domain:
(SEQ ID NO: 8)

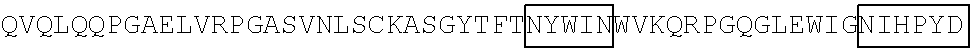

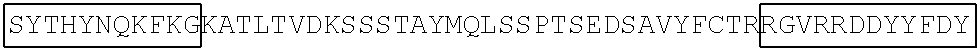


WGQGTTLTVSS
CDR sequences
  (SEQ ID NO: 47)
CDR-H1: NYWIN
  (SEQ ID NO: 48)
CDR-H2: NIHPYDSYTHYNQKFKG
  (SEQ ID NO: 49)
CDR-H3: RGVRRDDYYFDY
Nucleic acid encoding the VL domain:
(SEQ ID NO: 50)
GACATCTTGCTGACTCAGTCTCCAGCCATCCTGTCTGTGAGTCCAGGAGACAGAG
TCAGTTTCTCCTGCAGGGCCAGTCAGACCATTGGCACAAACATACACTGGTATCA
CCAAACAACAAATGGTTCTCCAAGGCTTCTCATTAAGTATGCTTCTGAGTCTATC
TCTGGGATCCCTTCCAGGTTTAGTGGCAGTGGATCAGGGACAGATTTTACTCTTA
CCATCATCAGTGTGGAGTCTGAAGATATTGCAGATTATTACTGTCAACAAAGTAA
TAGCTGGCCGCTCACGTTCGGTGCTGGGACCAAGCTGGAGCTGAAA
Nucleic acid encoding the VH domain:
(SEQ ID NO: 51)
CAGGTCCAACTGCAGCAGCCTGGGGCTGAGCTGGTGAGGCCTGGGGCTTCAGTG
AACCTGTCCTGCAAGGCTTCTGGCTACACCTTCACCAACTACTGGATAAACTGGG
TGAAGCAGAGGCCTGGACAAGGCCTTGAGTGGATCGGAAATATTCATCCTTATG
ATAGTTATACTCACTACAATCAAAAGTTCAAGGGCAAGGCCACATTGACTGTAG
ACAAATCCTCCAGCACGGCCTACATGCAGCTCAGCAGCCCGACATCTGAGGACT
CTGCAGTCTATTTCTGTACAAGGAGGGGGGTACGACGGGACGACTACTACTTTG
ACTACTGGGGCCAAGGCACCACTCTCACAGTCTCCTCA
```

### Example 4: Exemplary Nucleic Acids Encoding an IL-1RI Polypeptide

Nucleic Acid encoding a Human ILIR1 Polypeptide (ENST00000115594)
(SEQ ID NO: 52)
ATGAAAGTGTTACTCAGACTTATTTGTTTCATAGCTCTACTGATTTCTTCTCTGGA
GGCTGATAAATGCAAGGAACGTGAAGAAAAAATAATTTTAGTGTCATCTGCAAA
TGAAATTGATGTTCGTCCCTGTCCTCTTAACCCAAATGAACACAAAGGCACTATA
ACTTGGTATAAAGATGACAGCAAGACACCTGTATCTACAGAACAAGCCTCCAGG
ATTCATCAACACAAAGAGAAACTTTGGTTTGTTCCTGCTAAGGTGGAGGATTCAG
GACATTACTATTGCGTGGTAAGAAATTCATCTTACTGCCTCAGAATTAAAATAAG
TGCAAAATTTGTGGAGAATGAGCCTAACTTATGTTATAATGCACAAGCCATATTT
AAGCAGAAACTACCCGTTGCAGGAGACGGAGGACTTGTGTGCCCTTATATGGAG
TTTTTTAAAAATGAAAATAATGAGTTACCTAAATTACAGTGGTATAAGGATTGCA
AACCTCTACTTCTTGACAATATACACTTTAGTGGAGTCAAAGATAGGCTCATCGT
GATGAATGTGGCTGAAAAGCATAGAGGGAACTATACTTGTCATGCATCCTACAC
ATACTTGGGCAAGCAATATCCTATTACCCGGGTAATAGAATTTATTACTCTAGAG
GAAAACAAACCCACAAGGCCTGTGATTGTGAGCCCAGCTAATGAGACAATGGAA
GTAGACTTGGGATCCCAGATACAATTGATCTGTAATGTCACCGGCCAGTTGAGTG
ACATTGCTTACTGGAAGTGGAATGGGTCAGTAATTGATGAAGATGACCCAGTGC
TAGGGGAAGACTATTACAGTGTGGAAAATCCTGCAAACAAAAGAAGGAGTACCC
TCATCACAGTGCTTAATATATCGGAAATTGAAAGTAGATTTTATAAACATCCATT
TACCTGTTTTGCCAAGAATACACATGGTATAGATGCAGCATATATCCAGTTAATA
TATCCAGTCACTAATTTCCAGAAGCACATGATTGGTATATGTGTCACGTTGACAG
TCATAATTGTGTGTTCTGTTTTCATCTATAAAATCTTCAAGATTGACATTGTGCTT
TGGTACAGGGATTCCTGCTATGATTTTCTCCCAATAAAAGCTTCAGATGGAAAGA
CCTATGACGCATATATACTGTATCCAAAGACTGTTGGGGAAGGGTCTACCTCTGA
CTGTGATATTTTTGTGTTTAAAGTCTTGCCTGAGGTCTTGGAAAAACAGTGTGGA
TATAAGCTGTTCATTTATGGAAGGGATGACTACGTTGGGGAAGACATTGTTGAG
GTCATTAATGAAAACGTAAAGAAAAGCAGAAGACTGATTATCATTTTAGTCAGA
GAAACATCAGGCTTCAGCTGGCTGGGTGGTTCATCTGAAGAGCAAATAGCCATG
TATAATGCTCTTGTTCAGGATGGAATTAAAGTTGTCCTGCTTGAGCTGGAGAAAA
TCCAAGACTATGAGAAAATGCCAGAATCGATTAAATTCATTAAGCAGAAACATG
GGGCTATCCGCTGGTCAGGGGACTTTACACAGGGACCACAGTCTGCAAAGACAA
GGTTCTGGAAGAATGTCAGGTACCACATGCCAGTCCAGCGACGGTCACCTTCAT
CTAAACACCAGTTACTGTCACCAGCCACTAAGGAGAAACTGCAAAGAGAGGCTC
ACGTGCCTCTCGGGTAG
Nucleic Acid encoding a Mouse ILIR1 Polypeptide (ENSMUST00000027241.11)
(SEQ ID NO: 53)
ATGGAGAATATGAAAGTGCTACTGGGGCTCATTTGTCTCATGGTGCCTCTGCTGT
CGCTGGAGATTGACGTATGTACAGAATATCCAAATCAGATCGTTTTGTTTTTATC
TGTAAATGAAATTGATATTCGCAAGTGTCCTCTTACTCCAAATAAAATGCACGGC
GACACCATAATTTGGTACAAGAATGACAGCAAGACCCCCATATCAGCGGACCGG
GACTCCAGGATTCATCAGCAGAATGAACATCTTTGGTTTGTACCTGCCAAGGTGG
AGGACTCAGGATATTACTATTGTATAGTAAGAAACTCAACTTACTGCCTCAAAAC
TAAAGTAACCGTAACTGTGTTAGAGAATGACCCTGGCTTGTGTTACAGCACACA
GGCCACCTTCCCACAGCGGCTCCACATTGCCGGGGATGGAAGTCTTGTGTGCCCT
TATGTGAGTTATTTTAAAGATGAAAATAATGAGTTACCCGAGGTCCAGTGGTATA
AGAACTGTAAACCTCTGCTTCTTGACAACGTGAGCTTCTTCGGAGTAAAAGATAA
ACTGTTGGTGAGGAATGTGGCTGAAGAGCACAGAGGGGACTATATATGCCGTAT
GTCCTATACGTTCCGGGGGAAGCAATATCCGGTCACACGAGTAATACAATTTATC
ACAATAGATGAAAACAAGAGGGACAGACCTGTTATCCTGAGCCCTCGGAATGAG
ACGATCGAAGCTGACCCAGGATCAATGATACAACTGATCTGCAACGTCACGGGC
CAGTTCTCAGACCTTGTCTACTGGAAGTGGAATGGATCAGAAATTGAATGGAAT
GATCCATTTCTAGCTGAAGACTATCAATTTGTGGAACATCCTTCAACCAAAAGAA
AATACACACTCATTACAACACTTAACATTTCAGAAGTTAAAAGCCAGTTTTATCG
CTATCCGTTTATCTGTGTTGTTAAGAACACAAATATTTTTGAGTCGGCGCATGTG
CAGTTAATATACCCAGTCCCTGACTTCAAGAATTACCTCATCGGGGGCTTTATCA
TCCTCACGGCTACAATTGTATGCTGTGTGTGCATCTATAAAGTCTTCAAGGTTGA
CATAGTGCTTTGGTACAGGGACTCCTGCTCTGGTTTTCTTCCTTCAAAAGCTTCAG
ATGGAAAGACATACGATGCCTATATTCTTTATCCCAAGACCCTGGGAGAGGGGT
CCTTCTCAGACTTAGATACTTTTGTTTTTAAACTGTTGCCTGAGGTCTTGGAGGGA
CAGTTTGGATACAAGCTGTTCATTTATGGAAGGGATGACTATGTTGGAGAAGAT
ACCATCGAGGTTACTAATGAAAATGTAAAGAAAAGCAGGAGGCTGATTATCATT
CTAGTGAGAGATATGGGAGGCTTCAGCTGGCTGGGCCAGTCATCTGAAGAGCAA
ATAGCCATATACAATGCTCTCATCCAGGAAGGAATTAAAATCGTCCTGCTTGAGT
TGGAGAAAATCCAAGACTATGAGAAAATGCCAGATTCTATTCAGTTCATTAAGC
AGAAACACGGAGTCATTTGCTGGTCAGGAGACTTTCAAGAAAGACCACAGTCTG
CAAAGACCAGGTTCTGGAAAAACTTAAGATACCAGATGCCAGCCCAACGGAGAT
CACCATTGTCTAAACACCGCTTACTAACCCTGGATCCTGTGCGGGACACTAAGGA
GAAACTGCCGGCAGCAACACACTTACCACTCGGCTAG

### Example 5: Exemplary Cas Sequences

Amino acid sequence of an exemplary Cas9 (N) Polypeptide
(SEQ ID NO: 54)
MDKKYSIGLDIGTNSVGWAVITDEYKVPSKKFKVLGNTDRHSIKKNLIGALLFDSGE
TAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIAQLP
GEKKNGLFGNLIALSLGLTPNFKSNFDLAEDAKLQLSKDTYDDDLDNLLAQIGDQY
ADLFLAAKNLSDAILLSDILRVNTEITKAPLSASMIKRYDEHHQDLTLLKALVRQQLP
EKYKEIFFDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYYVGPLARGNS
RFAWMTRKSEETITPWNFEEVVDKGASAQSFIERMTNFDKNLPNEKVLPKHSLLYE
YFTVYNELTKVKYVTEGMRKPAFLSGEQKKAIVDLLFKTNRKVTVKQLKEDYFKKIE
Nucleic Acid Encoding an Exemplary Cas9 (N) Polypeptide
(SEQ ID NO: 55)
ATGGACAAGAAGTACAGCATCGGCCTGGACATCGGCACCAACTCTGTGGGCTGG
GCCGTGATCACCGACGAGTACAAGGTGCCCAGCAAGAAATTCAAGGTGCTGGGC
AACACCGACCGGCACAGCATCAAGAAGAACCTGATCGGAGCCCTGCTGTTCGAC
AGCGGCGAAACAGCCGAGGCCACCCGGCTGAAGAGAACCGCCAGAAGAAGATA
CACCAGACGGAAGAACCGGATCTGCTATCTGCAAGAGATCTTCAGCAACGAGAT
GGCCAAGGTGGACGACAGCTTCTTCCACAGACTGGAAGAGTCCTTCCTGGTGGA
AGAGGATAAGAAGCACGAGCGGCACCCCATCTTCGGCAACATCGTGGACGAGGT
GGCCTACCACGAGAAGTACCCCACCATCTACCACCTGAGAAAGAAACTGGTGGA
CAGCACCGACAAGGCCGACCTGCGGCTGATCTATCTGGCCCTGGCCCACATGAT
CAAGTTCCGGGGCCACTTCCTGATCGAGGGCGACCTGAACCCCGACAACAGCGA
CGTGGACAAGCTGTTCATCCAGCTGGTGCAGACCTACAACCAGCTGTTCGAGGA
AAACCCCATCAACGCCAGCGGCGTGGACGCCAAGGCCATCCTGTCTGCCAGACT
GAGCAAGAGCAGACGGCTGGAAAATCTGATCGCCCAGCTGCCCGGCGAGAAGA
AGAATGGCCTGTTCGGAAACCTGATTGCCCTGAGCCTGGGCCTGACCCCCAACTT
CAAGAGCAACTTCGACCTGGCCGAGGATGCCAAACTGCAGCTGAGCAAGGACAC
CTACGACGACGACCTGGACAACCTGCTGGCCCAGATCGGCGACCAGTACGCCGA
CCTGTTTCTGGCCGCCAAGAACCTGTCCGACGCCATCCTGCTGAGCGACATCCTG
AGAGTGAACACCGAGATCACCAAGGCCCCCCTGAGCGCCTCTATGATCAAGAGA
TACGACGAGCACCACCAGGACCTGACCCTGCTGAAAGCTCTCGTGCGGCAGCAG
CTGCCTGAGAAGTACAAAGAGATTTTCTTCGACCAGAGCAAGAACGGCTACGCC
GGCTACATTGACGGCGGAGCCAGCCAGGAAGAGTTCTACAAGTTCATCAAGCCC
ATCCTGGAAAAGATGGACGGCACCGAGGAACTGCTCGTGAAGCTGAACAGAGA
GGACCTGCTGCGGAAGCAGCGGACCTTCGACAACGGCAGCATCCCCCACCAGAT
CCACCTGGGAGAGCTGCACGCCATTCTGCGGCGGCAGGAAGATTTTTACCCATTC
CTGAAGGACAACCGGGAAAAGATCGAGAAGATCCTGACCTTCCGCATCCCCTAC
TACGTGGGCCCTCTGGCCAGGGGAAACAGCAGATTCGCCTGGATGACCAGAAAG
AGCGAGGAAACCATCACCCCCTGGAACTTCGAGGAAGTGGTGGACAAGGGCGCT
TCCGCCCAGAGCTTCATCGAGCGGATGACCAACTTCGATAAGAACCTGCCCAAC
GAGAAGGTGCTGCCCAAGCACAGCCTGCTGTACGAGTACTTCACCGTGTATAAC
GAGCTGACCAAAGTGAAATACGTGACCGAGGGAATGAGAAAGCCCGCCTTCCTG
AGCGGCGAGCAGAAAAAGGCCATCGTGGACCTGCTGTTCAAGACCAACCGGAA
AGTGACCGTGAAGCAGCTGAAAGAGGACTACTTCAAGAAAATCGAG
Amino acid sequence of an exemplary Cas9 (C) Polypeptide
(SEQ ID NO: 56)
KPAFLSGEQKKAIVDLLFKTNRKVTVKQLKEDYFKKIECFDSVEISGVEDRFNASLGT
YHDLLKIIKDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDSLTFKEDI
QKAQVSGQGDSLHEHIANLAGSPAIKKGILQTVKVVDELVKVMGRHKPENIVIEMA
RENQTTQKGQKNSRERMKRIEEGIKELGSQILKEHPVENTQLQNEKLYLYYLQNGRD
MYVDQELDINRLSDYDVDHIVPQSFLKDDSIDNKVLTRSDKNRGKSDNVPSEEVVK
KMKNYWRQLLNAKLITQRKFDNLTKAERGGLSELDKAGFIKRQLVETRQITKHVAQ
ILDSRMNTKYDENDKLIREVKVITLKSKLVSDFRKDFQFYKVREINNYHHAHDAYLN
AVVGTALIKKYPKLESEFVYGDYKVYDVRKMIAKSEQEIGKATAKYFFYSNIMNFF
KTEITLANGEIRKRPLIETNGETGEIVWDKGRDFATVRKVLSMPQVNIVKKTEVQTG
GFSKESILPKRNSDKLIARKKDWDPKKYGGFDSPTVAYSVLVVAKVEKGKSKKLKS
VKELLGITIMERSSFEKNPIDFLEAKGYKEVKKDLIIKLPKYSLFELENGRKRMLASA
GELQKGNELALPSKYVNFLYLASHYEKLKGSPEDNEQKQLFVEQHKHYLDEIIEQISE
FSKRVILADANLDKVLSAYNKHRDKPIREQAENIIHLFTLTNLGAPAAFKYFDTTIDR
KRYTSTKEVLDATLIHQSITGLYETRIDLSQLGGD
Nucleic Acid Encoding an Exemplary Cas9 (C) Polypeptide
(SEQ ID NO: 57)
AAGCCCGCCTTCCTGAGCGGCGAGCAGAAAAAGGCCATCGTGGACCTGCTGTTC
AAGACCAACCGGAAAGTGACCGTGAAGCAGCTGAAAGAGGACTACTTCAAGAA
AATCGAGTGCTTCGACTCCGTGGAAATCTCCGGCGTGGAAGATCGGTTCAACGC
CTCCCTGGGCACATACCACGATCTGCTGAAAATTATCAAGGACAAGGACTTCCT
GGACAATGAGGAAAACGAGGACATTCTGGAAGATATCGTGCTGACCCTGACACT
GTTTGAGGACAGAGAGATGATCGAGGAACGGCTGAAAACCTATGCCCACCTGTT
CGACGACAAAGTGATGAAGCAGCTGAAGCGGCGGAGATACACCGGCTGGGGCA
GGCTGAGCCGGAAGCTGATCAACGGCATCCGGGACAAGCAGTCCGGCAAGACA
ATCCTGGATTTCCTGAAGTCCGACGGCTTCGCCAACAGAAACTTCATGCAGCTGA
TCCACGACGACAGCCTGACCTTTAAAGAGGACATCCAGAAAGCCCAGGTGTCCG
GCCAGGGCGATAGCCTGCACGAGCACATTGCCAATCTGGCCGGCAGCCCCGCCA
TTAAGAAGGGCATCCTGCAGACAGTGAAGGTGGTGGACGAGCTCGTGAAAGTGA
TGGGCCGGCACAAGCCCGAGAACATCGTGATCGAAATGGCCAGAGAGAACCAG
ACCACCCAGAAGGGACAGAAGAACAGCCGCGAGAGAATGAAGCGGATCGAAGA
GGGCATCAAAGAGCTGGGCAGCCAGATCCTGAAAGAACACCCCGTGGAAAACA
CCCAGCTGCAGAACGAGAAGCTGTACCTGTACTACCTGCAGAATGGGCGGGATA
TGTACGTGGACCAGGAACTGGACATCAACCGGCTGTCCGACTACGATGTGGACC
ATATCGTGCCTCAGAGCTTTCTGAAGGACGACTCCATCGACAACAAGGTGCTGA
CCAGAAGCGACAAGAACCGGGGCAAGAGCGACAACGTGCCCTCCGAAGAGGTC
GTGAAGAAGATGAAGAACTACTGGCGGCAGCTGCTGAACGCCAAGCTGATTACC
CAGAGAAAGTTCGACAATCTGACCAAGGCCGAGAGAGGCGGCCTGAGCGAACT
GGATAAGGCCGGCTTCATCAAGAGACAGCTGGTGGAAACCCGGCAGATCACAA
AGCACGTGGCACAGATCCTGGACTCCCGGATGAACACTAAGTACGACGAGAATG
ACAAGCTGATCCGGGAAGTGAAAGTGATCACCCTGAAGTCCAAGCTGGTGTCCG
ATTTCCGGAAGGATTTCCAGTTTTACAAAGTGCGCGAGATCAACAACTACCACCA
CGCCCACGACGCCTACCTGAACGCCGTCGTGGGAACCGCCCTGATCAAAAAGTA
CCCTAAGCTGGAAAGCGAGTTCGTGTACGGCGACTACAAGGTGTACGACGTGCG
GAAGATGATCGCCAAGAGCGAGCAGGAAATCGGCAAGGCTACCGCCAAGTACT
TCTTCTACAGCAACATCATGAACTTTTTCAAGACCGAGATTACCCTGGCCAACGG
CGAGATCCGGAAGCGGCCTCTGATCGAGACAAACGGCGAAACCGGGGAGATCG
TGTGGGATAAGGGCCGGGATTTTGCCACCGTGCGGAAAGTGCTGAGCATGCCCC
AAGTGAATATCGTGAAAAAGACCGAGGTGCAGACAGGCGGCTTCAGCAAAGAG
TCTATCCTGCCCAAGAGGAACAGCGATAAGCTGATCGCCAGAAAGAAGGACTGG
GACCCTAAGAAGTACGGCGGCTTCGACAGCCCCACCGTGGCCTATTCTGTGCTG
GTGGTGGCCAAAGTGGAAAAGGGCAAGTCCAAGAAACTGAAGAGTGTGAAAGA
GCTGCTGGGGATCACCATCATGGAAAGAAGCAGCTTCGAGAAGAATCCCATCGA
CTTTCTGGAAGCCAAGGGCTACAAAGAAGTGAAAAAGGACCTGATCATCAAGCT
GCCTAAGTACTCCCTGTTCGAGCTGGAAAACGGCCGGAAGAGAATGCTGGCCTC
TGCCGGCGAACTGCAGAAGGGAAACGAACTGGCCCTGCCCTCCAAATATGTGAA
CTTCCTGTACCTGGCCAGCCACTATGAGAAGCTGAAGGGCTCCCCCGAGGATAA
TGAGCAGAAACAGCTGTTTGTGGAACAGCACAAGCACTACCTGGACGAGATCAT
CGAGCAGATCAGCGAGTTCTCCAAGAGAGTGATCCTGGCCGACGCTAATCTGGA
CAAAGTGCTGTCCGCCTACAACAAGCACCGGGATAAGCCCATCAGAGAGCAGGC
CGAGAATATCATCCACCTGTTTACCCTGACCAATCTGGGAGCCCCTGCCGCCTTC
AAGTACTTTGACACCACCATCGACCGGAAGAGGTACACCAGCACCAAAGAGGTG
CTGGACGCCACCCTGATCCACCAGAGCATCACCGGCCTGTACGAGACACGGATC
GACCTGTCTCAGCTGGGAGGCGAC

### Example 6: Treating Cancer

A human identified as having cancer is administered one or more inhibitors of IL-1RA polypeptide and is administered CAR T cells. The administered inhibitor(s) can increase an IL-1 signaling pathway (e.g., an IL-1β signaling pathway) to reduce immunosuppression of the administered cells CAR T cells and the administered CAR T cells can target (e.g., target and destroy) cancer cells (e.g., cancer cells expressing a tumor antigen targeted by the CAR T cells) within the human to reduce the number of cancer cells present within the human.

### Example 7: Treating Cancer

A human having cancer is administered CAR T cells having a reduced level of an IL-1R1 polypeptide.

The administered CAR T cells having a reduced level of an IL-1R1 polypeptide can target (e.g., target and destroy) cancer cells (e.g., cancer cells expressing a tumor antigen targeted by the CAR T cells) within a mammal.

### Example 8: Treating Cancer

T cells are obtained from a mammal having cancer and are engineered to be CAR T cells having a reduced level of an IL-1R1 polypeptide.

The CAR T cells having a reduced level of an IL-1R1 polypeptide are administered back to the human.

The administered CAR T cells having a reduced level of an IL-1R1 polypeptide can target (e.g., target and destroy) cancer cells (e.g., cancer cells expressing a tumor antigen targeted by the CAR T cells) within the mammal.

### Example 9: Treating Cancer

A human having cancer is administered one or more inhibitors of IL-1RA polypeptide and is administered CAR T cells having a reduced level of an IL-1R1 polypeptide.

The administered one or more inhibitors of IL-1RA polypeptide can reduce immunosuppression of the administered CAR T cells and the administered CAR T cells having a reduced level of an IL-1R1 polypeptide can target (e.g., target and destroy) cancer cells (e.g., cancer cells expressing a tumor antigen targeted by the CAR T cells) within the mammal.

### Example 10: Treating Cancer

T cells are obtained from a mammal having cancer and are engineered to be CAR T cells having a reduced level of an IL-1R1 polypeptide.

The CAR T cells having a reduced level of an IL-1R1 polypeptide are administered back to the human together with one or more inhibitors of IL-1RA polypeptide.

The administered inhibitor(s) of an IL-1RA polypeptide can reduce immunosuppression of the administered CAR T cells and the administered CAR T cells having a reduced level of an IL-1R1 polypeptide can target (e.g., target and destroy) cancer cells (e.g., cancer cells expressing a tumor antigen targeted by the CAR T cells) within a mammal.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

The invention claimed is:

1. A method for treating a mammal having cancer, wherein said method comprises administering, to said mammal, (a) an inhibitor of an interleukin-1 receptor antagonist (IL-1RA) polypeptide and (b) an adoptive cell therapy, wherein a number of cancer cells within the mammal is reduced.

2. The method of claim 1, wherein said mammal is a human.

3. The method of claim 1, wherein said cancer is selected from the group consisting of a mantle cell lymphoma (MCL), a diffuse large B cell lymphoma (DLBCL), a Hodgkin's lymphoma, a non-Hodgkin lymphoma, an acute lymphoblastic leukemia (ALL), a chronic lymphocytic leukemia (CLL), an acute myeloid leukemia (AML), a germ cell tumor, a hepatocellular carcinoma, a bowel cancer, a lung cancer, a breast cancer, an ovarian cancer, a melanoma, a brain cancer, and a multiple myeloma.

4-5. (canceled)

6. The method of claim 1, wherein said inhibitor of said IL-1RA polypeptide is an anti-IL-1RA antibody.

7. The method of claim 1, wherein said adoptive cell therapy is a chimeric antigen receptor (CAR) T cell therapy.

8. The method of claim 7, wherein said CAR targets a tumor-associated antigen.

9-12. (canceled)

13. A T cell having a reduced likelihood of causing a CAR T cell-associated toxicity, wherein said T cell comprises (a) a reduced level of an interleukin 1 receptor, type I (IL-1R1) polypeptide, and (b) nucleic acid encoding a chimeric antigen receptor (CAR), and wherein said T cell expresses said CAR.

14. The T cell of claim 13, wherein said T cell comprises a disruption in at least one endogenous allele encoding said IL-1R1 polypeptide.

15. The T cell of claim 13, wherein said T cell comprises a disruption in both endogenous alleles encoding said IL-1R1 polypeptide.

16. (canceled)

17. The T cell of claim 13, wherein said CAR targets a tumor-associated antigen.

18. The T cell of claim 13, wherein said T cell is obtained from a human.

19. The T cell of claim 13, wherein said CAR T cell toxicity is selected from the group consisting of a cytokine release syndrome (CRS) and an immune effector cell-associated neurotoxicity syndrome (ICANS).

20. A method for treating a mammal having cancer, wherein said method comprises administering, to said mammal, a composition comprising a T cell having a reduced likelihood of causing a CAR T cell-associated toxicity, wherein said T cell comprises (a) a reduced level of an interleukin 1 receptor, type I (IL-1R1) polypeptide, and (b) nucleic acid encoding a chimeric antigen receptor (CAR), and wherein said T cell expresses said CAR.

21. The method of claim 20, wherein said mammal is a human.

22. The method of claim 20, wherein said cancer is selected from the group consisting of a MCL, a DLBCL, a Hodgkin's lymphoma, a non-Hodgkin lymphoma, an ALL, a CLL, an AML, a germ cell tumor, a hepatocellular carcinoma, a bowel cancer, a lung cancer, a breast cancer, an ovarian cancer, a melanoma, a brain cancer, and a multiple myeloma.

23. The method of claim 20, wherein said CAR targets a tumor-associated antigen.

24. A method for providing a mammal with CAR T cells having a reduced likelihood of inducing a CAR T cell-associated toxicity, wherein said method comprises administering, to said mammal, a composition comprising a T cell, wherein said T cell comprises (a) a reduced level of an interleukin 1 receptor, type I (IL-1R1) polypeptide, and (b) nucleic acid encoding a chimeric antigen receptor (CAR), and wherein said T cell expresses said CAR, and wherein said CAR T cells do not induce said CAR T cell-associated toxicity as rapidly as comparable CAR T cells not having said reduced level of said IL-1R1 polypeptide administered to a comparable mammal.

25. The method of claim 24, wherein said mammal is a human.

26. The method of claim 24, wherein said cancer is selected from the group consisting of a MCL, a DLBCL, a Hodgkin's lymphoma, a non-Hodgkin lymphoma, an ALL, a CLL, an AML, a germ cell tumor, a hepatocellular carcinoma, a bowel cancer, a lung cancer, a breast cancer, an ovarian cancer, a melanoma, a brain cancer, and a multiple myeloma.

27. The method of claim 24, wherein said CAR targets a tumor-associated antigen.

28. The method of claim 24, wherein said CAR T cell toxicity is selected from the group consisting of a CRS and an ICANS.

29-31. (canceled)

32. A method for providing a mammal with CAR T cells having a reduced susceptibility to T cell immunosuppression, wherein said method comprises:
(a) administering a composition comprising CAR T cells to said mammal, and
(b) administering an inhibitor of an IL-1RA polypeptide to said mammal,
wherein said CAR T cells do not exhibit T cell immunosuppression within said mammal as rapidly as comparable CAR T cells administered to a comparable mammal not administered said inhibitor of said IL-1RA polypeptide.

33. The method of claim 32, wherein said mammal is a human.

34. The method of claim 32, wherein said CAR T cells target a tumor-associated antigen.

35-36. (canceled)

37. The method of claim 32, wherein said inhibitor of said IL-1RA polypeptide is an anti-IL-1RA antibody.

38-40. (canceled)
